(19) United States
(12) Patent Application Publication
(10) Pub. No.: US 2017/0192004 A1
(43) Pub. Date: Jul. 6, 2017

(54) Methods and Arrays for Use in the Same

(71) Applicant: IMMUNOVIA AB, Lund (SE)

(72) Inventors: Carl Arne Krister Borrebaeck, Lund (SE); Christer Lars Bertil Wingren, Sodra, Sandby (SE)

(21) Appl. No.: 15/314,646

(22) PCT Filed: Jun. 9, 2015
(86) PCT No.: PCT/GB2015/051678

§ 371 Date: Nov. 29, 2016

(30) Foreign Application Priority Data
Jun. 9, 2014 (GB) 1410226.3

(51) Int. Cl. G01N 33/574; C40B 40/00; C40B 30/00

(52) U.S. Cl. CPC G01N 33/57415; C40B 30/00; C40B 40/00; G01N 33/53

(57) ABSTRACT

The present invention provides a method for diagnosing breast cancer comprising or consisting of the steps of (a) providing a sample to be tested; and (b) determining a biomarker signature of the test sample by measuring the presence and/or amount in the test sample of one or more biomarker selected from the group defined in Table A(i) and/or Table A(ii); wherein the presence and/or amount in the test sample of the one or more biomarker selected from the group defined in Table A(i) and/or Table A(ii) is indicative of the presence of breast cancer cells in the individual, corresponding uses, methods of treating breast cancer, together with arrays and kits for use in the same.

### FIELD OF THE INVENTION

The present invention provides methods for determining a breast cancer-associated disease state, as well as arrays and kits for use in such methods.

### BACKGROUND OF THE INVENTION

Breast cancer (BC) is the second most common newly diagnosed cancer and second leading cause of cancer death among women in the US. Serological profiling of early BC is an attractive approach for deciphering disease-associated serum biomarkers, which could pave the way for early and improved detection and diagnosis, as well as an enhanced understanding of the underlying disease biology and processes involved in early BC (1-4). In fact, serum protein profiling efforts have indicated that the serum profiles might be altered already up to three years before clinical BC detection and diagnosis (1, 2).

The immune response is involved early on in the development of cancer, including BC (4), and the interplay between the immune system and cancer in general has been the subject of major attention and controversies for a long time (5). Based on extensive work, the immune surveillance theory originally proposed more than 50 years ago (6) has been refined and extended into the concept of ‘cancer immunoediting’ (7, 8). During cancer immunoediting, the immune system of the host shapes tumor fate, in three consecutive phases—Elimination, Equilibrium, and Escape—through the activation of both innate and adaptive immune mechanisms (9-11). In the first phase, the transformed cells are destroyed by a competent immune system long before the tumor becomes clinically apparent (5, 12, 13). Sporadic tumor cells that manage to survive then enter the equilibrium phase, in which a balance is established between the immune system and the tumor, shaping each other reciprocally. Immunologically sculptured tumors then enter the escape phase, in which their outgrowth is no longer blocked by immunity, and they become clinically apparent and establish an immunosuppressive tumor microenvironment (5, 12-15). Notably, escape from immune control is now established and recognized to be one of the hallmarks of cancer (16). The view of these three phases on a detailed molecular level has begun to emerge, predominantly described in the tumor microenvironment in mice or mice and humans, with the balance of key anti-tumor (e.g. IL-1, IL-12, IFN-γ) and tumor promoting (immunosuppressive) cytokines (e.g. IL-10, IL-23, TGF-β, and VEGF) playing a central role (5, 7, 9-11). Still, further studies of early cancer in particular in humans will be required before all participating molecules and their exact interplay will be unraveled. In the long run, this could provide novel opportunities for deciphering diagnostic, immune predictive and diagnostic biomarkers in early BC and cancer in general.

In this context, we have previously developed a recombinant antibody microarray technology platform for multiplexed protein expression profiling of crude proteomes, such as serum (17-19). Focusing on BC, we have successfully applied the technology for decoding multiplexed serum biomarker signatures associated with metastasis of BC (20) and predicting the risk of developing distant metastasis (21). Notably, the array set-up was designed to harvest the immune system as a specific and sensitive sensor for disease, by targeting predominantly immunoregulatory and cancer-associated analytes (17, 22, 23). Nevertheless, early intervention is crucial for the effective treatment of BC. There remains a need to identify diagnostic biomarkers for BC, in particular biomarkers capable of diagnosing BC prior to the manifestation of clinical symptoms.

### DISCLOSURE OF THE INVENTION

The present inventors have now, for the first time, applied recombinant antibody array technology to perform immunoprofiling of early human BC by targeting human serum samples collected up to two years before clinical diagnosis. The results show that several disease-progression associated biomarkers could be deciphered in early human BC. Hence, the observed serological profiles shed light on the biological processes involved in early BC, such as cancer immunoediting, and provide novel opportunities for early BC classification and diagnosis. Early detection of BC is a crucial development since, in many cases, the sooner cancer is diagnosed and treated, the better an individual's prognosis will be.

Accordingly, a first aspect of the invention provides a method for diagnosing breast cancer comprising or consisting of the steps of:
a) providing a sample to be tested; and
b) determining a biomarker signature of the test sample by measuring the presence and/or amount in the test sample of one or more biomarker selected from the group defined in Table A(i) and/or Table A(ii);
wherein the presence and/or amount in the test sample of the one or more biomarker selected from the group defined in Table A(i) and/or Table A(ii) is indicative of the presence of breast cancer cells in the individual.

By “biomarker” we mean a naturally-occurring biological molecule, or component or fragment thereof, the measurement of which can provide information useful in the prognosis of breast cancer. For example, the biomarker may be a naturally-occurring protein or carbohydrate moiety, or an antigenic component or fragment thereof.

Preferably the sample to be tested is provided from a mammal. The mammal may be any domestic or farm animal. Preferably, the mammal is a rat, mouse, guinea pig, cat, dog, horse or a primate. Most preferably, the mammal is human. Preferably the sample is a cell or tissue sample (or derivative thereof) comprising or consisting of breast cancer cells or equally preferred, protein or nucleic acid derived from a cell or tissue sample comprising or consisting of breast cancer cells. Preferably test and control samples are derived from the same species.

In an alternative or additional embodiment, by “the amount in the test sample of the one or more biomarker selected from the group defined in Table A(i) and/or Table A(ii) is indicative of the presence of breast cancer cells in the individual” we include that the amount of the one or more biomarker demonstrates the same regulatory trend as shown in the tables and figures of the present application and/or one or more positive control sample (i.e., up-regulation or down-regulation, as appropriate).

In an alternative or additional embodiment, by “the amount in the test sample of the one or more biomarker selected from the group defined in Table A(i) and/or Table A(ii) is indicative of the presence of breast cancer cells in the individual” we include that the amount of the one or more biomarker corresponds to the amount shown in the tables and figures of the present application and/or one or more positive control sample (i.e., corresponding levels of up-regulation or down-regulation, as appropriate).

By “corresponds to the amount” we mean the presence and or amount is identical to that shown in the tables and figures of the present application and/or one or more positive control sample or is at least 60% of it, for example, at least 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an alternative or additional embodiment, the breast cancer is early breast cancer.

By “early breast cancer” we include breast cancers that have not been diagnosed by conventional clinical methods.

Alternatively and/or additionally, by “early breast cancer” we include breast cancers that are of insufficient size and/or developmental stage to be diagnosed by conventional clinical methods.

The contemporary best practice for clinical breast cancer diagnosis will be well known to the person of skill in the art, however, for a detailed review see “Best practice diagnostic guidelines for patients presenting with breast symptoms guideline; review date: November 2012, Cancer Reform Strategy Breast Cancer Working Group, Editors Alexis M Willett, Michael J Michell, Martin J R Lee” which is incorporated by reference herein (obtainable here: http://www.associationofbreastsurgery.org.uk/media/4585/best_practice_diagnostic_guidelines_for_patients_presenting_with_breast_symptoms.pdf). In one embodiment, by “conventional clinical diagnoses” (and the like [e.g., “diagnosed by conventional clinical methods”]) we include mammogram, ultrasound, histopathology and/or physical examination (e.g., of the breasts and, possibly, local lymph nodes). In one embodiment by “conventional clinical diagnoses” (and the like) we include the breast cancer diagnosis procedures set out in “Best practice diagnostic guidelines for patients presenting with breast symptoms guideline; review date: November 2012, Cancer Reform Strategy Breast Cancer Working Group, Editors Alexis M Willett, Michael J Michell, Martin J R Lee”.

In an alternative or additional embodiment by “conventional clinical diagnoses” (and the like) we exclude the use of molecular biomarkers present in bodily fluids (such as blood, serum, interstitial fluid, lymph, urine, mucus, saliva, sputum, sweat).

In an alternative or additional embodiment by “early breast cancer” we include breast cancers comprising tumours of 20 mm or less in all dimensions (i.e., in this embodiment individuals with early breast cancer do not comprise breast cancer tumours of greater than 20 mm in any dimension), for example, equal to or less than 19 mm, 18 mm, 17 mm, 16 mm, 15 mm, 14 mm, 13 mm, 12 mm, 11 mm, 10 mm, 9 mm, 8 mm, 7 mm, 6 mm, 5 mm, 4 mm, 3 mm, 2 mm, 1 mm or equal to or 0.1 mm in all dimensions. In an alternative or additional embodiment, the breast cancer tumours of 20 mm or less in all dimensions are at least 2 mm in one dimension. In an alternative or additional embodiment, the breast cancer tumours of 20 mm or less in all dimensions are at least 2 mm all dimensions.

In an alternative or additional embodiment step (b) comprises or consists of measuring the presence and/or amount of 1 or more biomarker listed in Table A, for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113 or 114 of the biomarkers listed in Table A.

In an alternative or additional embodiment step (b) comprises or consists of measuring the presence and/or amount of 1 or more biomarker listed in Table A(i), for example at least 2 or 3 of the biomarkers listed in Table A(i).

In an alternative or additional embodiment step (b) comprises or consists of measuring the presence and/or amount of 1 or more biomarker listed in Table A(ii), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65 of the biomarkers listed in Table A(ii).

In an alternative or additional embodiment step (b) comprises or consists of measuring the presence and/or amount of 1 or more biomarker listed in Table A(iii), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or 46 of the biomarkers listed in Table A(iii).

In an alternative or additional embodiment step (b) comprises or consists of measuring the presence and/or amount of 1 or more biomarker listed in Table B(i), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68 of the biomarkers listed in Table B(i).

In an alternative or additional embodiment step (b) comprises or consists of measuring the presence and/or amount of 1 or more biomarker listed in Table B(ii), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 of the biomarkers listed in Table B(ii). Hence, the method may be indicative of whether or not the test sample is characteristic of a sample taken from an individual with breast cancer 0-52 weeks prior to diagnosis by conventional clinical methods.

By “characteristic of a breast cancer diagnosed by conventional clinical methods within X weeks” (wherein “X” means any number or number range) we include that the test sample breast cancer has morphological, histological and/or biochemical features that correspond to that of one or more reference or control breast cancer. In particular, we include that the biomarker profile of the breast cancer in the test sample corresponds to the biomarker profile of one or more reference or control breast cancer, in particular, a biomarker profile of the present invention.

In an alternative or additional embodiment step (b) comprises or consists of measuring the presence and/or amount of 1 or more biomarker listed in Table B(iii), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 or 43 of the biomarkers listed in Table B(iii). Hence, the method may be indicative of whether or not the test sample is characteristic of a sample taken from an individual with breast cancer 52-104 weeks prior to diagnosis by conventional clinical methods.

In an alternative or additional embodiment step (b) comprises or consists of measuring the presence and/or amount of 1 or more biomarker listed in Table B(iv), for example at least 2, 3, 4, 5, 6, 7, 8, 9, 10, 11, 12, 13, 14, 15, 16, 17, 18, 19, 20 or 21 of the biomarkers listed in Table B(iv). Hence, the method may be indicative of whether or not the test sample is characteristic of a sample taken from an individual with breast cancer 0-26 weeks prior to diagnosis by conventional clinical methods.

In an alternative or additional embodiment step (b) comprises or consists of measuring the presence and/or amount of 1 or more biomarker listed in Table B(v), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77 or 78 of the biomarkers listed in Table B(v). Hence, the method may be indicative of whether or not the test sample is characteristic of a sample taken from an individual with breast cancer 26-52 weeks prior to diagnosis by conventional clinical methods.

In an alternative or additional embodiment step (b) comprises or consists of measuring the presence and/or amount of 1 or more biomarker listed in Table B(vi), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 of the biomarkers listed in Table B(vi). Hence, the method may be indicative of whether or not the test sample is characteristic of a sample taken from an individual with breast cancer 52-78 weeks prior to diagnosis by conventional clinical methods.

In an alternative or additional embodiment step (b) comprises or consists of measuring the presence and/or amount of 1 or more biomarker listed in Table B(vii), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 of the biomarkers listed in Table B(vii). Hence, the method may be indicative of whether or not the test sample is characteristic of a sample taken from an individual with breast cancer 78-104 weeks prior to diagnosis by conventional clinical methods.

In an alternative or additional embodiment step (b) comprises or consists of measuring the presence and/or amount of 1 or more biomarker listed in Table B(viii). Hence, the method may be indicative of whether or not the test sample is characteristic of a sample taken from an individual with breast cancer 0-35 weeks prior to diagnosis by conventional clinical methods of breast cancer consisting of tumours of less than or equal to 20 mm in any dimension.

In an alternative or additional embodiment step (b) comprises or consists of measuring the presence and/or amount of 1 or more biomarker listed in Table B(ix), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 of the biomarkers listed in Table B(ix). Hence, the method may be indicative of whether or not the test sample is characteristic of a sample taken from an individual with breast cancer 70-104 weeks prior to diagnosis by conventional clinical methods of breast cancer consisting of tumours of less than or equal to 20 mm in any dimension.

In an alternative or additional embodiment step (b) comprises or consists of measuring the presence and/or amount of 1 or more biomarker listed in Table B(x), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62 or 63 of the biomarkers listed in Table B(x). Hence, the method may be indicative of whether or not the test sample is characteristic of a sample taken from an individual with breast cancer 0-26 or 26-52 weeks prior to diagnosis by conventional clinical methods.

In an alternative or additional embodiment step (b) comprises or consists of measuring the presence and/or amount of 1 or more biomarker listed in Table B(xi), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81 or 82 of the biomarkers listed in Table B(xi). Hence, the method may be indicative of whether or not the test sample is characteristic of a sample taken from an individual with breast cancer 26-52 or 52-78 weeks prior to diagnosis by conventional clinical methods.

In an alternative or additional embodiment step (b) comprises or consists of measuring the presence and/or amount of 1 or more biomarker listed in Table B(xii), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 or 47 of the biomarkers listed in Table B(xii). Hence, the method may be indicative of whether or not the test sample is characteristic of a sample taken from an individual with breast cancer 52-78 or 78-104 weeks prior to diagnosis by conventional clinical methods.

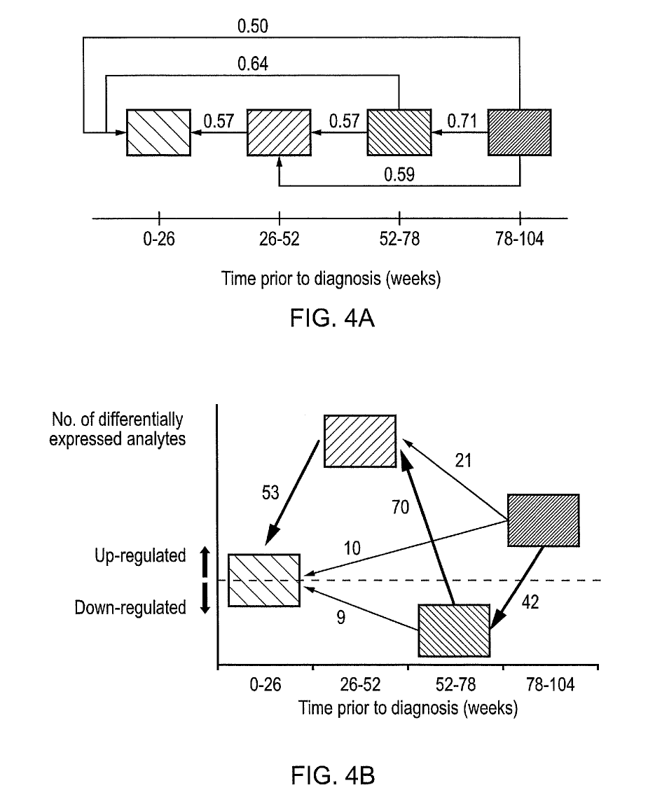

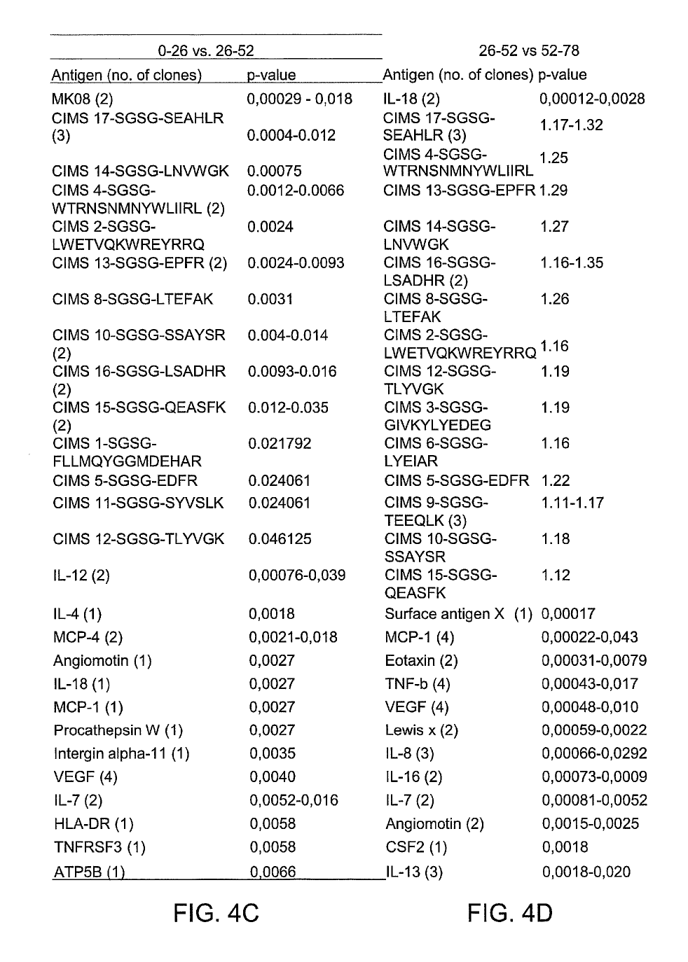

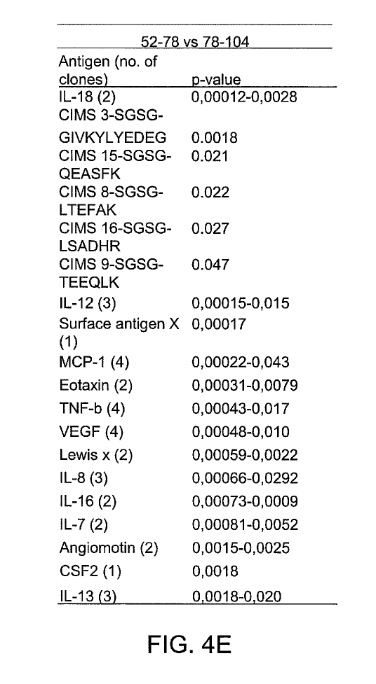

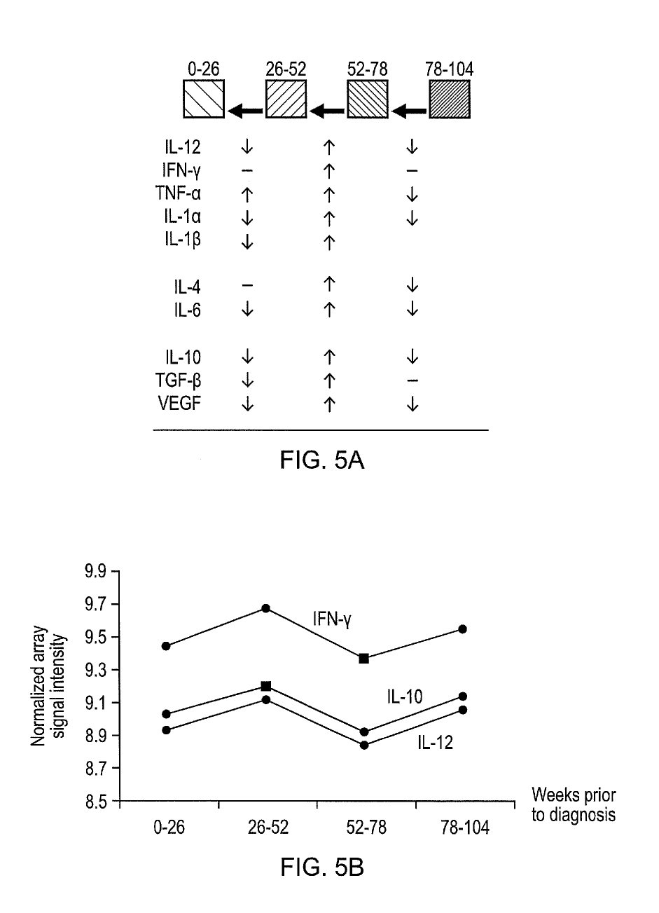

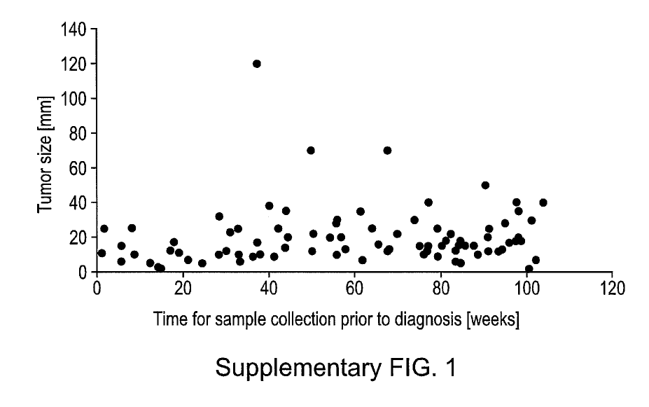

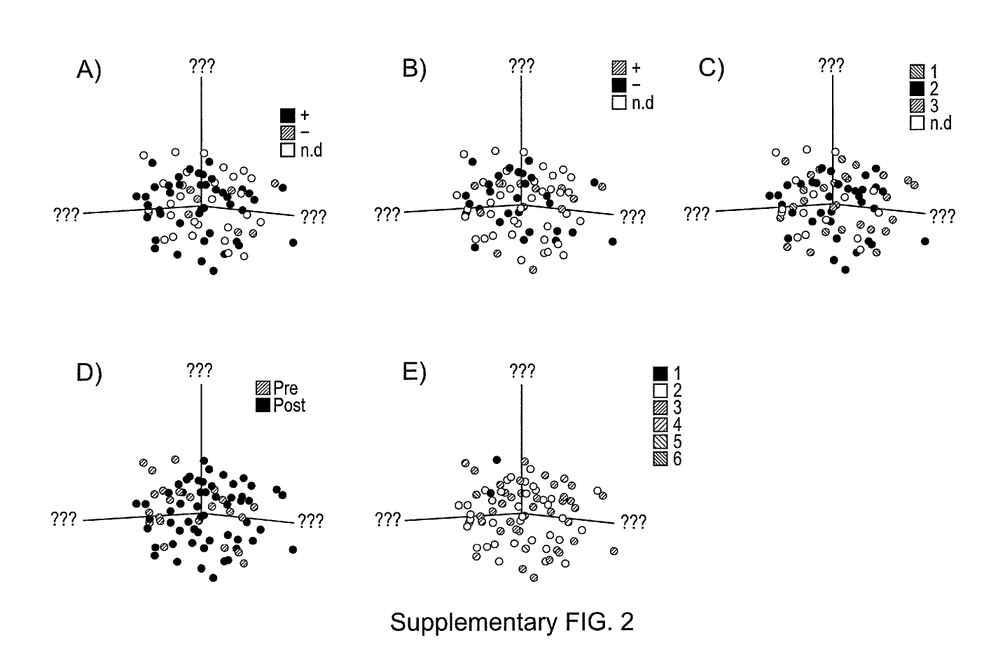

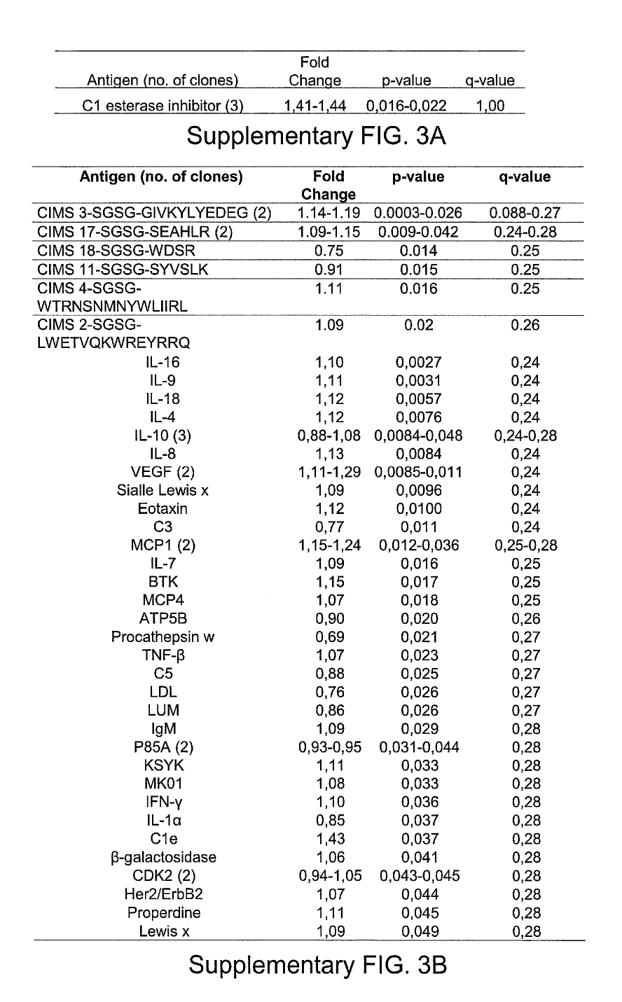

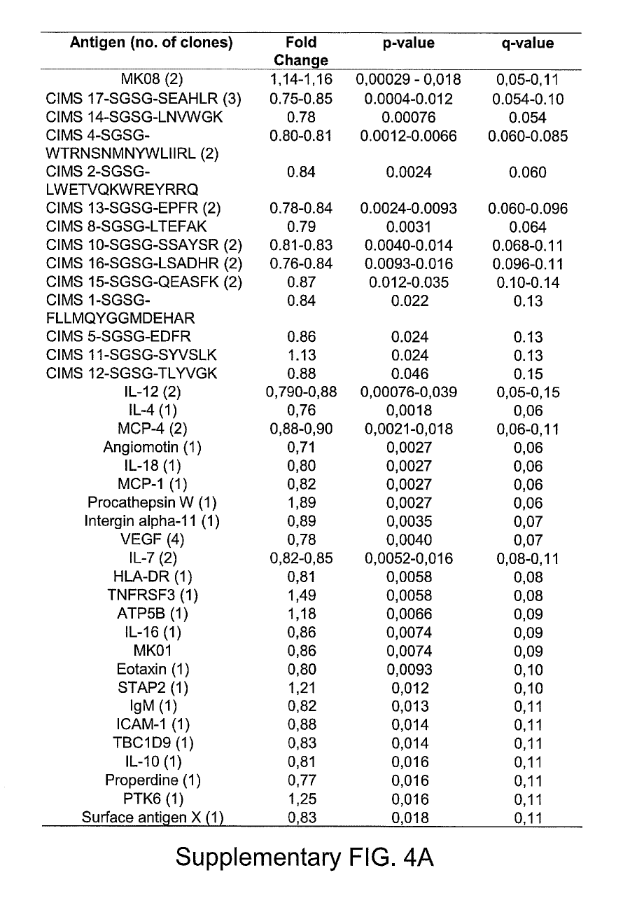

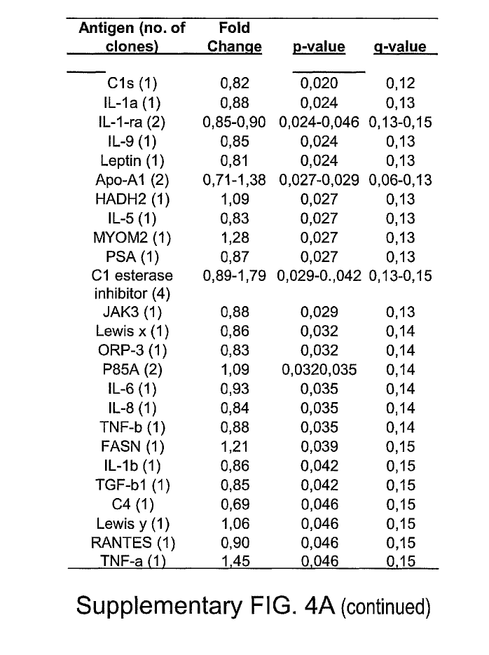

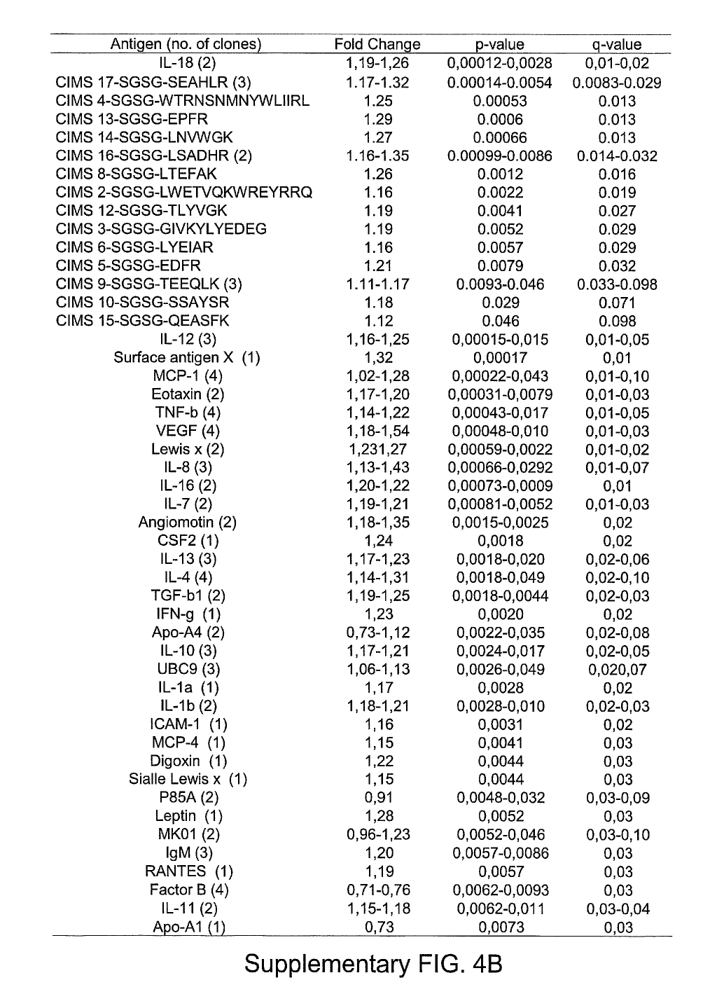

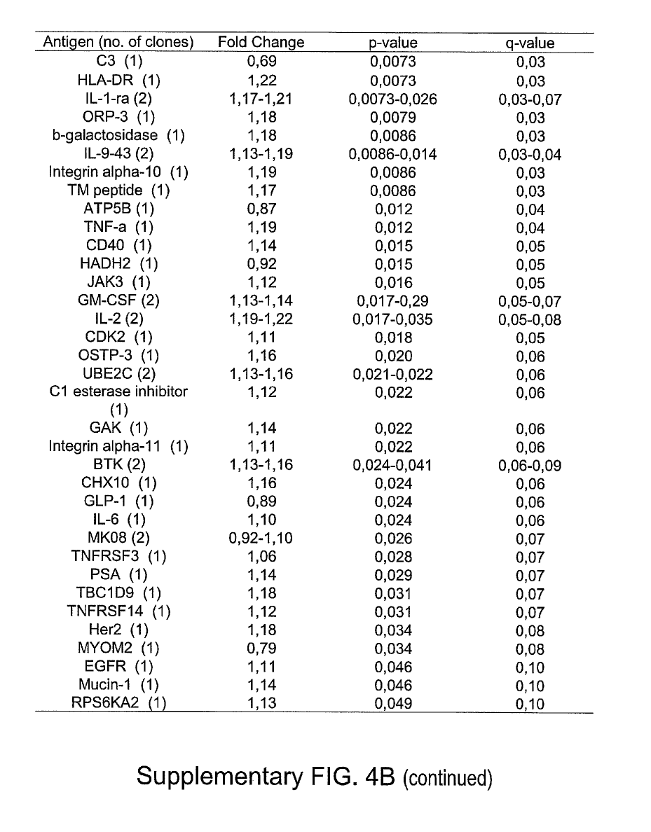

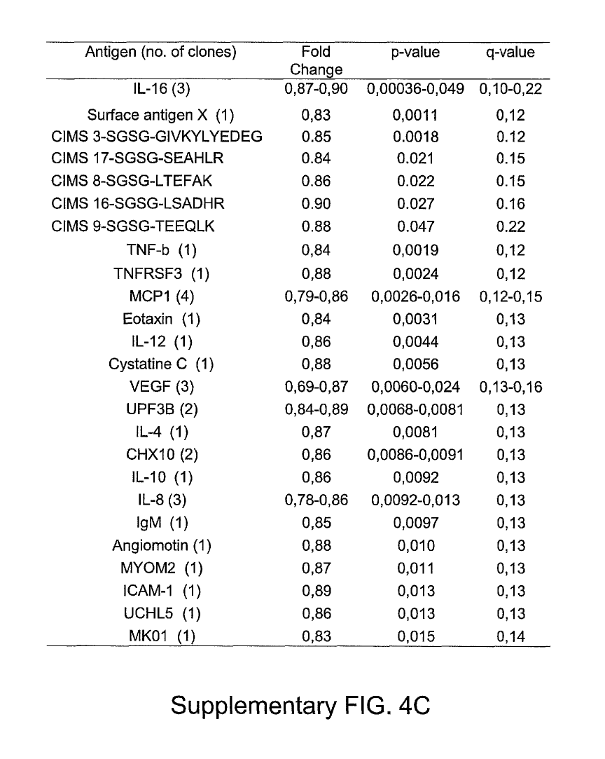

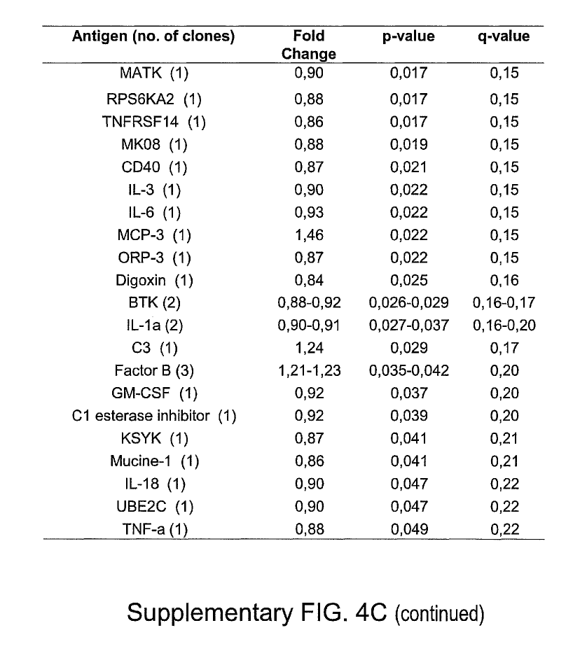

In an alternative or additional embodiment step (b) comprises or consists of measuring the presence and/or amount of 1 or more biomarker listed in FIG. 4(C), for example at least 2, 3, 4, 5, 6, 7, 8, 9, 10, 11, 12, 13, 14, 15, 16, 17 or 18 of the biomarkers listed in FIG. 4(C). Hence, the method may be indicative of whether or not the test sample is characteristic of a sample taken from an individual with breast cancer 0-26 or 26-52 weeks prior to diagnosis by conventional clinical methods.

In an alternative or additional embodiment step (b) comprises or consists of measuring the presence and/or amount of 1 or more biomarker listed in FIG. 4(D), for example at least 2, 3, 4, 5, 6, 7, 8, 9, 10, 11, 12, 13, 14, 15, 16, 17 or 18 of the biomarkers listed in FIG. 4(D). Hence, the method may be indicative of whether or not the test sample is characteristic of a sample taken from an individual with breast cancer 26-52 or 52-78 weeks prior to diagnosis by conventional clinical methods.

In an alternative or additional embodiment step (b) comprises or consists of measuring the presence and/or amount of 1 or more biomarker listed in FIG. 4(D), for example at least 2, 3, 4, 5, 6, 7, 8, 9, 10, 11, 12, 13, 14, 15, 16, 17 or 18 of the biomarkers listed in FIG. 4(D). Hence, the method may be indicative of whether or not the test sample is characteristic of a sample taken from an individual with breast cancer 52-78 or 78-104 weeks prior to diagnosis by conventional clinical methods.

In an alternative or additional embodiment step (b) comprises measuring the presence and/or amount of all of the biomarkers listed in Table A and/or Table B.

If and where there is variance between Tables A and B and the figures and other tables provided herein, the figures and other tables provided herein take precedence.

Hence, the method according to the first aspect of the invention may include or exclude measuring the presence and/or amount of AKT3. The method according to the first aspect of the invention may include or exclude measuring the presence and/or amount of Angiomotin. The method according to the first aspect of the invention may include or exclude measuring the presence and/or amount of Apo-A1. The method according to the first aspect of the invention may include or exclude measuring the presence and/or amount of Apo-A4. The method according to the first aspect of the invention may include or exclude measuring the presence and/or amount of ATPSB. The method according to the first aspect of the invention may include or exclude measuring the presence and/or amount of BTK. The method according to the first aspect of the invention may include or exclude measuring the presence and/or amount of C1 esterase inhibitor. The method according to the first aspect of the invention may include or exclude measuring the presence and/or amount of C1q. The method according to the first aspect of the invention may include or exclude measuring the presence and/or amount of C1s. The method according to the first aspect of the invention may include or exclude measuring the presence and/or amount of C3. The method according to the first aspect of the invention may include or exclude measuring the presence and/or amount of C4. The method according to the first aspect of the invention may include or exclude measuring the presence and/or amount of C5. The method according to the first aspect of the invention may include or exclude measuring the presence and/or amount of CD40. The method according to the first aspect of the invention may include or exclude measuring the presence and/or amount of CD40L. The method according to the first aspect of the invention may include or exclude measuring the presence and/or amount of CDK2. The method according to the first aspect of the invention may include or exclude measuring the presence and/or amount of CHX10. The method according to the first aspect of the invention may include or exclude measuring the presence and/or amount of CIMS—SGSG-FLLMQYGGMDEHAR. The method according to the first aspect of the invention may include or exclude measuring the presence and/or amount of CIMS—SGSG-GIVKYLYEDEG. The method according to the first aspect of the invention may include or exclude measuring the presence and/or amount of CIMS—SGSG-LWETVQKWREYRRQ. The method according to the first aspect of the invention may include or exclude measuring the presence and/or amount of CIMS—SGSG-WTRNSNMNYWLIIRL. The method according to the first aspect of the invention may include or exclude measuring the presence and/or amount of CIMS—SGSG-DFAEDK. The method according to the first aspect of the invention may include or exclude measuring the presence and/or amount of CIMS—SGSG-EDFR. The method according to the first aspect of the invention may include or exclude measuring the presence and/or amount of CIMS—SGSG-EPFR. The method according to the first aspect of the invention may include or exclude measuring the presence and/or amount of CIMS—SGSG-LNVWGK. The method according to the first aspect of the invention may include or exclude measuring the presence and/or amount of CIMS—SGSG-LSADHR. The method according to the first aspect of the invention may include or exclude measuring the presence and/or amount of CIMS—SGSG-LTEFAK. The method according to the first aspect of the invention may include or exclude measuring the presence and/or amount of CIMS—SGSG-LYEIAR. The method according to the first aspect of the invention may include or exclude measuring the presence and/or amount of CIMS—SGSG-QEASFK. The method according to the first aspect of the invention may include or exclude measuring the presence and/or amount of CIMS—SGSG-SEAHLR. The method according to the first aspect of the invention may include or exclude measuring the presence and/or amount of CIMS—SGSG-SSAYSR. The method according to the first aspect of the invention may include or exclude measuring the presence and/or amount of CIMS—SGSG-SYVSLK. The method according to the first aspect of the invention may include or exclude measuring the presence and/or amount of CIMS—SGSG-TEEQLK. The method according to the first aspect of the invention may include or exclude measuring the presence and/or amount of CIMS—SGSG-TLYVGK. The method according to the first aspect of the invention may include or exclude measuring the presence and/or amount of CIMS—SGSG-WDSR. The method according to the first aspect of the invention may include or exclude measuring the presence and/or amount of CSF2. The method according to the first aspect of the invention may include or exclude measuring the presence and/or amount of CT17. The method according to the first aspect of the invention may include or exclude measuring the presence and/or amount of Cystatine C. The method according to the first aspect of the invention may include or exclude measuring the presence and/or amount of Digoxin. The method according to the first aspect of the invention may include or exclude measuring the presence and/or amount of EGFR. The method according to the first aspect of the invention may include or exclude measuring the presence and/or amount of Eotaxin. The method according to the first aspect of the invention may include or exclude measuring the presence and/or amount of Factor B. The method according to the first aspect of the invention may include or exclude measuring the presence and/or amount of FASN. The method according to the first aspect of the invention may include or exclude measuring the presence and/or amount of GAK. The method according to the first aspect of the invention may include or exclude measuring the presence and/or amount of GLP-1. The method according to the first aspect of the invention may include or exclude measuring the presence and/or amount of GM-CSF. The method according to the first aspect of the invention may include or exclude measuring the presence and/or amount of HADH2. The method according to the first aspect of the invention may include or exclude measuring the presence and/or amount of Her2/ErbB-2. The method according to the first aspect of the invention may include or exclude measuring the presence and/or amount of HLA-DR. The method according to the first aspect of the invention may include or exclude measuring the presence and/or amount of ICAM-1. The method according to the first aspect of the invention may include or exclude measuring the presence and/or amount of IFN-γ. The method according to the first aspect of the invention may include or exclude measuring the presence and/or amount of IgM. The method according to the first aspect of the invention may include or exclude measuring the presence and/or amount of IL-10. The method according to the first aspect of the invention may include or exclude measuring the presence and/or amount of IL-11. The method according to the first aspect of the invention may include or exclude measuring the presence and/or amount of IL-12. The method according to the first aspect of the invention may include or exclude measuring the presence and/or amount of IL-13. The method according to the first aspect of the invention may include or exclude measuring the presence and/or amount of IL-16. The method according to the first aspect of the invention may include or exclude measuring the presence and/or amount of IL-18. The method according to the first aspect of the invention may include or exclude measuring the presence and/or amount of IL-1a. The method according to the first aspect of the invention may include or exclude measuring the presence and/or amount of IL-1b. The method according to the first aspect of the invention may include or exclude measuring the presence and/or amount of IL-1-ra. The method according to the first aspect of the invention may include or exclude measuring the presence and/or amount of IL-2. The method according to the first aspect of the invention may include or exclude measuring the presence and/or amount of IL-3. The method according to the first aspect of the invention may include or exclude measuring the presence and/or amount of IL-4. The method according to the first aspect of the invention may include or exclude measuring the presence and/or amount of IL-5. The method according to the first aspect of the invention may include or exclude measuring the presence and/or amount of IL-6. The method according to the first aspect of the invention may include or exclude measuring the presence and/or amount of IL-7. The method according to the first aspect of the invention may include or exclude measuring the presence and/or amount of IL-8. The method according to the first aspect of the invention may include or exclude measuring the presence and/or amount of IL-9. The method according to the first aspect of the invention may include or exclude measuring the presence and/or amount of Integrin alpha-10. The method according to the first aspect of the invention may include or exclude measuring the presence and/or amount of Integrin alpha-11. The method according to the first aspect of the invention may include or exclude measuring the presence and/or amount of JAK3. The method according to the first aspect of the invention may include or exclude measuring the presence and/or amount of Keratin19. The method according to the first aspect of the invention may include or exclude measuring the presence and/or amount of KSYK. The method according to the first aspect of the invention may include or exclude measuring the presence and/or amount of LDL. The method according to the first aspect of the invention may include or exclude measuring the presence and/or amount of Leptin. The method according to the first aspect of the invention may include or exclude measuring the presence and/or amount of Lewis x. The method according to the first aspect of the invention may include or exclude measuring the presence and/or amount of Lewis y. The method according to the first aspect of the invention may include or exclude measuring the presence and/or amount of LUM. The method according to the first aspect of the invention may include or exclude measuring the presence and/or amount of MATK. The method according to the first aspect of the invention may include or exclude measuring the presence and/or amount of MCP-1. The method according to the first aspect of the invention may include or exclude measuring the presence and/or amount of MCP-3. The method according to the first aspect of the invention may include or exclude measuring the presence and/or amount of MCP-4. The method according to the first aspect of the invention may include or exclude measuring the presence and/or amount of MK01. The method according to the first aspect of the invention may include or exclude measuring the presence and/or amount of MK08. The method according to the first aspect of the invention may include or exclude measuring the presence and/or amount of Mucin-1. The method according to the first aspect of the invention may include or exclude measuring the presence and/or amount of MYOM2. The method according to the first aspect of the invention may include or exclude measuring the presence and/or amount of ORP-3. The method according to the first aspect of the invention may include or exclude measuring the presence and/or amount of OSBPL3. The method according to the first aspect of the invention may include or exclude measuring the presence and/or amount of OSTP. The method according to the first aspect of the invention may include or exclude measuring the presence and/or amount of P85A. The method according to the first aspect of the invention may include or exclude measuring the presence and/or amount of Procathepsin W. The method according to the first aspect of the invention may include or exclude measuring the presence and/or amount of Properdine. The method according to the first aspect of the invention may include or exclude measuring the presence and/or amount of PSA. The method according to the first aspect of the invention may include or exclude measuring the presence and/or amount of PTK6. The method according to the first aspect of the invention may include or exclude measuring the presence and/or amount of PTPN1. The method according to the first aspect of the invention may include or exclude measuring the presence and/or amount of RANTES. The method according to the first aspect of the invention may include or exclude measuring the presence and/or amount of RPS6KA2. The method according to the first aspect of the invention may include or exclude measuring the presence and/or amount of Sialle Lewis x. The method according to the first aspect of the invention may include or exclude measuring the presence and/or amount of STAP2. The method according to the first aspect of the invention may include or exclude measuring the presence and/or amount of Surface antigen X. The method according to the first aspect of the invention may include or exclude measuring the presence and/or amount of TBC1D9. The method according to the first aspect of the invention may include or exclude measuring the presence and/or amount of TENS4. The method according to the first aspect of the invention may include or exclude measuring the presence and/or amount of TGF-β1. The method according to the first aspect of the invention may include or exclude measuring the presence and/or amount of TM peptide. The method according to the first aspect of the invention may include or exclude measuring the presence and/or amount of TNF-α. The method according to the first aspect of the invention may include or exclude measuring the presence and/or amount of TNF-b. The method according to the first aspect of the invention may include or exclude measuring the presence and/or amount of TNFRSF14. The method according to the first aspect of the invention may include or exclude measuring the presence and/or amount of TNFRSF3. The method according to the first aspect of the invention may include or exclude measuring the presence and/or amount of UBC9. The method according to the first aspect of the invention may include or exclude measuring the presence and/or amount of UBE2C. The method according to the first aspect of the invention may include or exclude measuring the presence and/or amount of UCHLS. The method according to the first aspect of the invention may include or exclude measuring the presence and/or amount of UPF3B. The method according to the first aspect of the invention may include or exclude measuring the presence and/or amount of VEGF. The method according to the first aspect of the invention may include or exclude measuring the presence and/or amount of β-galactosidase.

By “TM peptide” we mean a peptide derived from a 10TM protein, to which the scFv antibody construct of SEQ ID NO:1 below has specificity (wherein the CDR sequences are underlined):

[SEQ ID NO: 1]
MAEVQLLESGGGLVQPGGSLRLSCAASGFTFSSYGFHWVRQAPGKGLEWV
SLISWDGGSTYYADSVKGRFTISRDNSKNTLYLQMNSLRAEDTAVYYCAR
GTWFDPWGQGTLVTVSSGGGGSGGGGSGGGGSQSVLTQPPSASGTPGQRV
TISCSGSSSNIGNNAVNWYQQLPGTAPKLLIYRNNQRPSGVPDRFSGSKS
GTSASLAISGLRSEDEADYYCAAWDDSLSWVFGGGTKLTVLG

Hence, this scFv may be used or any antibody, or antigen binding fragment thereof, that competes with this scFv for binding to the 10TM protein. For example, the antibody, or antigen binding fragment thereof, may comprise the same CDRs as present in SEQ ID NO:1.

It will be appreciated by persons skilled in the art that such an antibody may be produced with an affinity tag (e.g. at the C-terminus) for purification purposes. For example, an affinity tag of SEQ ID NO:2 below may be utilised:

[SEQ ID NO: 2]
DYKDHDGDYKDHDIDYKDDDDKAAAHHHHHH

By “expression” we include the level or amount of a gene product such as mRNA or protein.

In an alternative or additional embodiment the method comprises the steps comprising or consisting of:
c) providing one or more control sample from:
   i. an individual not afflicted with breast cancer; and/or
   ii. an individual afflicted with breast cancer, wherein the sample was taken at a time period defined in Claims 7-37 that differs from the time period that the test sample is characteristic of;
d) determining a biomarker signature of the one or more control sample by measuring the presence and/or amount in the control sample of the one or more biomarkers measured in step (b);
wherein the presence of breast cancer is identified in the event that the presence and/or amount in the test sample of the one or more biomarkers measured in step (b) is different from the presence and/or amount in the control sample of the one or more biomarkers measured in step (d).

By “is different to the presence and/or amount in a control sample” we mean the presence and/or amount of the one or more biomarker in the test sample differs from that of the one or more control sample (or to predefined reference values representing the same). Preferably the presence and/or amount is no more than 40% of that of the one or more negative control sample, for example, no more than 39%, 38%, 37%, 36%, 35%, 34%, 33%, 32%, 31%, 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, 1% or 0%.

In an alternative or additional embodiment the presence and/or amount in the test sample of the one or more biomarker measured in step (b) is significantly different (i.e., statistically significantly different) from the presence and/or amount of the one or more biomarker measured in step (d) or the predetermined reference values. For example, as discussed in the accompanying Examples, significant difference between the presence and/or amount of a particular biomarker in the test and control samples may be classified as those where p<0.05 (for example, where p<0.04, p<0.03, p<0.02 or where p<0.01).

By “the time period that the test sample is characteristic of” we include the time period prior to breast cancer diagnosis by conventional clinical methods.

In an alternative or additional embodiment the method comprises the steps comprising or consisting of:
e) providing one or more control sample from;
   i. an individual afflicted with breast cancer (i.e., a positive control); and/or
   ii. an individual afflicted with breast cancer, wherein the sample was taken at a time period defined in Claims 7-37 that corresponds to the time period that the test sample is characteristic of;
f) determining a biomarker signature of the control sample by measuring the presence and/or amount in the control sample of the one or more biomarkers measured in step (b);
wherein the presence of breast cancer is identified in the event that the presence and/or amount in the test sample of the one or more biomarkers measured in step (b) corresponds to the presence and/or amount in the control sample of the one or more biomarkers measured in step (f).

By “corresponds to the presence and/or amount in a control sample” we mean the presence and/or amount is identical to that of a positive control sample; or closer to that of one or more positive control sample than to one or more negative control sample (or to predefined reference values representing the same). Preferably the presence and/or amount is at least 60% of that of the control sample comprising or consisting breast cancer cells of a first histological grade, for example, at least 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an alternative or additional embodiment the control samples comprise one or more sample taken from each of the time periods defined in preceding embodiments (e.g., 0-52 weeks from diagnosis or 52-104 weeks from diagnosis etc). The control samples may comprise one or more sample taken from each of the time periods defined in in preceding embodiments.

In an alternative or additional embodiment the one or more control samples are age-, weight and/or sex-matched for the individual to be tested. In other words, the healthy individual is approximately the same age (e.g., within 1, 2, 3, 4 or 5 years), approximately the same weight (e.g., within 12%, 11%, 10%, 9%, 8%, 7%, 6%, 5%, 4%, 3%, 2%, 1%, 0.5% or 0.1%) and is the same sex as the individual to be tested.

In an alternative or additional embodiment the presence and/or amount in the test sample of the one or more biomarkers measured in step (b) are compared against predetermined reference values.

In an alternative or additional embodiment the presence and/or amount in the test sample of the one or more biomarker measured in step (b) is significantly correlated with/similar to (i.e., statistically significantly correlated/similar) the presence and/or amount of the one or more biomarker measured in step (f) or the predetermined reference values. For example, as discussed in the accompanying Examples, significant difference between the presence and/or amount of a particular biomarker in the test and control samples may be classified as those where p<0.05 (for example, where p<0.04, p<0.03, p<0.02 or where p<0.01).

In an alternative or additional embodiment the individual from which the one or more control sample was obtained was not, at the time the sample was obtained, afflicted with breast abscess, breast fibroadenoma, fibroadenoma, fibrocystic breast disease, fibrocystic breasts, gynecomastia, mastalgia and/or mastitis.

In an alternative or additional embodiment the individual from which the one or more control sample was obtained was not, at the time the sample was obtained, afflicted with any disease or condition of the breast (or, for control samples from breast cancer patients, any other disease or condition of the breast). Preferably, the individual from which the one or more control sample was obtained was not, at the time the sample was obtained, afflicted with any disease or condition (or, for control samples from breast cancer patients, any disease or condition other than breast cancer). Hence, the individual not afflicted with breast cancer may be a healthy individual.

In an alternative or additional embodiment the individual afflicted with breast cancer is afflicted with a breast cancer selected from the group consisting of ductal carcinoma in situ (DCIS), lobular carcinoma in situ (LCIS), invasive ductal breast cancer, invasive lobular breast cancer, Inflammatory breast cancer, medullary breast cancer, mucinous (mucoid or colloid) breast cancer, tubular breast cancer, adenoid cystic carcinoma of the breast (cribriform breast cancer), metaplastic breast cancer, angiosarcoma of the breast, lymphoma of the breast, basal type breast cancer, malignant phyllodes or cystosarcoma phyllodes and papillary breast cancer. Hence, the breast cancer may be invasive ductal breast cancer.

In an alternative or additional embodiment the method according to any one of the preceding embodiments is repeated and, preferably, in step (a), the sample to be tested is taken at different time to the previous method repetition. Preferably, the method is repeated using a test sample taken at a different time period to the previous test sample(s) used, for example, the method maybe repeated using a test sample taken between 1 day to 104 weeks to the previous test sample(s) used, for example, between 1 week to 100 weeks, 1 week to 90 weeks, 1 week to 80 weeks, 1 week to 70 weeks, 1 week to 60 weeks, 1 week to 50 weeks, 1 week to 40 weeks, 1 week to 30 weeks, 1 week to 20 weeks, 1 week to 10 weeks, 1 week to 9 weeks, 1 week to 8 weeks, 1 week to 7 weeks, 1 week to 6 weeks, 1 week to 5 weeks, 1 week to 4 weeks, 1 week to 3 weeks, or 1 week to 2 weeks.

Alternatively, the method may be repeated using a test sample taken every period from the group consisting of: 1 day, 2 days, 3 day, 4 days, 5 days, 6 days, 7 days, 10 days, 2 weeks, 3 weeks, 4 weeks, 5 weeks, 6 weeks, 7 weeks, 8 weeks, 9 weeks, 10 weeks, 15 weeks, 20 weeks, 25 weeks, 30 weeks, 35 weeks, 40 weeks, 45 weeks, 50 weeks, 55 weeks, 60 weeks, 65 weeks, 70 weeks, 75 weeks, 80 weeks, 85 weeks, 90 weeks, 95 weeks, 100 weeks, 104, weeks, 105 weeks, 110 weeks, 115 weeks, 120 weeks, 125 weeks and 130 weeks.

In an alternative or additional embodiment method is repeated at least once, for example, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23, 24 times or 25 times.

In an alternative or additional embodiment the method is repeated continuously.

In an alternative or additional embodiment the method is repeated until diagnosis of breast cancer in the individual using conventional clinical methods.

In an alternative or additional embodiment each repetition uses test sample taken from the same individual.

In an alternative or additional embodiment step (b) comprises measuring the expression of the protein or polypeptide of the one or more biomarker(s).

Methods of detecting and/or measuring the concentration of protein and/or nucleic acid are well known to those skilled in the art, see for example Sambrook and Russell, 2001, Cold Spring Harbor Laboratory Press.

Preferred methods for detection and/or measurement of protein include Western blot, North-Western blot, immunosorbent assays (ELISA), antibody microarray, tissue microarray (TMA), immunoprecipitation, in situ hybridisation and other immunohistochemistry techniques, radioimmunoassay (RIA), immunoradiometric assays (IRMA) and immunoenzymatic assays (IEMA), including sandwich assays using monoclonal and/or polyclonal antibodies. Exemplary sandwich assays are described by David et al., in U.S. Pat. Nos. 4,376,110 and 4,486,530, hereby incorporated by reference. Antibody staining of cells on slides may be used in methods well known in cytology laboratory diagnostic tests, as well known to those skilled in the art.

Typically, ELISA involves the use of enzymes which give a coloured reaction product, usually in solid phase assays. Enzymes such as horseradish peroxidase and phosphatase have been widely employed. A way of amplifying the phosphatase reaction is to use NADP as a substrate to generate NAD which now acts as a coenzyme for a second enzyme system. Pyrophosphatase from Escherichia coli provides a good conjugate because the enzyme is not present in tissues, is stable and gives a good reaction colour. Chemi-luminescent systems based on enzymes such as luciferase can also be used.

Conjugation with the vitamin biotin is frequently used since this can readily be detected by its reaction with enzyme-linked avidin or streptavidin to which it binds with great specificity and affinity.

Preferred methods for detection and/or measurement of nucleic acid (e.g. mRNA) include southern blot, northern blot, polymerase chain reaction (PCR), reverse transcriptase PCR (RT-PCR), quantitative real-time PCR (qRT-PCR), nanoarray, microarray, macroarray, autoradiography and in situ hybridisation.

In an alternative or additional embodiment step (b), (d) and/or step (f) is performed using one or more first binding agent capable of binding to a biomarker listed in Table A or Table B.

In an alternative or additional embodiment the first binding agent comprises or consists of an antibody or an antigen-binding fragment thereof.

The term “antibody” includes any synthetic antibodies, recombinant antibodies or antibody hybrids, such as but not limited to, a single-chain antibody molecule produced by phage-display of immunoglobulin light and/or heavy chain variable and/or constant regions, or other immunointeractive molecules capable of binding to an antigen in an immunoassay format that is known to those skilled in the art. We also include the use of antibody-like binding agents, such as affibodies and aptamers.

A general review of the techniques involved in the synthesis of antibody fragments which retain their specific binding sites is to be found in Winter & Milstein (1991) Nature 349, 293-299.

Additionally, or alternatively, one or more of the first binding molecules may be an aptamer (see Collett et al., 2005, Methods 37:4-15).

Molecular libraries such as antibody libraries (Clackson et al, 1991, Nature 352, 624-628; Marks et al, 1991, J Mol Biol 222(3): 581-97), peptide libraries (Smith, 1985, Science 228(4705): 1315-7), expressed cDNA libraries (Santi et al (2000) J Mol Biol 296(2): 497-508), libraries on other scaffolds than the antibody framework such as affibodies (Gunneriusson et al, 1999, Appl Environ Microbiol 65(9): 4134-40) or libraries based on aptamers (Kenan et al, 1999, Methods Mol Biol 118, 217-31) may be used as a source from which binding molecules that are specific for a given motif are selected for use in the methods of the invention.

The molecular libraries may be expressed in vivo in prokaryotic cells (Clackson et al, 1991, op. cit.; Marks et al, 1991, op. cit.) or eukaryotic cells (Kieke et al, 1999, Proc Natl Acad Sci USA, 96(10):5651-6) or may be expressed in vitro without involvement of cells (Hanes & Pluckthun, 1997, Proc Natl Acad Sci USA 94(10):4937-42; He & Taussig, 1997, Nucleic Acids Res 25(24):5132-4; Nemoto et al, 1997, FEBS Lett, 414(2):405-8).

In cases when protein based libraries are used, the genes encoding the libraries of potential binding molecules are often packaged in viruses and the potential binding molecule displayed at the surface of the virus (Clackson et al, 1991, supra; Marks et al, 1991, supra; Smith, 1985, supra).

Perhaps the most commonly used display system is filamentous bacteriophage displaying antibody fragments at their surfaces, the antibody fragments being expressed as a fusion to the minor coat protein of the bacteriophage (Clackson et al, 1991, supra; Marks et al, 1991, supra). However, other suitable systems for display include using other viruses (EP 39578), bacteria (Gunneriusson et al, 1999, supra; Daugherty et al, 1998, Protein Eng 11(9):825-32; Daugherty et al, 1999, Protein Eng 12(7):613-21), and yeast (Shusta et al, 1999, J Mol Biol 292(5): 949-56).

In addition, display systems have been developed utilising linkage of the polypeptide product to its encoding mRNA in so-called ribosome display systems (Hanes & Pluckthun, 1997, supra; He & Taussig, 1997, supra; Nemoto et al, 1997, supra), or alternatively linkage of the polypeptide product to the encoding DNA (see U.S. Pat. No. 5,856,090 and WO 98/37186).

The variable heavy (VH) and variable light (VL) domains of the antibody are involved in antigen recognition, a fact first recognised by early protease digestion experiments. Further confirmation was found by “humanisation” of rodent antibodies. Variable domains of rodent origin may be fused to constant domains of human origin such that the resultant antibody retains the antigenic specificity of the rodent parented antibody (Morrison et al (1984) Proc. Natl. Acad. Sci. USA 81, 6851-6855).

That antigenic specificity is conferred by variable domains and is independent of the constant domains is known from experiments involving the bacterial expression of antibody fragments, all containing one or more variable domains. These molecules include Fab-like molecules (Better et al (1988) Science 240, 1041); Fv molecules (Skerra et al (1988) Science 240, 1038); single-chain Fv (ScFv) molecules where the VH and VL partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544). A general review of the techniques involved in the synthesis of antibody fragments which retain their specific binding sites is to be found in Winter & Milstein (1991) Nature 349, 293-299.

The antibody or antigen-binding fragment may be selected from the group consisting of intact antibodies, Fv fragments (e.g. single chain Fv and disulphide-bonded Fv), Fab-like fragments (e.g. Fab fragments, Fab′ fragments and F(ab)₂ fragments), single variable domains (e.g. VH and VL domains) and domain antibodies (dAbs, including single and dual formats [i.e. dAb-linker-dAb]). Preferably, the antibody or antigen-binding fragment is a single chain Fv (scFv).

The one or more binding moieties may alternatively comprise or consist of an antibody-like binding agent, for example an affibody or aptamer.

By “scFv molecules” we mean molecules wherein the VH and VL partner domains are linked via a flexible oligopeptide.

The advantages of using antibody fragments, rather than whole antibodies, are several-fold. The smaller size of the fragments may lead to improved pharmacological properties, such as better penetration of solid tissue. Effector functions of whole antibodies, such as complement binding, are removed. Fab, Fv, ScFv and dAb antibody fragments can all be expressed in and secreted from E. coli, thus allowing the facile production of large amounts of the said fragments.

Whole antibodies, and F(ab′)₂ fragments are “bivalent”. By “bivalent” we mean that the said antibodies and F(ab′)₂ fragments have two antigen combining sites. In contrast, Fab, Fv, ScFv and dAb fragments are monovalent, having only one antigen combining sites.

The antibodies may be monoclonal or polyclonal. Suitable monoclonal antibodies may be prepared by known techniques, for example those disclosed in “Monoclonal Antibodies: A manual of techniques”, H Zola (CRC Press, 1988) and in “Monoclonal Hybridoma Antibodies: Techniques and applications”, J G R Hurrell (CRC Press, 1982), both of which are incorporated herein by reference.

When potential binding molecules are selected from libraries, one or more selector peptides having defined motifs are usually employed. Amino acid residues that provide structure, decreasing flexibility in the peptide or charged, polar or hydrophobic side chains allowing interaction with the binding molecule may be used in the design of motifs for selector peptides. For example:
(i) Proline may stabilise a peptide structure as its side chain is bound both to the alpha carbon as well as the nitrogen;
(ii) Phenylalanine, tyrosine and tryptophan have aromatic side chains and are highly hydrophobic, whereas leucine and isoleucine have aliphatic side chains and are also hydrophobic;
(iii) Lysine, arginine and histidine have basic side chains and will be positively charged at neutral pH, whereas aspartate and glutamate have acidic side chains and will be negatively charged at neutral pH;
(iv) Asparagine and glutamine are neutral at neutral pH but contain a amide group which may participate in hydrogen bonds;
(v) Serine, threonine and tyrosine side chains contain hydroxyl groups, which may participate in hydrogen bonds.

Typically, selection of binding molecules may involve the use of array technologies and systems to analyse binding to spots corresponding to types of binding molecules.

In an alternative or additional embodiment the antibody or antigen-binding fragment thereof is a recombinant antibody or antigen-binding fragment thereof.

Hence, preferably the antibody or fragment thereof is a monoclonal antibody or fragment thereof. Preferably the antibody or antigen-binding fragment is selected from the group consisting of intact antibodies, Fv fragments (e.g. single chain Fv and disulphide-bonded Fv), Fab-like fragments (e.g. Fab fragments, Fab′ fragments and F(ab)₂ fragments), single variable domains (e.g. VH and VL domains) and domain antibodies (dAbs, including single and dual formats [i.e. dAb-linker-dAb]). Hence, the antibody or antigen-binding fragment may be a single chain Fv (scFv). Alternatively, the one or more binding moieties comprise or consist of an antibody-like binding agent, for example an affibody or aptamer.

In an alternative or additional embodiment the antibody or antigen-binding fragment thereof is selected from the group consisting of: scFv; Fab; a binding domain of an immunoglobulin molecule.

In an alternative or additional embodiment the first binding agent is immobilised on a surface.

In an alternative or additional embodiment the one or more biomarkers in the test sample are labelled with a detectable moiety.

By a “detectable moiety” we include a moiety which permits its presence and/or relative amount and/or location (for example, the location on an array) to be determined, either directly or indirectly.

Suitable detectable moieties are well known in the art.

For example, the detectable moiety may be a fluorescent and/or luminescent and/or chemiluminescent moiety which, when exposed to specific conditions, may be detected. Such a fluorescent moiety may need to be exposed to radiation (i.e. light) at a specific wavelength and intensity to cause excitation of the fluorescent moiety, thereby enabling it to emit detectable fluorescence at a specific wavelength that may be detected.

Alternatively, the detectable moiety may be an enzyme which is capable of converting a (preferably undetectable) substrate into a detectable product that can be visualised and/or detected. Examples of suitable enzymes are discussed in more detail below in relation to, for example, ELISA assays.

Hence, the detectable moiety may be selected from the group consisting of: a fluorescent moiety; a luminescent moiety; a chemiluminescent moiety; a radioactive moiety (for example, a radioactive atom); or an enzymatic moiety. Preferably, the detectable moiety comprises or consists of a radioactive atom. The radioactive atom may be selected from the group consisting of technetium-99m, iodine-123, iodine-125, iodine-131, indium-111, fluorine-19, carbon-13, nitrogen-15, oxygen-17, phosphorus-32, sulphur-35, deuterium, tritium, rhenium-186, rhenium-188 and yttrium-90.

Clearly, the agent to be detected (such as, for example, the one or more biomarkers in the test sample and/or control sample described herein and/or an antibody molecule for use in detecting a selected protein) must have sufficient of the appropriate atomic isotopes in order for the detectable moiety to be readily detectable.

In an alternative preferred embodiment, the detectable moiety of the binding moiety is a fluorescent moiety.

The radio- or other labels may be incorporated into the biomarkers present in the samples of the methods of the invention and/or the binding moieties of the invention in known ways. For example, if the binding agent is a polypeptide it may be biosynthesised or may be synthesised by chemical amino acid synthesis using suitable amino acid precursors involving, for example, fluorine-19 in place of hydrogen. Labels such as ^{99m}Tc, ¹²³I, ¹⁸⁶Rh, ¹⁸⁸Rh and ¹¹¹In can, for example, be attached via cysteine residues in the binding moiety. Yttrium-90 can be attached via a lysine residue. The IODOGEN method (Fraker et al (1978) Biochem. Biophys. Res. Comm. 80, 49-57) can be used to incorporate ¹²³I. Reference (“Monoclonal Antibodies in Immunoscintigraphy”, J-F Chatal, CRC Press, 1989) describes other methods in detail. Methods for conjugating other detectable moieties (such as enzymatic, fluorescent, luminescent, chemiluminescent or radioactive moieties) to proteins are well known in the art.

It will be appreciated by persons skilled in the art that biomarkers in the sample(s) to be tested may be labelled with a moiety which indirectly assists with determining the presence, amount and/or location of said proteins. Thus, the moiety may constitute one component of a multicomponent detectable moiety. For example, the biomarkers in the sample(s) to be tested may be labelled with biotin, which allows their subsequent detection using streptavidin fused or otherwise joined to a detectable label.

Alternatively, the detectable moiety of the binding moiety may be a fluorescent moiety.

In an alternative or additional embodiment the one or more biomarkers in the control sample(s) are labelled with a detectable moiety.

In an alternative or additional embodiment the detectable moiety is selected from the group consisting of: a fluorescent moiety; a luminescent moiety; a chemiluminescent moiety; a radioactive moiety; an enzymatic moiety. Preferably the detectable moiety is biotin.

In an alternative or additional embodiment step (b), (d) and/or step (f) is performed using an assay comprising a second binding agent capable of binding to the one or more biomarkers, the second binding agent comprising a detectable moiety.

In an alternative or additional embodiment the second binding agent comprises or consists of an antibody or an antigen-binding fragment thereof. In an alternative or additional embodiment the antibody or antigen-binding fragment thereof is a recombinant antibody or antigen-binding fragment thereof. In an alternative or additional embodiment the antibody or antigen-binding fragment thereof is selected from the group consisting of: scFv; Fab; a binding domain of an immunoglobulin molecule. In an alternative or additional embodiment the detectable moiety is selected from the group consisting of: a fluorescent moiety; a luminescent moiety; a chemiluminescent moiety; a radioactive moiety; an enzymatic moiety. In an alternative or additional embodiment the detectable moiety is fluorescent moiety (for example an Alexa Fluor dye, e.g. Alexa647).

In an alternative or additional embodiment the method comprises or consists of an ELISA (Enzyme Linked Immunosorbent Assay).

The method of the first aspect of the invention may be performed using a support vector machine (SVM), such as those available from http://cran.r-project.org/web/packages/e1071/index.html (e.g. e1071 1.5-24). However, any other suitable means may also be used. SVMs may also be used to determine the ROC AUCs of biomarker signatures comprising or consisting of one or more Table A or B biomarkers as defined herein.

Support vector machines (SVMs) are a set of related supervised learning methods used for classification and regression. Given a set of training examples, each marked as belonging to one of two categories, an SVM training algorithm builds a model that predicts whether a new example falls into one category or the other. Intuitively, an SVM model is a representation of the examples as points in space, mapped so that the examples of the separate categories are divided by a clear gap that is as wide as possible. New examples are then mapped into that same space and predicted to belong to a category based on which side of the gap they fall on.

More formally, a support vector machine constructs a hyperplane or set of hyperplanes in a high or infinite dimensional space, which can be used for classification, regression or other tasks. Intuitively, a good separation is achieved by the hyperplane that has the largest distance to the nearest training datapoints of any class (so-called functional margin), since in general the larger the margin the lower the generalization error of the classifier. For more information on SVMs, see for example, Burges, 1998, Data Mining and Knowledge Discovery, 2:121-167.

In one embodiment of the invention, the SVM is ‘trained’ prior to performing the methods of the invention using biomarker profiles of known agents (namely, breast cancer cells of known histological grade or breast cancer cells from breast cancer patients with known distant metastasis-free survival). By running such training samples, the SVM is able to learn what biomarker profiles are associated with particular characteristics. Once the training process is complete, the SVM is then able whether or not the biomarker sample tested is from a particular breast cancer sample type (i.e., a particular breast cancer-associated disease state).

However, this training procedure can be by-passed by pre-programming the SVM with the necessary training parameters. For example, cells belonging to a particular breast cancer-associated disease state can be identified according to the known SVM parameters using the SVM algorithm detailed in Table 4, based on the measurement of the biomarkers listed in Table 1 using the values and/or regulation patterns detailed therein.

It will be appreciated by skilled persons that suitable SVM parameters can be determined for any combination of the biomarkers listed Table A or B by training an SVM machine with the appropriate selection of data (i.e. biomarker measurements from cells of known histological grade and/or cells from individuals with known metastasis-free survival times).

Alternatively, the data provided in the present figures and tables may be used to determine a particular breast cancer-associated disease state according to any other suitable statistical method known in the art, such as Principal Component Analysis (PCA) and other multivariate statistical analyses (e.g., backward stepwise logistic regression model). For a review of multivariate statistical analysis see, for example, Schervish, Mark J. (November 1987). “A Review of Multivariate Analysis”. Statistical Science 2 (4): 396-413 which is incorporated herein by reference.

Preferably, the method of the invention has an accuracy of at least 65%, for example 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% accuracy.

Preferably, the method of the invention has a sensitivity of at least 65%, for example 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% sensitivity.

Preferably, the method of the invention has a specificity of at least 65%, for example 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100% specificity.

By “accuracy” we mean the proportion of correct outcomes of a method, by “sensitivity” we mean the proportion of all positive samples that are correctly classified as positives, and by “specificity” we mean the proportion of all negative samples that are correctly classified as negatives.

In an alternative or additional embodiment the predicative accuracy of the method, as determined by an ROC AUC value, is at least 0.50, for example at least 0.55, 0.60, 0.65, 0.70, 0.75, 0.80, 0.85, 0.90, 0.95, 0.96, 0.97, 0.98 or at least 0.99 (e.g., 1).

In an alternative or additional embodiment the predicative accuracy of the method, as determined by an ROC AUC value, is at least 0.70.

In an alternative or additional embodiment step (b), (d) and/or step (f) is performed using an array. In an alternative or additional embodiment the array is a bead-based array. In an alternative or additional embodiment the array is a surface-based array.

In an alternative or additional embodiment the array is selected from the group consisting of: macroarray; microarray; nanoarray.

In an alternative or additional embodiment the method comprises:
(i) labelling biomarkers present in the sample with biotin;
(ii) contacting the biotin-labelled proteins with an array comprising a plurality of scFv immobilised at discrete locations on its surface, the scFv having specificity for one or more of the proteins in Table A or B;
(iii) contacting the immobilised scFv with a streptavidin conjugate comprising a fluorescent dye; and
(iv) detecting the presence of the dye at discrete locations on the array surface
wherein the expression of the dye on the array surface is indicative of the expression of a biomarker from Table III in the sample.

In an alternative or additional embodiment step (b), (d) and/or (f) comprises measuring the expression of a nucleic acid molecule encoding the one or more biomarkers.

The nucleic acid molecule may be a cDNA molecule or an mRNA molecule. Preferably the nucleic acid molecule is an mRNA molecule. Also preferably the nucleic acid molecule is a cDNA molecule.

Hence, measuring the expression of the one or more biomarker(s) in step (b) may be performed using a method selected from the group consisting of Southern hybridisation, Northern hybridisation, polymerase chain reaction (PCR), reverse transcriptase PCR (RT-PCR), quantitative real-time PCR (qRT-PCR), nanoarray, microarray, macroarray, autoradiography and in situ hybridisation. Preferably measuring the expression of the one or more biomarker(s) in step (b) is determined using a DNA microarray. Hence, the method may comprise or consist of measuring the expression of the one or more biomarker(s) in step (b) using one or more binding moiety, each capable of binding selectively to a nucleic acid molecule encoding one of the biomarkers identified in Table A or B.

In an alternative or additional embodiment step the one or more binding moieties each comprise or consist of a nucleic acid molecule such as DNA, RNA, PNA, LNA, GNA, TNA or PMO (preferably DNA). Preferably the one or more binding moieties are 5 to 100 nucleotides in length. More preferably, the one or more nucleic acid molecules are 15 to 35 nucleotides in length. The binding moiety may comprise a detectable moiety.

Suitable binding agents (also referred to as binding molecules) may be selected or screened from a library based on their ability to bind a given nucleic acid, protein or amino acid motif.

In an alternative or additional embodiment measuring the expression of the one or more biomarker(s) in step (b), (d) and/or (f) is performed using one or more binding moieties, each individually capable of binding selectively to a nucleic acid molecule encoding one of the biomarkers identified in Table A or Table B.

In an alternative or additional embodiment wherein the binding moiety comprises a detectable moiety. In an alternative or additional embodiment the detectable moiety is selected from the group consisting of: a fluorescent moiety; a luminescent moiety; a chemiluminescent moiety; a radioactive moiety (for example, a radioactive atom); or an enzymatic moiety. In an alternative or additional embodiment the detectable moiety comprises or consists of a radioactive atom. In an alternative or additional embodiment the radioactive atom is selected from the group consisting of technetium-99m, iodine-123, iodine-125, iodine-131, indium-111, fluorine-19, carbon-13, nitrogen-15, oxygen-17, phosphorus-32, sulphur-35, deuterium, tritium, rhenium-186, rhenium-188 and yttrium-90. In an alternative or additional embodiment the detectable moiety of the binding moiety is a fluorescent moiety.

In an alternative or additional embodiment the sample provided in step (a), (c) and/or (e) is selected from the group consisting of unfractionated blood, plasma, serum, tissue fluid, breast tissue, milk, bile and urine.

In an alternative or additional embodiment the sample provided in step (a), (c) and/or (e) is selected from the group consisting of unfractionated blood, plasma and serum.

In an alternative or additional embodiment the sample provided in step (a), (c) and/or (e) is serum.

In an alternative or additional embodiment in the event that the individual is diagnosed with breast cancer, the method comprises recording the diagnosis on a physical or electronic data carrier (i.e., physical or electronic file).

In an alternative or additional embodiment in the event that the individual is diagnosed with breast cancer, the method comprises the step of:
(g) providing the individual with breast cancer therapy.

As noted above, in the event that the individual is not diagnosed with breast cancer, they may be subjected to further monitoring for breast cancer (for example, using the method of the present invention).

In an alternative or additional embodiment the breast cancer therapy is selected from the group consisting of surgery, chemotherapy, immunotherapy, chemoimmunotherapy and thermochemotherapy (e.g., AC chemotherapy; Capecitabine and docetaxel chemotherapy (Taxotere®); CMF chemotherapy; Cyclophosphamide; EC chemotherapy; ECF chemotherapy; E-CMF chemotherapy (Epi-CMF); Eribulin (Halaven®); FEC chemotherapy; FEC-T chemotherapy; Fluorouracil (5FU); GemCarbo chemotherapy; Gemcitabine (Gemzar 0); Gemcitabine and cisplatin chemotherapy (GemCis or GemCisplat); GemTaxol chemotherapy; Idarubicin (Zavedos®); Liposomal doxorubicin (DaunoXome®); Mitomycin (Mitomycin C Kyowa®); Mitoxantrone; MM chemotherapy; MMM chemotherapy; Paclitaxel (Taxol®); TAC chemotherapy; Taxotere and cyclophosphamide (TC) chemotherapy; Vinblastine (Velbe®); Vincristine (Oncovin®); Vindesine (Eldisine®); and Vinorelbine (Navelbine®)).

Hence, the method comprises treating the patient according to (a) the presence of breast cancer and, optionally, (b) whether or not the test sample is characteristic of a sample taken from an individual with breast cancer X weeks prior to diagnosis by conventional clinical methods (wherein “X” means any number or number range, in particular, a range defined in the first aspect of the invention (e.g., 0-104 weeks pre-diagnosis, 0-52 weeks pre-diagnosis, 52-104 weeks pre-diagnosis)).

For example, a more aggressive treatment may be provided for later-stage/more developed breast cancers. Suitable therapeutic approaches can be determined by the skilled person according to the prevailing guidance at the time, for example, see NICE Clinical Guideline 80 “Early and locally advanced breast cancer: Diagnosis and treatment”, (available here: http://www.nice.org.uk/nicemedia/pdf/CG80NICEGuideline.pdf.) which is incorporated herein by reference.

Accordingly, the present invention comprises an antineoplastic agent for use in treating breast cancer wherein the dosage regime is determined based on the results of the method of the first aspect of the invention.

The present invention comprises the use of an antineoplastic agent in treating breast cancer wherein the dosage regime is determined based on the results of the method of the first aspect of the invention.

The present invention comprises the use of an antineoplastic agent in the manufacture of a medicament for treating breast cancer wherein the dosage regime is determined based on the results of the method of the first aspect of the invention.

The present invention comprises a method of treating breast cancer comprising providing a sufficient amount of an antineoplastic agent wherein the amount of antineoplastic agent sufficient to treat the breast cancer is determined based on the results of the method of the first aspect of the invention.

In one embodiment, the antineoplastic agent is an alkylating agent (ATC code L01a), an antimetabolite (ATC code L01b), a plant alkaloid or other natural product (ATC code L01c), a cytotoxic antibiotic or a related substance (ATC code L01d), or another antineoplastic agents (ATC code L01x).

Hence, in one embodiment the antineoplastic agent is an alkylating agent selected from the group consisting of a nitrogen mustard analogue (for example cyclophosphamide, chlorambucil, melphalan, chlormethine, ifosfamide, trofosfamide, prednimustine or bendamustine) an alkyl sulfonate (for example busulfan, treosulfan, or mannosulfan) an ethylene imine (for example thiotepa, triaziquone or carboquone) a nitrosourea (for example carmustine, lomustine, semustine, streptozocin, fotemustine, nimustine or ranimustine) an epoxides (for example etoglucid) or another alkylating agent (ATC code L01ax, for example mitobronitol, pipobroman, temozolomide or dacarbazine).

In a another embodiment the antineoplastic agent is an antimetabolite selected from the group consisting of a folic acid analogue (for example methotrexate, raltitrexed, pemetrexed or pralatrexate), a purine analogue (for example mercaptopurine, tioguanine, cladribine, fludarabine, clofarabine or nelarabine) or a pyrimidine analogue (for example cytarabine, fluorouracil, tegafur, carmofur, gemcitabine, capecitabine, azacitidine or decitabine).

In a still further embodiment the antineoplastic agent is a plant alkaloid or other natural product selected from the group consisting of a vinca alkaloid or a vinca alkaloid analogue (for example vinblastine, vincristine, vindesine, vinorelbine or vinflunine), a podophyllotoxin derivative (for example etoposide or teniposide) a colchicine derivative (for example demecolcine), a taxane (for example paclitaxel, docetaxel or paclitaxel poliglumex) or another plant alkaloids or natural product (ATC code L01cx, for example trabectedin).

In one embodiment the antineoplastic agent is a cytotoxic antibiotic or related substance selected from the group consisting of an actinomycine (for example dactinomycin), an anthracycline or related substance (for example doxorubicin, daunorubicin, epirubicin, aclarubicin, zorubicin, idarubicin, mitoxantrone, pirarubicin, valrubicin, amrubicin or pixantrone) or another (ATC code L01dc, for example bleomycin, plicamycin, mitomycin or ixabepilone).

In a further embodiment the antineoplastic agent is another antineoplastic agent selected from the group consisting of a platinum compound (for example cisplatin, carboplatin, oxaliplatin, satraplatin or polyplatillen) a methylhydrazine (for example procarbazine) a monoclonal antibody (for example edrecolomab, rituximab, trastuzumab, alemtuzumab, gemtuzumab, cetuximab, bevacizumab, panitumumab, catumaxomab or ofatumumab) a sensitizer used in photodynamic/radiation therapy (for example porfimer sodium, methyl aminolevulinate, aminolevulinic acid, temoporfin or efaproxiral) or a protein kinase inhibitor (for example imatinib, gefitinib, erlotinib, sunitinib, sorafenib, dasatinib, lapatinib, nilotinib, temsirolimus, everolimus, pazopanib, vandetanib, afatinib, masitinib or toceranib).

In a still further embodiment the antineoplastic agent is another neoplastic agent selected from the group consisting of amsacrine, asparaginase, altretamine, hydroxycarbamide, lonidamine, pentostatin, miltefosine, masoprocol, estramustine, tretinoin, mitoguazone, topotecan, tiazofurine, irinotecan, alitretinoin, mitotane, pegaspargase, bexarotene, arsenic trioxide, denileukin diftitox, bortezomib, celecoxib, anagrelide, oblimersen, sitimagene ceradenovec, vorinostat, romidepsin, omacetaxine mepesuccinate or eribulin.

The second aspect of the present invention provides an array for determining the presence of breast cancer in an individual comprising one or more binding agent wherein the one or more binding agent comprises or consists of the one or more binding agents as defined in the first embodiment of the invention.

In an alternative or additional embodiment the one or more binding agents is capable of binding to all of the proteins defined in Table A or Table B.

The first binding agents of the array may be immobilised.

Arrays per se are well known in the art. Typically they are formed of a linear or two-dimensional structure having spaced apart (i.e. discrete) regions (“spots”), each having a finite area, formed on the surface of a solid support. An array can also be a bead structure where each bead can be identified by a molecular code or colour code or identified in a continuous flow. Analysis can also be performed sequentially where the sample is passed over a series of spots each adsorbing the class of molecules from the solution. The solid support is typically glass or a polymer, the most commonly used polymers being cellulose, polyacrylamide, nylon, polystyrene, polyvinyl chloride or polypropylene. The solid supports may be in the form of tubes, beads, discs, silicon chips, microplates, polyvinylidene difluoride (PVDF) membrane, nitrocellulose membrane, nylon membrane, other porous membrane, non-porous membrane (e.g. plastic, polymer, perspex, silicon, amongst others), a plurality of polymeric pins, or a plurality of microtitre wells, or any other surface suitable for immobilising proteins, polynucleotides and other suitable molecules and/or conducting an immunoassay. The binding processes are well known in the art and generally consist of cross-linking covalently binding or physically adsorbing a protein molecule, polynucleotide or the like to the solid support. Alternatively, affinity coupling of the probes via affinity-tags or similar constructs may be employed. By using well-known techniques, such as contact or non-contact printing, masking or photolithography, the location of each spot can be defined. For reviews see Jenkins, R. E., Pennington, S. R. (2001, Proteomics, 2, 13-29) and Lal et al (2002, Drug Discov Today 15; 7 (18 Suppl):S143-9).

Typically the array is a microarray. By “microarray” we include the meaning of an array of regions having a density of discrete regions of at least about 100/cm², and preferably at least about 1000/cm². The regions in a microarray have typical dimensions, e.g. diameter, in the range of between about 10-250 μm, and are separated from other regions in the array by about the same distance. The array may alternatively be a macroarray or a nanoarray.

Once suitable binding molecules (discussed above) have been identified and isolated, the skilled person can manufacture an array using methods well known in the art of molecular biology; see Examples below.

The third aspect of the present invention provides the use of one or more biomarkers selected from the group defined in Table A or Table B as a biomarker for determining the presence of breast cancer in an individual.

In an alternative or additional embodiment all of the proteins defined in Table A or Table B are used as a diagnostic marker for determining the presence of breast cancer in an individual.

The fourth aspect of the present invention provides the use of one or more binding moiety as defined in the first aspect of the invention for determining the presence of breast cancer in an individual.

In an alternative or additional embodiment biomarkers for all of the proteins defined in Table A or Table B are used.

The fifth aspect of the invention provides a kit for determining the presence of breast cancer comprising:
A) one or more binding agent as defined in any one of Claims 59-74 and 81-95 or an array according to Claims 75-79 or Claim 103-104;

B) instructions for performing the method as defined in any one of Claims 1-102.

The sixth aspect of the invention provides a method of treating breast cancer in an individual comprising the steps of:
(a) diagnosing breast cancer according to the method the first aspect of the invention; and
(b) providing the individual with breast cancer therapy.

As noted above, in the event that the individual is not diagnosed with breast cancer, they may be subjected to further monitoring for breast cancer (for example, using the method of the present invention).

In an alternative or additional embodiment the breast cancer therapy is selected from the group consisting of surgery, chemotherapy, immunotherapy, chemoimmunotherapy and thermochemotherapy.

Preferred, non-limiting examples which embody certain aspects of the invention will now be described, with reference to the following figures:

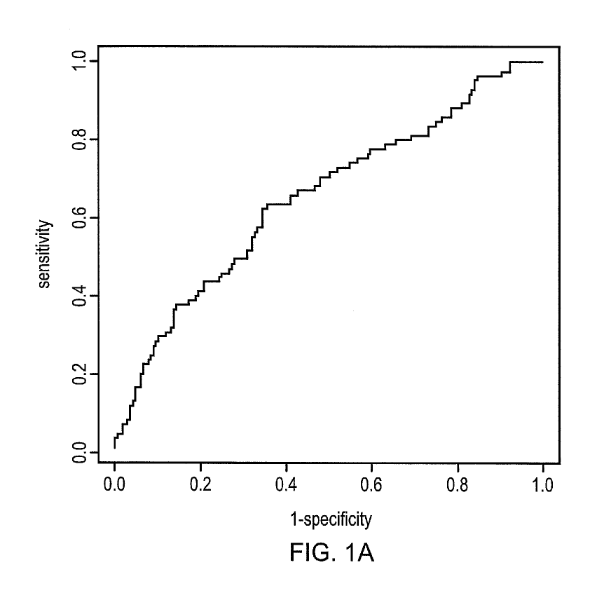

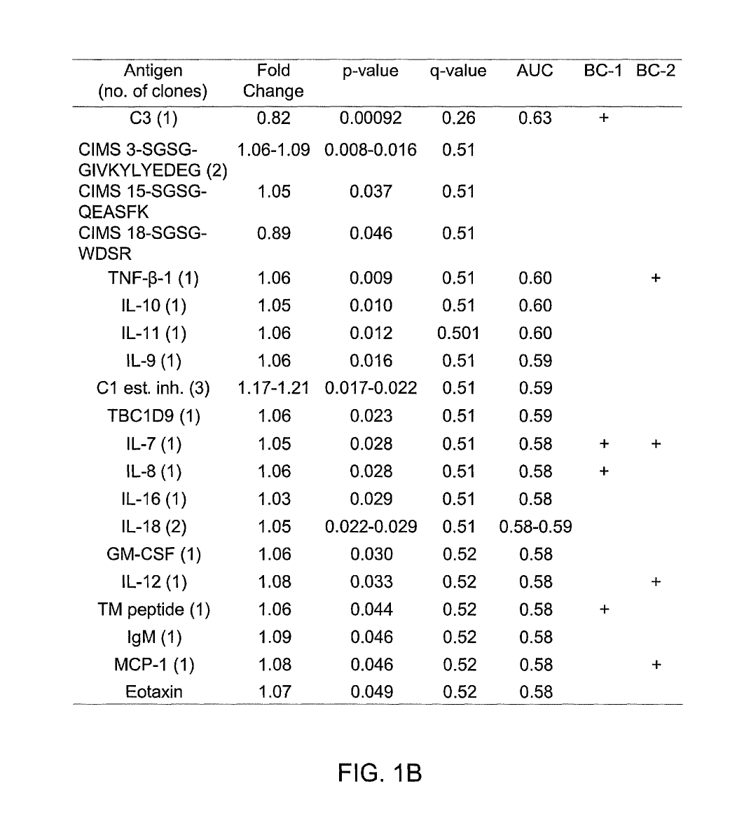

FIG. 1

Classification of early (pre-diagnosed) breast cancer (BC) vs. healthy controls (N). (A) ROC curve for early BC vs. N. (B) Significantly differentially expressed analytes. A fold change >1 represents an up-regulation in BC vs. N, and vice versa. BC-1 represents a 9 serum biomarker signature reflecting BC at time of diagnosis, including patients taking inflammatory drugs and/or hormones, data adopted from (21). BC-2 represents a 8 serum biomarker signature reflecting BC at time of diagnosis, excluding patients taking inflammatory drugs and/or hormones, data adopted from (21).

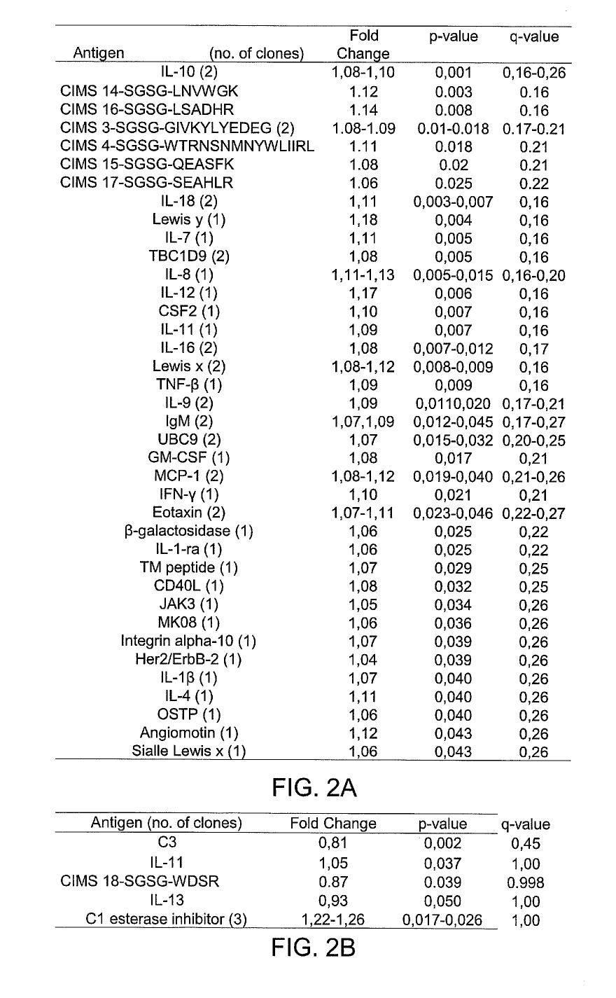

FIG. 2

Immunoprofiling of early (pre-diagnosed) breast cancer (BC) vs. healthy controls (N), where the BC samples were divided into two cohorts based on the time of sample collection prior to diagnosis (0-52 and 52-104 weeks). (A) Significantly differentially expressed analytes for BC (weeks 0-52) vs. N. A fold change >1 represents an up-regulation in BC vs. N, and vice versa. (B) Significantly differentially expressed analytes for BC (weeks 52-104) vs. N.

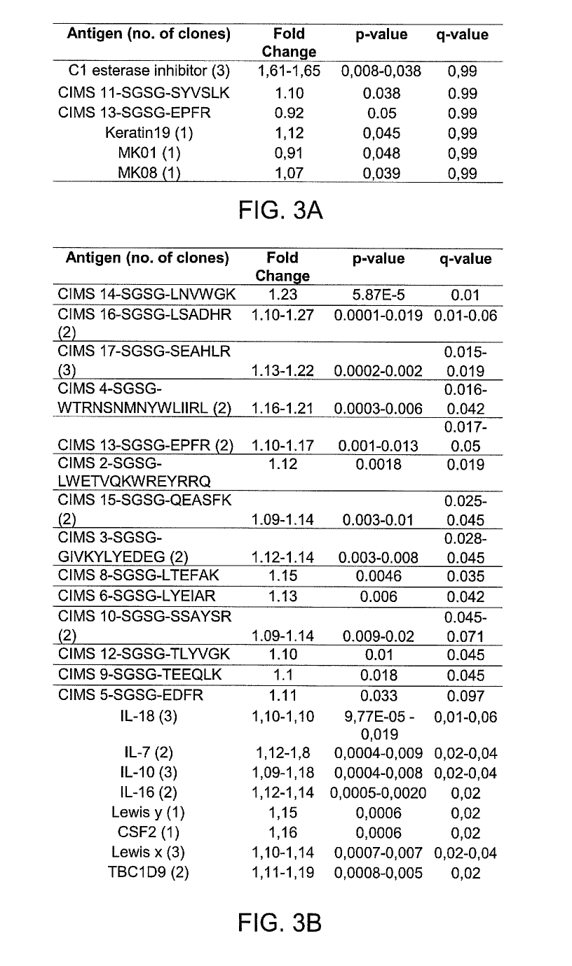

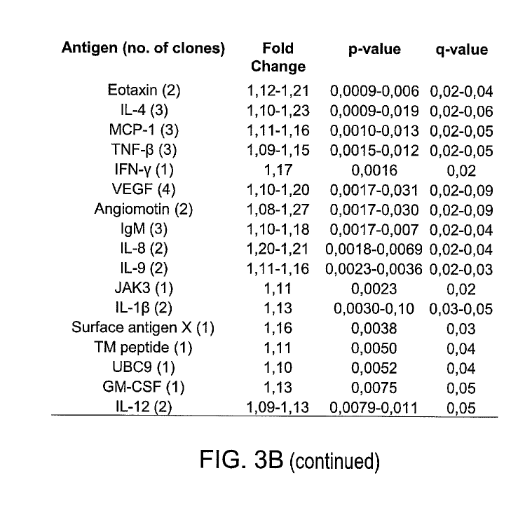

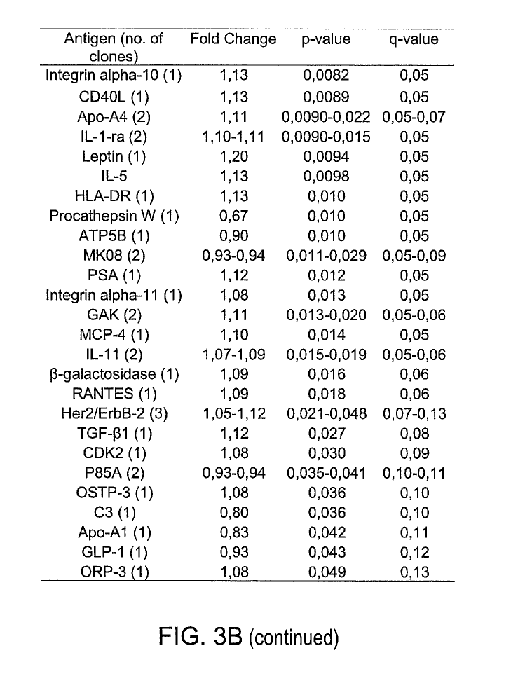

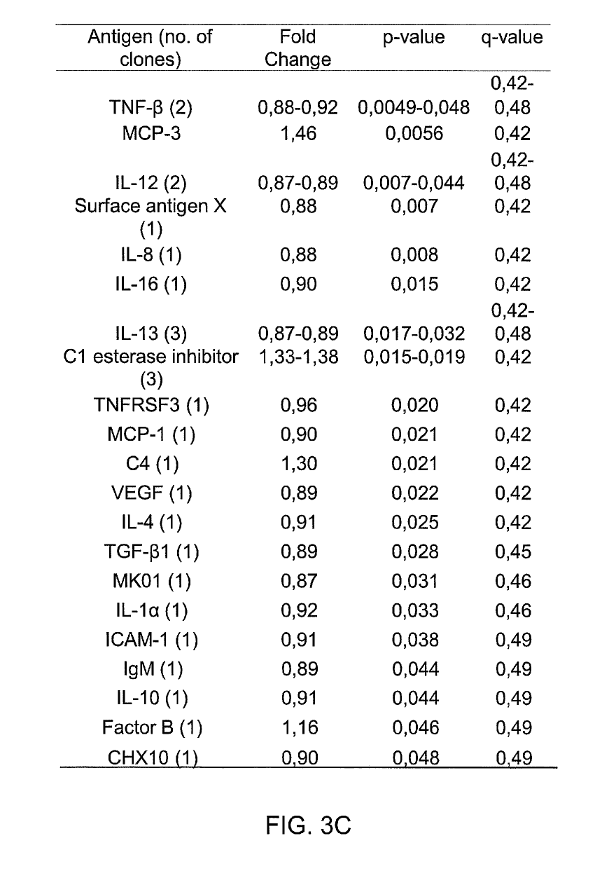

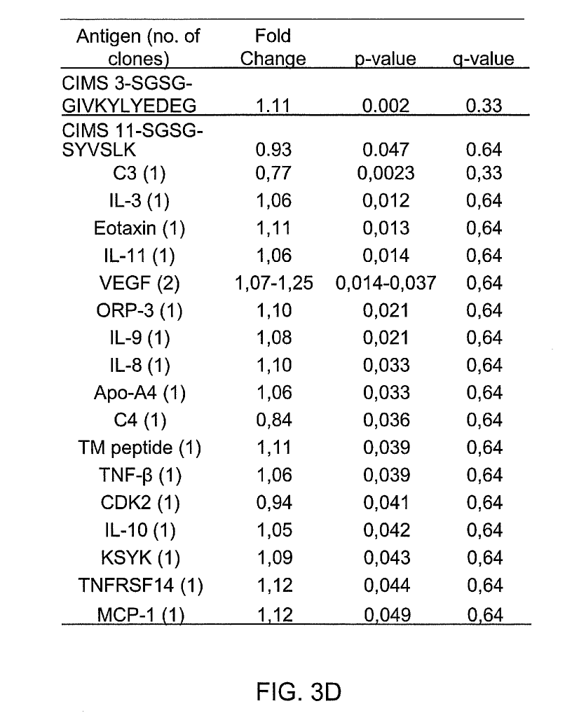

FIG. 3

Immunoprofiling of early (pre-diagnosed) breast cancer (BC) vs. healthy controls (N), where the BC samples were divided into four cohorts based on the time of sample collection prior to diagnosis (0-26, 26-52, 52-78, and 78-104 weeks). (A) Significantly differentially expressed analytes for BC (weeks 0-26) vs. N. A fold change >1 represents an up-regulation in BC vs. N, and vice versa. (B) Significantly differentially expressed analytes for BC (weeks 26-52) vs. N. (C) Significantly differentially expressed analytes for BC (weeks 52-78) vs. N. (D) Significantly differentially expressed analytes for BC (weeks 78-104) vs. N.

FIG. 4

Immunoprofiling of early (pre-diagnosed) breast cancer (BC), where the BC samples were divided into four cohorts based on the time of sample collection prior to diagnosis (0-26, 26-52, 52-78, and 78-104 weeks). (A) Classification of BC (weeks 0-26) vs. BC (weeks 26-52) vs. BC (weeks 52-78) vs. BC (weeks 78-104). The ROC AUC values (LOO cross-validation) are stated. (B) The number of differentially expressed analytes for BC (weeks 0-26) vs. BC (weeks 26-52) vs. BC (weeks 52-78) vs. BC (weeks 78-104). In each comparison, a majority of the de-regulated analytes were either up- or down-regulated, as indicated by the direction of the arrows. (C) The top 15 differentially expressed analytes for BC (weeks 0-26) vs. BC (weeks (26-52). (D) The top 15 differentially expressed analytes for BC (weeks 26-52) vs. BC (weeks (52-78). (E) The top 15 differentially expressed analytes for BC (weeks 52-78) vs. BC (weeks (78-104).

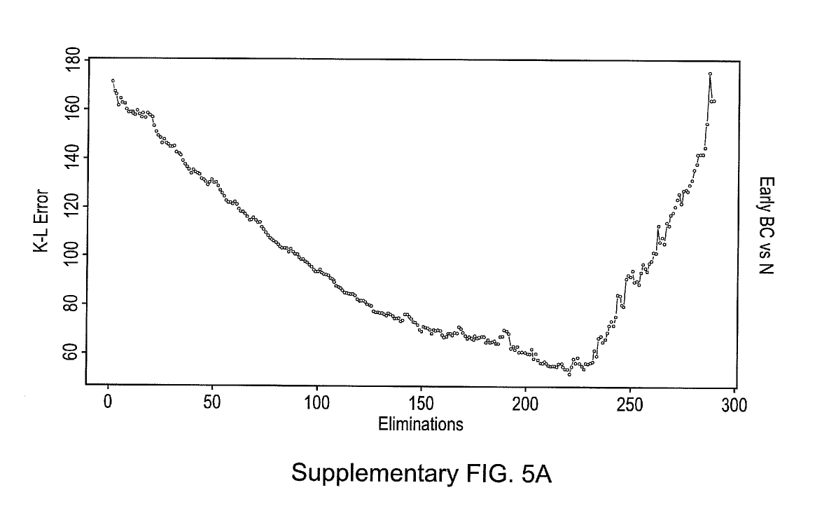

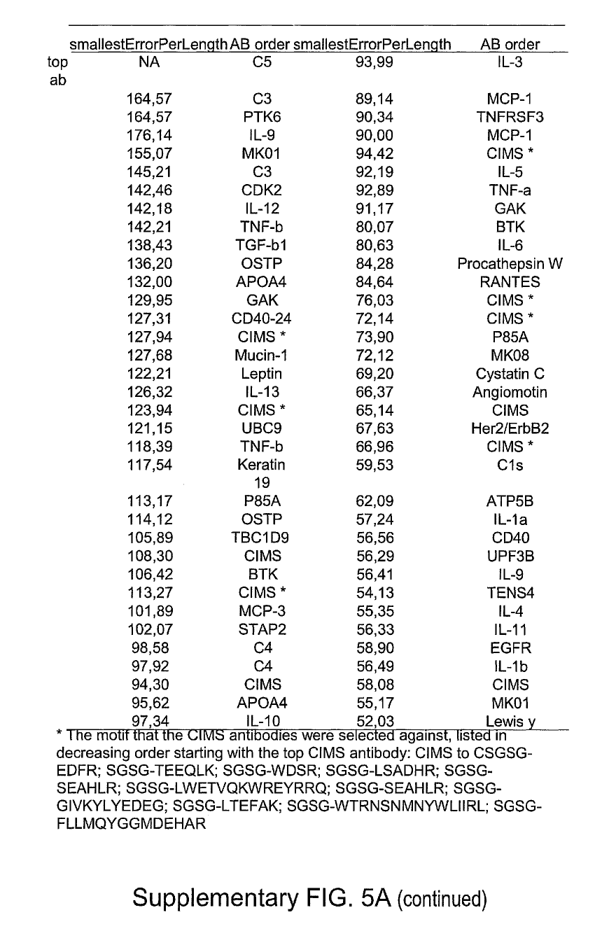

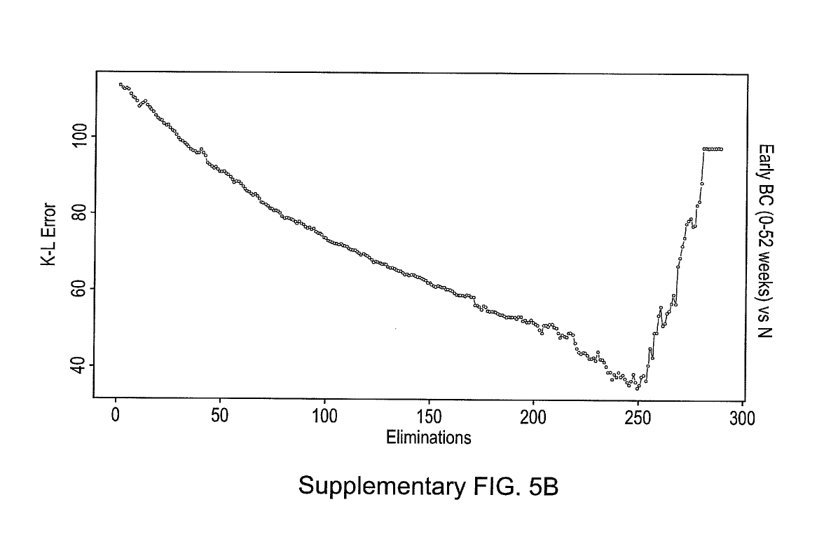

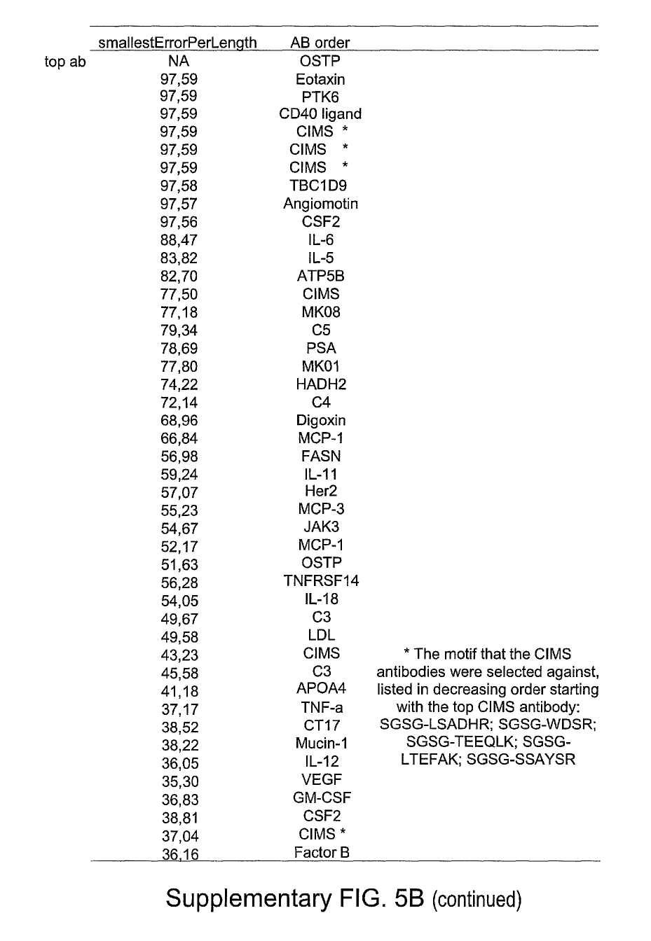

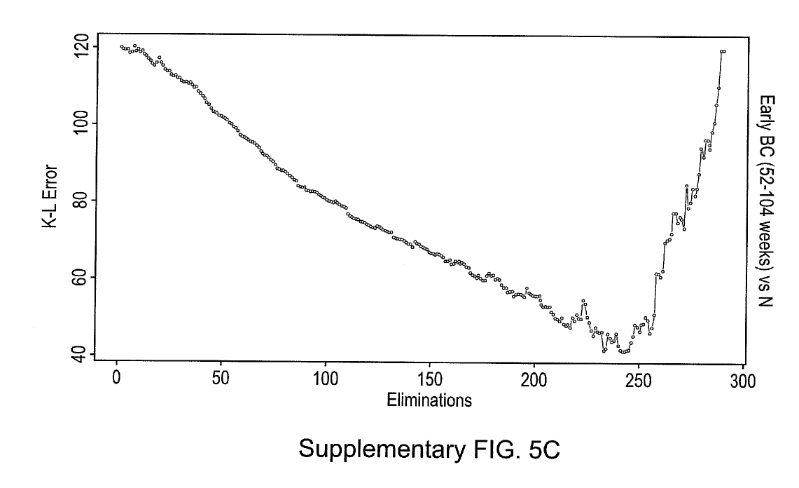

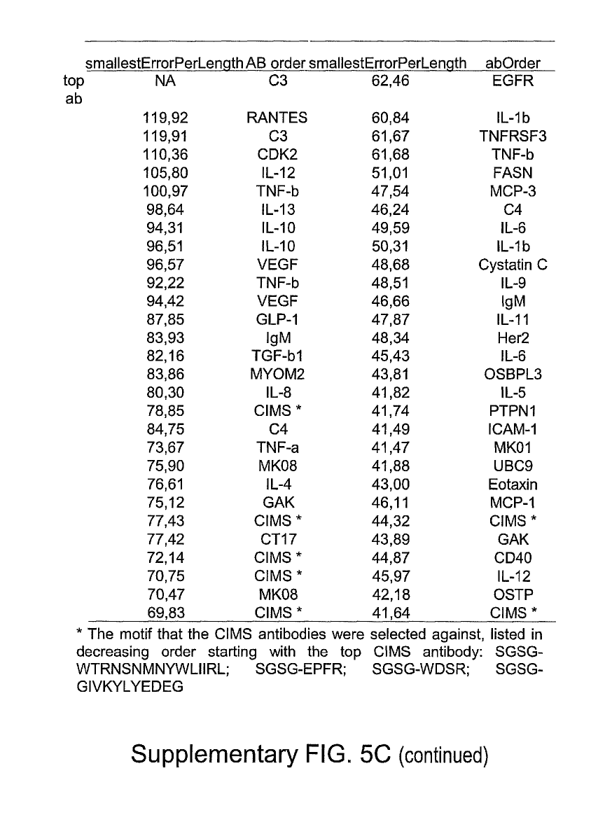

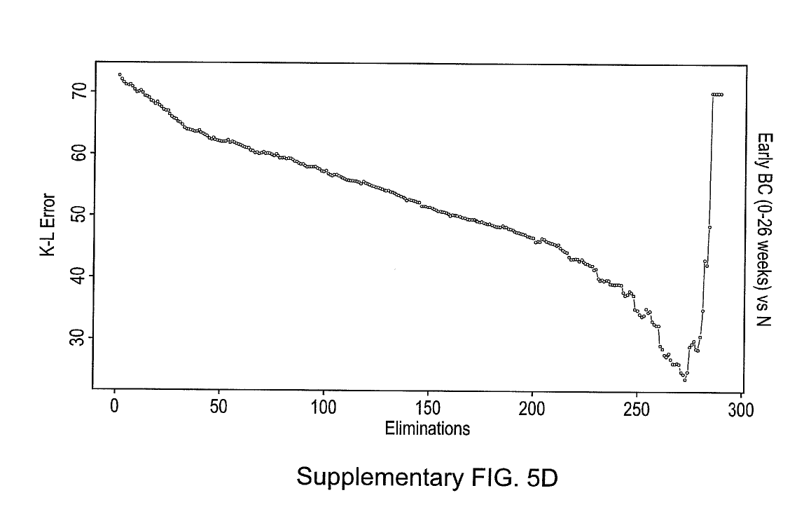

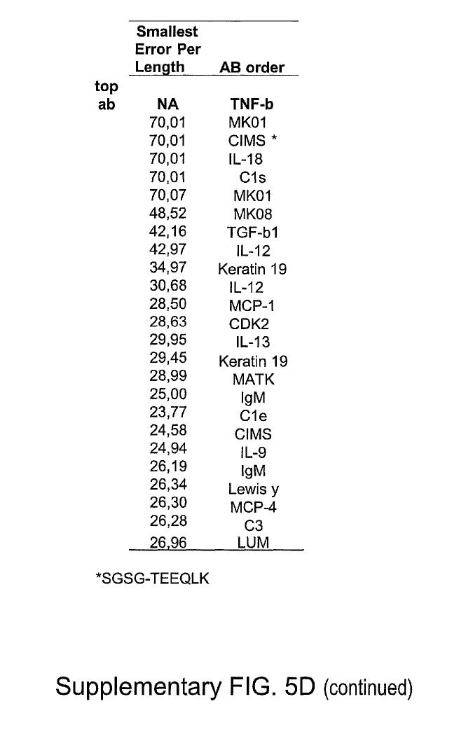

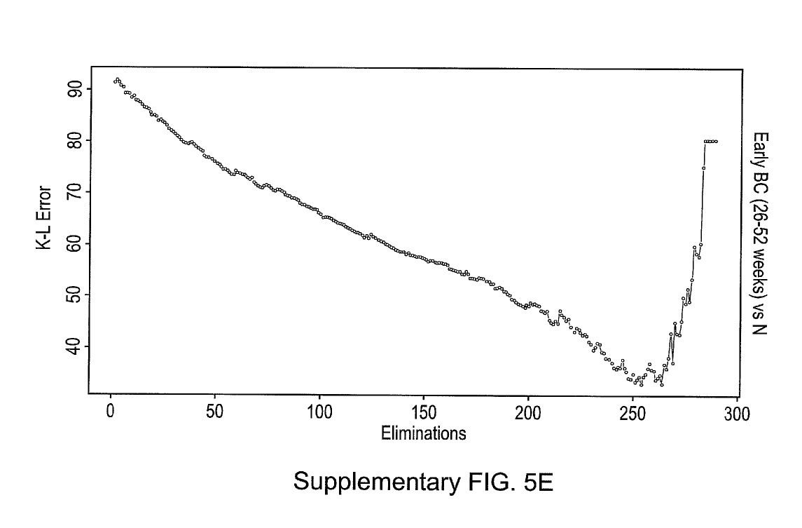

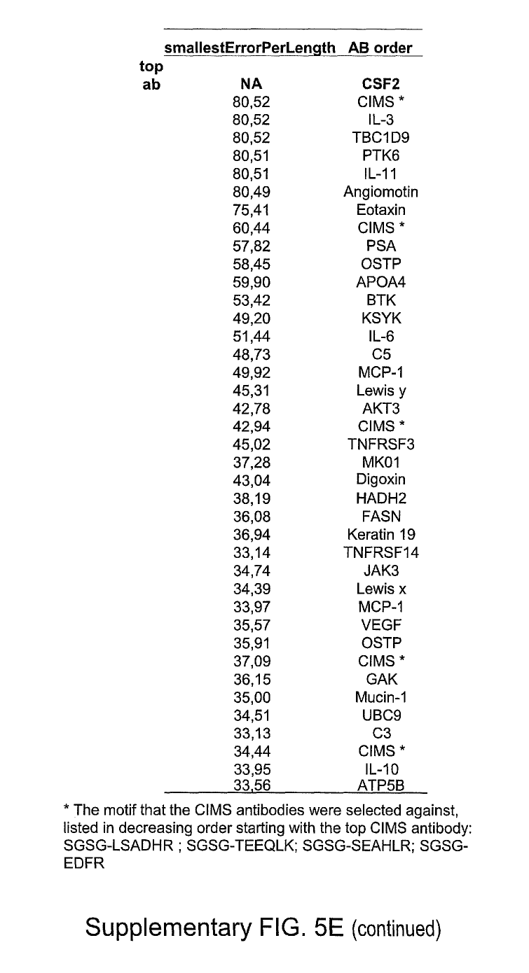

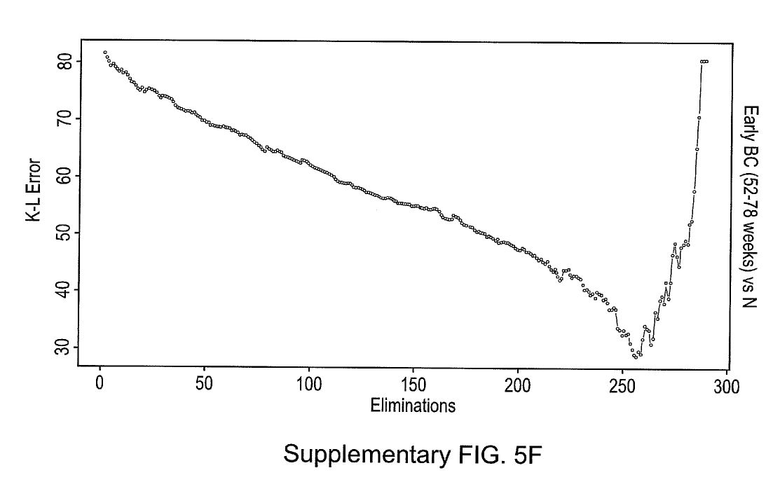

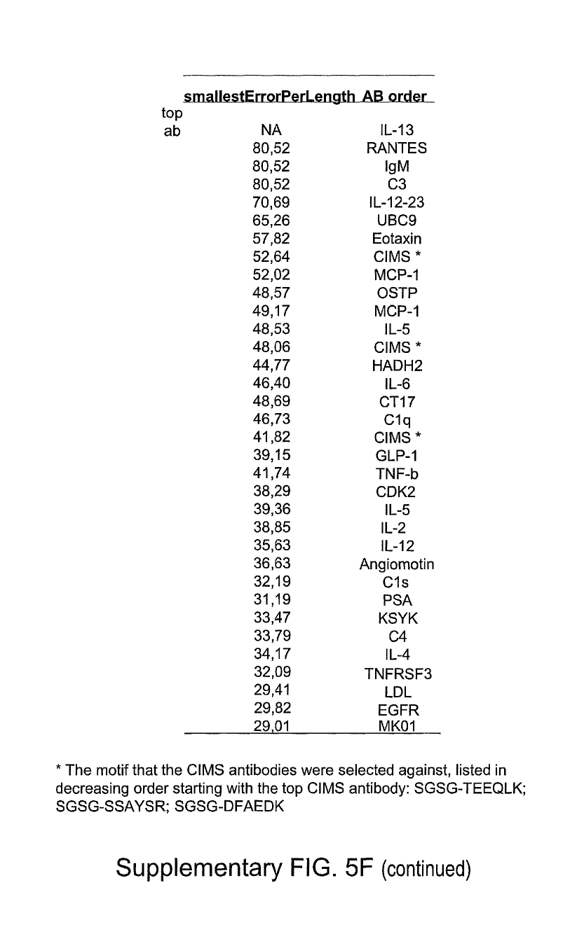

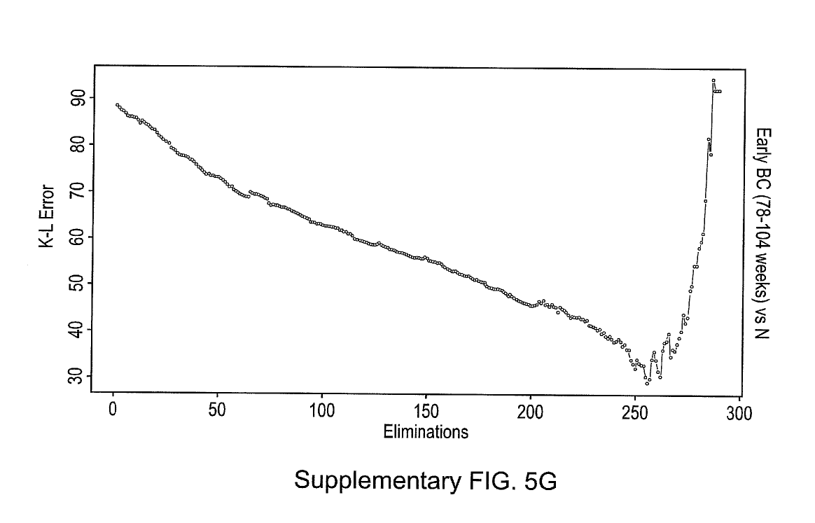

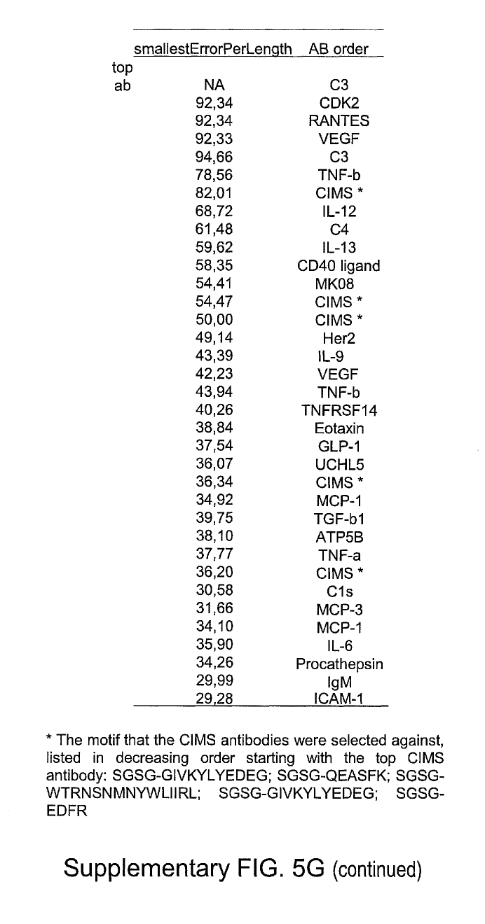

FIG. 5

Immunoprofiling of early (pre-diagnosed) breast cancer (BC), where the BC samples were divided into four cohorts based on the time of sample collection prior to diagnosis (0-26, 26-52, 52-78, and 78-104 weeks). (A) Expression pattern for ten selected key analytes. Significantly differentially up- or down-regulated analytes are indicated by an arrow. (B) Expression pattern for three selected key analytes, in terms of the observed antibody microarray signal intensities. The intensities are normalized and logged.

Supplementary FIG. 1

Correlation between tumor size at time of diagnosis and time (weeks) for sample collection prior to diagnosis.

Supplementary FIG. 2

Mapping of clinical parameters onto the early BC samples, and stratification using PCA analysis. (A) ER positive vs. ER negative. (B) PgR positive vs. PgR negative. (C) Histological grade 1 vs. grade 2 vs. grade 3. (D) Pre-menopausal vs. post-menopausal. (E) BMI group 1 (<18.5) vs. group 2 (18.5-24.9) vs. group 3 (25.0-29.9) vs. group 4 (30.0-34.9) vs. group 5 (35.0-39.9) vs. group 6 (>6). The grouping was based on the guidelines from World Health Organization.

Supplementary FIG. 3

Immunoprofiling of early (pre-diagnosed) breast cancer (BC), where the BC samples were filtered for tumor size 20 mm to be included) and divided into three cohorts based on the time of sample collection prior to diagnosis (0-35, 35-69, and 70-104 weeks). (A) Significantly differentially expressed analytes for BC (weeks 0-35) vs. N. A fold change >1 represents an up-regulation in BC vs. N, and vice versa. (B) Significantly differentially expressed analytes for BC (weeks 70-104) vs. N.

Supplementary FIG. 4

Immunoprofiling of early (pre-diagnosed) breast cancer (BC), where the BC samples were divided into four cohorts based on the time of sample collection prior to diagnosis (0-26, 26-52, 52-78, and 78-104 weeks). (A) Significantly differentially expressed analytes for BC (weeks 0-26) vs. BC (weeks 26-52). A fold change >1 represents an up-regulation in BC vs. N, and vice versa. (B) Significantly differentially expressed analytes for BC (weeks 26-52) vs. BC (weeks 52-78). (C) Significantly differentially expressed analytes for BC (weeks 52-78) vs. BC (weeks 78-104).

Supplementary FIG. 5

The smallest panel of antibodies required to achieve the best classification (minimized error) of the various early BC cohorts vs. N, using a backward elimination strategy was implemented. (A) Early BC (all samples) vs. N. (B) Early BC (0-52 weeks) vs. N. (C) Early BC (52-104 weeks) vs. N. (D) Early BC (0-26 weeks) vs N. (E) Early BC (26-52 weeks) vs. N. (F) Early BC (52-78 weeks) vs. N. (G) Early BC (78-104 weeks) vs. N.

### EXAMPLES

### Introduction

There is a significant need for deciphering disease-associated biomarkers in early breast cancer (BC), which could pave the way for early and improved diagnosis, as well as provide a deeper understanding of the underlying disease biology and processes involved in early BC. In this study, we have for the first time performed immunoprofiling of early human BC by targeting human serum samples collected up to two years before clinical diagnosis. To this end, we utilized the immune system as an early and specific sensor for disease, by profiling predominantly immunoregulatory and cancer-associated analytes using affinity proteomics. The data showed that several disease progression associated serum biomarkers could be delineated in early human BC. The observed serological profiles shed light on biological processes involved in early BC, such as cancer immunoediting, and provide novel opportunities for early BC diagnosis and classification. Taken together, this study demonstrated that a minimally invasive blood sample harbored cancer-specific information, reflecting early human BC and key associated biological processes thereof (at least) up to two years before diagnosis.

Material and Methods

Clinical Samples

The Malmö Diet and Cancer study is a population-based, prospective study in which a total of 17,035 women (born between 1923 and 1950) were enrolled and followed (between 1991 and 1996) (24, 25). After informed consent, serum samples were collected at time of enrollment and stored at −80° until use. A total of 255 patients were selected, including 85 patients clinically diagnosed with breast cancer (BC)<2 years after enrollment (i.e. sample collection), and 170 patients (healthy controls, N) matched with age and weight (body mass index, BMI).

The serum samples were biotinylated, using a previously optimized protocol (18, 19). Briefly, crude samples were diluted 1:45 in PBS, resulting in an approximate protein concentration of 2 mg/mL, and labeled with a 15:1 molar excess of biotin to protein, using 0.6 mM EZ-Link Sulfo-NHS-LC-Biotin (Pierce, Rockford, Ill., USA). Unbound biotin was removed by dialysis against PBS for 72 hours. Labeled samples were aliquoted and stored at −20° C. until further use.

Antibodies

In total, 293 human recombinant single-chain Fv (scFv) antibodies directed against 98 antigens and 31 peptide motifs, denoted CIMS (contex-independent motif specific) antibody clones 1-31 (26), were used as microarray content (Table 2). A majority of the antibodies were selected against immunoregulatory analytes and cancer-associated proteins in order to exploit the immune system as an early and specific sensor for disease (23). The specificity, affinity (normally in the nM range), and on-chip functionality of these phage display derived scFv antibodies (27) (Sall et al, 2014 manuscript in preparation) was ensured by using i) stringent phage-display selection and screening protocols, ii) multiple clones (1-4) per target, and iii) a molecular design, adapted for microarray applications (28). In addition, the specificity of several of the antibodies has previously also been validated using well-characterized, standardized serum samples (with known levels of the targeted analytes), and/or orthogonal methods, such as mass spectrometry (affinity pull-down experiments), ELISA, MesoScaleDiscovery (MSD) assay, cytometric bead assay, and MS, as well as using spiking and blocking experiments (29-37). Notably, the reactivity of some antibodies might be lost since the label (biotin) used to enable detection could block the affinity binding to the antibodies (epitope masking), but we have bypassed this problem, as in this study, by frequently including more than one antibody against the same protein, but directed against different epitopes (28).

The antibodies were produced in 15 mL E. coli cultures and purified from the periplasm in 300 μL, using a MagneHis Protein Purification system (Promega, Madison, Wis., USA) and a KingFisher96 robot (Thermo Fisher Scientific, Waltham, Mass., USA). The elution buffer was exchanged for PBS, using Zeba 96-well desalt spin plates (Pierce). The protein concentration was measured, using NanoDrop (Thermo Scientific, Wilmington, Del., USA) and the purity was checked, using 10% SDS-PAGE (Invitrogen, Carlsbad, Calif., USA).

Antibody Microarrays

The antibody microarray analysis was performed using a previously optimized protocol (17, 38) (Delfani et al, manuscript in preparation). The antibody microarrays were produced on black MaxiSorp slides (Nunc, Roskilde, Denmark) using a non-contact printer (SciFlexarrayer S11, Scienon, Berlin, Germany). Thirteen identical subarrays were printed on each slide, consisting of 33×31 spots, with a spot diameter of 130 μm, and a spot center-to-center distance of 200 μm. Each subarray was divided into three segments, separated by printed rows of labeled BSA. Each antibody was spotted in triplicates, one replicate in each segment. 10 slides were printed each day, resulting in a total of 130 subarrays per day, for three days. The slides were produced over night, and the arrays were subsequently used for array analysis the following day.

Each slide was mounted in a hybridization gasket (Schott, Jena, Germany) and blocked with 1% (w/v) milk, 1% (w/v) Tween-20 in PBS (MTPBS) for one hour. In the meantime, samples were thawed on ice, and diluted 1:10 in MTPBS in Costar non-treated 96-well plates (Corning, N.Y., USA). The slides were washed 4 times with 0.05% (v/v) Tween-20 in PBS (PBST) before 120 μL of the samples were added. Samples were incubated for 2 hours on a rocking table, slides washed 4 times with PBST, incubated with 1 μg/mL Streptavidin-Alexa in PBSMT for 1 hour on a rocking table, and again washed 4 times with PBST. Finally, the slides were dismounted from the hybridization chambers, directly immersed in dH₂O, and dried under a stream of N2. The slides were immediately scanned in a confocal microarray scanner (PerkinElmer Life and Analytical Sciences, Wellesley, Mass., USA) at 10 μm resolution, using 60% PMT gain and 90% laser power. Signal intensities were quantified using the ScanArray Express software version 4.0 (Perkin Elmer Life and Analytical Sciences), and the fixed circle option. Signal intensity values with local background subtraction were used for data analysis.

Microarray Data Pre-Processing

The antibody microarray data was pre-processed using a previously designed strategy (17, 38) (Delfani et al, manuscript in preparation). An average value of three replicate spots, spread out over the array, was used unless any replicate coefficient of variation (CV) exceeded 15% from the mean value, in which case it was dismissed, and the average value of the two remaining replicates was used instead. The average CV of replication was 7%. Applying a cut-off CV of 15%, 88% of the data values were calculated from all three replicate spots, and the remaining 12% from two replicates.

The limit of detection (LOD) was defined as the average blank signal (PBS) plus 2 standard deviations. Any antibodies from which signal intensities were found to be below LOD in >30% of samples were removed, resulting in the removal of four antibody clones against GLP-1R, MCP-4, IL-3, and STAT1. Hence, the subsequent microarray data analysis was performed based on 289 antibodies.

For evaluation of normalization strategies and initial analysis on variance, the data was visualized using principal component analysis (PCA) and hierarchical clustering (QIucore, Lund, Sweden). PCA on log 2 raw data showed some systematic differences between days of analysis, and minor systematic differences between arrays within in the same day of analysis. These differences were effectively neutralized by normalization, which was carried out in two steps. First, the differences between days of analysis were eliminated using a subtract by group mean strategy (39). Briefly, the average intensity for each antibody over all the samples within one day was calculated, and subtracted from the single values, thus zero centering the data. To avoid negative values, the global mean signal of each antibody was then added to each respective data point. In a next step, the array to array differences observed within the same day of analysis were removed by using a semi-global normalization approach reported earlier (17, 38) (Delfani et al, manuscript in preparation), with a minor modification. Thus, a scaling factor was calculated for each subarray, based on the 15% of antibodies with the lowest standard deviation (previously CV) over all samples. This scaling factor was then applied to the data from each sample.

Microarray Data Analysis

The antibody microarray data was analysed using a previously designed strategy (17, 38) (Delfani et al, manuscript in preparation). The BC patients were analysed as one (n=85), two (samples collected <52 (n=34) vs. 52-104 (n=51) weeks prior to diagnosis), or four cohorts (samples collected <26 (n=13) vs. 26-52 (n=21) vs. 52-78 (n=21) vs. 78-104 (n=39) weeks prior to diagnosis). In one set of analysis, the BC patients were first filtered for tumor size 20 mm), before the remaining patients were divided into three cohorts (samples collected <35 (n=16) vs. 35-69 (n=16) vs. 70-104 (n=25) weeks prior to diagnosis). All statistical analysis was based on two-group comparisons. In an attempt to classify BC, and subsets thereof, vs. N (n=170), we used support vector machine (SVM), a supervised learning method in R (40-42) that creates a hyperplane between two pre-defined groups of data. The SVM was trained using leave-one-out (LOO) cross-validation, where one sample is left out while creating the hyperplane, after which the classifier tried to correctly classify the left-out sample. After each iteration, a decision value was calculated based on the distance between the sample and the hyperplane. Based on the decision values, a receiver operating characteristics (ROC) curve was constructed and area under the curve (AUC) value was calculated. No filtration of the data was performed before training the data, i.e. data from all antibodies on the arrays were included in the analysis. ROC AUC values were also calculated for each single antibody using the array signal intensities. Given the expression values for a given antibody, then each sample was classified as either positive or negative by introducing a cut such that e.g. the sample was positive if the signal was larger than this cut. Thus, a specific cut resulted in a sensitivity-specificity pair by comparing with the true sample labels. A ROC curve was then computed by considering all possible cuts for this antibody. Significantly up- or down-regulated analytes (p<0.05) were defined based on relative protein levels and identified using Wilcoxon's signed-rank test. The Benjamini-Hochberg procedure was used for false discovery rate control (q-values) (43). Clinical parameter was mapped onto the BC samples, and visualized/stratified using PCA analysis (QIucore). In order to identify panels of antibodies with the most discriminatory power between two groups, a cross-validated backward elimination strategy was applied, as described previously (21). Briefly, the strategy involved identifying members (antibodies) recognizing orthogonal patterns in the dataset, and removing members which did not contribute to the discriminatory power, in an iterative manner, resulting in a list with a minimal number of members (antibodies) which discriminate the two groups most efficiently.

Results

In order to characterize the serological profile of early breast cancer, we performed immunoprofiling of breast cancer patients (n=85) vs. controls (n=170) for which the serum samples were collected prior 104 weeks) to clinical breast cancer diagnosis. To this end, we performed protein expression profiling of crude, biotinylated serum samples using recombinant antibody microarrays targeting predominantly immunoregulatory and cancer-associated analytes.

Classification of Early BC Vs. N

In an attempt to classify early breast cancer (BC) from healthy controls (N), SVM LOO cross-validation on unfiltered data was performed. The results showed that the classification was moderate, as illustrated by a ROC AUC of 0.65 (FIG. 1A), and that 18 differentially expressed (p<0.05) analytes, targeted by 24 antibodies, were deciphered (FIG. 1B). When viewing the classification power of the differentially expressed analytes one-by-one, single ROC AUC values of 0.58 to 0.63 were observed. A majority of the delineated analytes were found to be up-regulated in BC. It should be noted that key analytes a priori known to play a key role in BC and cancer immunoediting, such as IL-10 and IL-12, were among the differentially expressed analytes. In order to define the smallest panel of antibodies required to achieve the best classification (minimized error) of early BC vs. N, a backward elimination strategy was implemented. The results indicated that a panel of 70 antibodies achieved the optimal classification (minimized error) of early BC vs. N (Supplementary FIG. 5A).

In order to investigate the relevance of the apparent early cancer-associated signature, we compared it to two known serum protein signatures found to be associated with BC at the time of diagnosis (FIG. 1B). These two biomarker panels included a 9 biomarker signature (denoted BC-1) including patients taking inflammatory drugs and/or hormones (21), and an 8 biomarker signature (denoted BC-2) excluding patients taking inflammatory drugs and/or hormones (21). The analysis showed that 4 of 9 (BC-1) (C3, IL-7, IL-8, and TM peptide) and 4 of 8 (BC-2) (TNF-β, IL-7, IL-12, and MCP-1) biomarkers found to be differentially expressed for BC vs. N at time of diagnosis were also detected for early BC vs. N, clearly indicating the relevance.

Refined Classification of Early BC Vs. N

To refine the classification of early BC vs. N, the BC samples were divided into two or four cohorts based on the time of sample collection prior to diagnosis, and SVM LOO cross-validation on unfiltered data was performed. Dividing the samples in two cohorts resulted in ROC AUC values of 0.59 (weeks 0-52) and 0.69 (weeks 52-104), respectively (Table 3). Adopting four BC cohorts, the results indicated that the classification was poor to moderate (ROC AUC of 0.5 to 0.72), but improved the earlier the samples had been collected prior to diagnosis with 78-104 weeks >52-78 weeks <26-52 weeks >0-26 weeks (Table 3).

In order to define the smallest panel of antibodies required to achieve the best classification (minimized error) of the early BC cohorts vs. N, a backward elimination strategy was implemented. The results indicated that a panel of 45 antibodies (0-52 weeks vs. N) and 58 antibodies (52-104 weeks vs. N) achieved the best classification (minimized error) of early BC vs. N when BC was divided into two cohorts, respectively (Supplementary FIGS. 5B and 5C). Furthermore, the data implied a panel of 25 antibodies (0-26 weeks vs. N), 40 antibodies (26-52 weeks vs. N), 34 antibodies (52-78 weeks vs. N), and 35 antibodies (78-104 weeks vs. N) antibodies achieved the best classification (minimized error) of early BC vs. N when BC was divided into four cohorts, respectively (Supplementary FIGS. 5D to 5G).

Since the early BC sample cohorts were defined based on time of sample collection prior to diagnosis, the cohorts were, as could be expected, found to be heterogeneous with respect to tumor size (2-120 mm) (Supplementary FIG. 1). In order to explore the impact of tumor size, which is part of determining the stage of the cancer, on the classification, only patients with tumors 20 mm were selected. The remaining BC samples were then divided into three cohorts due to a smaller sample number, and the early BC vs. N classification was re-run (Table 3). The results implied that the classification was poor to moderate (ROC AUC of <0.5 to 0.66), and improved the earlier the samples had been collected prior to diagnosis with 70-104 weeks >35-69 weeks and 0-35 weeks. Hence, a similar pattern of classification was observed whether 2-120 or 2-20 mm sized tumors were included (Table 3), further indicating the possibility for early classification.

Next, we mapped key clinical parameters, such as oestrogen receptor (ER) status, progesterone receptor (PgR) status, histological grade, pre-/post-menopausal status, and BMI, on the BC samples, and examined whether they could be stratified. Albeit limited by the sample number and that all clinical parameters were not recorded for all patients, the results indicated that neither ER status, PgR status, histological grade, pre-/post-menopausal status, nor BMI could be pin-pointed as confounding factors (Supplementary FIG. 2).

Immunoprofiling of Early BC Vs. N

In order to decipher biological differences between early BC vs. N, their serological immunoprofiles were compared and evaluated in terms of the identity, nature and number of differentially (p<0.05) expressed analytes. When the early BC samples were divided into two cohorts, 34 (p<0.05, q<0.3) (0-52 weeks) (FIG. 2A) and 5 (p<0.05, q<1) (52-104 weeks) (FIG. 2B) differentially expressed analytes were identified, indicating that the largest biological differences were observed for samples collected <52 weeks prior to diagnosis (FIG. 2a). Notably, several key analytes known to be associated with breast cancer, e.g. IL-7, IL-8, IL-18, and MCP-1, and breast cancer immunoediting, e.g. IL-4, IL-10, IL12, and IFN-γ, were deciphered.

This scenario could be even further refined by dividing the early BC sample into four cohorts and re-running the analysis (cfs. Table 3 and FIG. 3). The data showed that the number of differentially expressed analytes peaked in the order of 52 (26-52 weeks) (FIG. 3B) >21 (52-78 weeks) (FIG. 3C) >18 (78-104 weeks) (FIG. 3D) >5 (0-26 weeks) (FIG. 3A). In addition, the pattern of de-regulation (up or down) also differed, with mainly up-regulated analytes in 3 cohorts (0-26, 26-52, and 78-104 weeks) and down-regulated 1 cohort (52-78 weeks). Hence, the data indicated that the largest biological differences occurred for samples collected within 26-52 weeks prior to diagnosis, again involving a priori known key analytes, such as IL-10, IL-18, IL-4, IFN-γ, and IL-12. Taken together, the immunoprofiles were found to differ over time, indicating a significant immunoregulatory and/or cancer-associated process taking place in early breast cancer over time, peaking 26-52 weeks before diagnosis.

In an attempt to examine the influence of the tumor size, which is part of determining the stage of the cancer, on the observed biological differences, we again only included patients with 2-20 mm sized tumors, divided into three cohorts, and re-run the immunoprofiling of early BC vs. N. The results showed that the number of differentially expressed analytes decreased in the order of 33 (70-104 week) >1 (0-35) >0 (36-59 weeks) (Table 3 and Supplementary FIG. 3). A majority of the differentially expressed analytes, such as IL-10, IL-4, IFN-γ, VEGF, and IL1α, were found to be up-regulated in BC vs. N. Further, the panel of de-regulated analytes was similar to that observed for the corresponding analysis involving all tumor samples (2-120 mm in size) (cfs. FIG. 3 and Supplementary FIG. 3). Hence, the data implied that significant immunological processes involving similar analytes occurred in tumors of different sizes (2-20 mm vs. 2-120 mm), but at different timelines (70-104 weeks vs. 26-52 weeks).

Immunoprofiling of Early BC

In order to further unravel the molecular pattern of early BC, we compared the serological immunoprofiles of the four BC sub-cohorts, divided based on the time of sample collection prior to diagnosis. Running SVM LOO cross-validation on unfiltered data showed on poor to moderate classification, as illustrated by ROC AUC values of 0.50 to 0.71 (FIG. 4A). The data showed that the 52-78 weeks and 78-104 weeks cohorts displayed the largest differences with respect to AUC values, i.e., the best classification.

When comparing the biological differences in terms of number of differentially expressed analytes, an intricate pattern of numerous up- and down-regulated analytes was observed (FIG. 4B). Viewing the disease progress, going from week 104 to 0, the serological profile appeared to involve a process of predominantly down-regulation (from weeks 78-104 to 52-78), followed by an up-regulation (from weeks 52-78 to 26-52), and yet another period of down-regulation (from weeks 26-52 to 0-26) when approaching clinical diagnosis. As when these cohorts were compared with healthy controls (FIG. 3), the 26-52 weeks cohorts was found to display the largest differences (FIG. 4B). Hence, the data again indicated a significant immunoregulatory and/or cancer-associated process taking place during the progression of the disease towards clinically diagnosed BC, in particular during weeks 26-52.

The top 15 most differentially expressed analytes are displayed for each comparison in FIGS. 4C to 4E, for complete lists see Supplementary FIG. 4. Among these top analytes, several known breast cancer-associated analytes were again found to be differentially expressed, such as IL-8, IL-10, IL12, IL-18, TNF-β, and VEGF. In order to examine the serological profile reflecting the disease progression in more detail, we focused on ten key cancer immunoediting associated analytes and compared their expression profile over time (FIG. 5). A majority of these analytes were found to follow the same overall expression pattern from week 104 to week 0, involving down-regulation (from weeks 78-104 to 52-78), up-regulation (from weeks 52-78 to 26-52), and finally a down-regulation (from weeks 26-52 to 0-26) before diagnosis (FIG. 5A). The detailed protein expression pattern is shown for IL-10, IL-12, and IFN-γ in FIG. 5B, further indicating the apparent correlation in their overall expression pattern. Taken together, immunoprofiling of early breast cancer was found to reveal numerous disease progression associated serum biomarkers.

### DISCUSSION

Major proteomic efforts have been made to decipher BC-associated biomarkers, but a majority of these have used biological samples collected at or after diagnosis (44, 45). To the best of our knowledge, only a few studies have so far been designed to target serum samples collected prior to diagnosis (1, 2, 46), which could open up novel avenues for decoding serological biomarker panels reflecting early BC. Using a mass spectrometry-based discovery approach, Opstal-van Winden and co-workers have indicated a handful of serum biomarkers to be de-regulated in early BC up to three years before diagnosis, such C3a des-arginine anaphylatoxin and apolipoprotein C-I (1, 2). In comparison, our data pin-pointed C3 to be de-regulated in early BC vs. N up to two years before diagnosis. C3 plays a central role in the complement system and contributes to innate immunity, and is proteolytically cleaved to C3a and C3b upon activation of the complement cascade. Further, the authors concluded the need for additional efforts, in particular studies using other analytical techniques (better suited for profiling crude serum samples) to generate more data on early BC (1, 2). In a follow up study, Opstal-van Winden and co-workers then used a bead-based multiplexed immunoassay to target ten pre-selected markers (e.g. CA 19-9, CEA, CA-125, haptoglobin, and leptin), known to be associated with diagnosed BC, to analyse early BC serum samples (46). While the assay worked satisfactorily, the data showed that early BC vs. controls could not be differentiated based on these analytes, indicating the need of defining early BC markers.

In this study, we have for the first time used recombinant antibody microarrays to perform serum protein expression profiling of early human BC, by targeting crude, i.e. non-fractionated, serum samples collected up to two years before diagnosis. In our focused discovery approach, we harvested the immune system as an early sensor for disease by targeting a large set of predominantly immunoregulatory analytes and cancer-associated markers. Our results showed that several de-regulated analytes could be defined in serum samples collected up to two years prior to diagnosis, clearly indicating the applicability of our approach for deciphering early BC associated serum biomarkers.

The analysis showed that early BC vs. N could be classified with moderate performance, illustrated by ROC AUC values of 0.67 (all BC samples), 0.72 (samples collected 70-104 weeks before diagnosis), and 0.71 (tumors 20 mm, and collected 70-104 weeks before diagnosis). Hence, the data indicated novel opportunities for early detection and diagnosis, up to 70-104 weeks before clinical diagnosis. Reviewing the list of de-regulated analytes, many of the proteins have previously been shown to be associated with diagnosed BC (e.g. C3, IL-7, IL-8, and IL-18) (20, 21, 44, 45, 47) and/or early BC (e.g. IL-10 and IL-12) (4, 5, 7, 10, 11), clearly demonstrating the relevance of our findings. It should, however, be noted that these markers were pin-pointed mainly as single or low-plex markers in the previous studies, and not as part of large multiplexed serum biomarker panels as in our study, in particular for early human BC.

Notably, examining the immunoprofiles of early human BC, and cohorts thereof, in more detail revealed several serum biomarkers that have been described as markers for disease progression by the cancer immunoediting concept in mice and/or humans (4, 5, 7, 9-11). In more detail, a pattern of deregulated key cytokines with both anti-tumor properties (e.g. IL-1α, IL-1β, IL-12, and IFN-γ) and tumor promoting, i.e. immunosuppressive, properties (e.g. IL-10, TGF-β, and VEGF) were observed. To the best of our knowledge, this is the first time such detailed multiplexed immunoprofiles of crude serum samples have been described for early human BC, targeting samples collected up to two years before clinical diagnosis.

Compared to the healthy controls, a pattern of mainly up- (in 3 cohorts) or down-regulated (1 cohort) analytes was observed over time when dividing the early BC samples into four time-dependent cohorts. The largest differences, with respect to the number of differentially expressed proteins, was observed for samples collected 26-52 weeks before diagnosis. Hence, the data implied significant immunoregulatory and/or cancer-associated processes taking place in early breast cancer over time, potentially peaking 26-52 weeks before diagnosis. Considering the nature of key de-regulated analytes (e.g. IL-10, IL-12, and IFN-γ etc), this might be interpreted in terms of cancer immunoediting processes (4, 5, 7, 9-11). However, tumors of different sizes, 2-120 mm, were analysed, which might impact the results, since the size is part of determining the stage of the cancer, in other words, in which phase of cancer immunoediting each individual tumor might be. In accordance, the data also indicated that significant immunological processes involving similar analytes occurred in tumors of different sizes (2-20 mm vs. 2-120 mm), but at different timelines (70-104 weeks vs. 26-52 weeks).

When comparing the four cohorts of early BC samples with each other with respect to the nature and number of differentially expressed analytes, the data indicated, as might be expected (4, 5, 7, 9-11), that different and significant immunoregulatory and/or cancer-associated process took place during the progression of the disease towards clinically diagnosed BC, in particular during weeks 26-52. Again, many analytes known to be involved in the cancer immunoediting process (4, 5, 7, 9-11), including cytokines with both anti-tumor properties (e.g. IL-1α, IL-1β, IL-12, IFN-γ, and TNF-α) and immunosuppressive properties (e.g. IL-10, TGF-β, and VEGF) were found to be de-regulated. Many of these key counteracting analytes, such as IL10, IL-12, and IFN-γ were found to display similar expression patterns over time. Although the profiles revealed large changes occurring over time, the ratio (balance) of IL-10 vs. IL-12 or IFN-γ did not change significantly. The balance of these analytes is essential for estimating in which phase of the cancer immunoediting process a specific tumor is (4, 10).

In the first elimination phase, the balance is displaced towards IL-12 and IFN-γ, promoting tumor immunity (4, 10). In the second equilibrium phase, there is a balance between the tumor promoting and immunosuppressive cytokines, while the balance is displaced towards IL-10 (immunosuppression) in the final escape phase (4, 10). Still, the data indicated the potential of studying the detailed serological profile of early BC samples using affinity proteomics, and highlighted the need for analyzing numerous additional well-characterized early BC samples in order to pre-validate the results and to take the data analysis to the next level of resolution.

Taken together, this study demonstrated that a minimally invasive blood sample harbored disease-specific information, i.e., biomarkers reflecting early human BC and key associated biological processes thereof up to two years before diagnosis. Hence, the observed serological profiles sheds further light on biological processes involved in early BC, such as cancer immunoediting, and provides novel opportunities for early BC diagnosis and classification.

### REFERENCES

1. Opstal-van Winden A W, Krop E J, Karedal M H, Gast M C, Lindh C H, Jeppsson M C, et al. Searching for early breast cancer biomarkers by serum protein profiling of pre-diagnostic serum; a nested case-control study. BMC cancer. 2011; 11:381. PubMed PMID: 21871081. Pubmed Central PMCID: 3189190.
2. Opstal-van Winden A W, Vermeulen R C, Peeters P H, Beijnen J H, van Gils C H. Early diagnostic protein biomarkers for breast cancer: how far have we come? Breast cancer research and treatment. 2012 July; 134(1):1-12. PubMed PMID: 22179926.
3. Hanash S. Disease proteomics. Nature. 2003 Mar. 13; 422(6928):226-32. PubMed PMID: 12634796. eng.
4. Irshad S, Grigoriadis A, Lawler K, Ng T, Tutt A. Profiling the immune stromal interface in breast cancer and its potential for clinical impact. Breast care. 2012 August; 7(4):273-80. PubMed PMID: 23904829. Pubmed Central PMCID: 3515791.
5. Dunn G P, Bruce A T, Ikeda H, Old L J, Schreiber R D. Cancer immunoediting: from immunosurveillance to tumor escape. Nature immunology. 2002 November; 3(11):991-8. PubMed PMID: 12407406.
6. Burnet M. Cancer; a biological approach. I. The processes of control. British medical journal. 1957 Apr. 6; 1(5022):779-86. PubMed PMID: 13404306. Pubmed Central PMCID: 1973174.
7. Dunn G P, Koebel C M, Schreiber R D. Interferons, immunity and cancer immunoediting. Nature reviews Immunology. 2006 November; 6(11):836-48. PubMed PMID: 17063185.
8. Shankaran V, Ikeda H, Bruce A T, White J M, Swanson P E, Old L J, et al. IFNgamma and lymphocytes prevent primary tumour development and shape tumour immunogenicity. Nature. 2001 Apr. 26; 410(6832):1107-11. PubMed PMID: 11323675.
9. Dunn G P, Old L J, Schreiber R D. The immunobiology of cancer immunosurveillance and immunoediting. Immunity. 2004 August; 21(2):137-48. PubMed PMID: 15308095.
10. Mittal D, Gubin M M, Schreiber R D, Smyth M J. New insights into cancer immunoediting and its three component phases—elimination, equilibrium and escape. Current opinion in immunology. 2014 April; 27:16-25. PubMed PMID: 24531241.
11. Schreiber R D, Old L J, Smyth M J. Cancer immunoediting: integrating immunity's roles in cancer suppression and promotion. Science. 2011 Mar. 25; 331(6024):1565-70. PubMed PMID: 21436444.
12. Koebel C M, Vermi W, Swann J B, Zerafa N, Rodig S J, Old L J, et al. Adaptive immunity maintains occult cancer in an equilibrium state. Nature. 2007 Dec. 6; 450(7171):903-7. PubMed PMID: 18026089.
13. Mantovani A, Allavena P, Sica A, Ballwin F. Cancer-related inflammation. Nature. 2008 Jul. 24; 454(7203):436-44. PubMed PMID: 18650914.
14. Kraman M, Bambrough P J, Arnold J N, Roberts E W, Magiera L, Jones J O, et al. Suppression of antitumor immunity by stromal cells expressing fibroblast activation protein-alpha. Science. 2010 Nov. 5; 330(6005):827-30. PubMed PMID: 21051638.
15. Teng M W, Swann J B, Koebel C M, Schreiber R D, Smyth M J. Immune-mediated dormancy: an equilibrium with cancer. Journal of leukocyte biology. 2008 October; 84(4):988-93. PubMed PMID: 18515327.
16. Hanahan D, Weinberg R A. Hallmarks of cancer: the next generation. Cell. 2011 Mar. 4; 144(5):646-74. PubMed PMID: 21376230.
17. Borrebaeck C A, Wingren C. Antibody array generation and use. Methods in molecular biology. 2014; 1131:563-71. PubMed PMID: 24515491.
18. Ingvarsson J, Larsson A, Sjoholm A G, Truedsson L, Jansson B, Borrebaeck C A, et al. Design of recombinant antibody microarrays for serum protein profiling: targeting of complement proteins. Journal of proteome research. 2007 September; 6(9):3527-36. PubMed PMID: 17696517.
19. Wingren C, Ingvarsson J, Dexlin L, Szul D, Borrebaeck C A. Design of recombinant antibody microarrays for complex proteome analysis: choice of sample labeling-tag and solid support. Proteomics. 2007 September; 7(17):3055-65. PubMed PMID: 17787036.
20. Carlsson A, Wingren C, Ingvarsson J, Ellmark P, Baldertorp B, Ferno M, et al. Serum proteome profiling of metastatic breast cancer using recombinant antibody microarrays. European journal of cancer. 2008 February; 44(3):472-80. PubMed PMID: 18171612.
21. Carlsson A, Wingren C, Kristensson M, Rose C, Ferno M, Olsson H, et al. Molecular serum portraits in patients with primary breast cancer predict the development of distant metastases. Proceedings of the National Academy of Sciences of the United States of America. 2011 Aug. 23; 108(34):14252-7. PubMed PMID: 21844363. Pubmed Central PMCID: 3161545.
22. Borrebaeck C A, Wingren C. Design of high-density antibody microarrays for disease proteomics: key technological issues. Journal of proteomics. 2009 Aug. 20; 72(6):928-35. PubMed PMID: 19457338.
23. Borrebaeck C A, Wingren C. Recombinant antibodies for the generation of antibody arrays. Methods in molecular biology. 2011; 785:247-62. PubMed PMID: 21901605.
24. Berglund G, Elmstahl S, Janzon L, Larsson S A. The Malmo Diet and Cancer Study. Design and feasibility. Journal of internal medicine. 1993 January; 233(1):45-51. PubMed PMID: 8429286.
25. Manjer J, Carlsson S, Elmstahl S, Gullberg B, Janzon L, Lindstrom M, et al. The Malmo Diet and Cancer Study: representativity, cancer incidence and mortality in participants and non-participants. European journal of cancer prevention: the official journal of the European Cancer Prevention Organisation. 2001 December; 10(6):489-99. PubMed PMID: 11916347.
26. Olsson N, Wallin S, James P, Borrebaeck C A, Wingren C. Epitope-specificity of recombinant antibodies reveals promiscuous peptide-binding properties. Protein science: a publication of the Protein Society. 2012 December; 21(12):1897-910. PubMed PMID: 23034898. Pubmed Central PMCID: 3575919.
27. Soderlind E, Strandberg L, Jirholt P, Kobayashi N, Alexeiva V, Aberg A M, et al. Recombining germline-derived CDR sequences for creating diverse single-framework antibody libraries. Nature biotechnology. 2000 August; 18(8):852-6. PubMed PMID: 10932154.
28. Borrebaeck C K, Wingren C. Recombinant Antibodies for the Generation of Antibody Arrays. In: Korf U, editor. Protein Microarrays. Methods in Molecular Biology. 785: Humana Press; 2011. p. 247-62.
29. Ingvarsson J, Larsson A, Sjöholm A G, Truedsson L, Jansson B, Borrebaeck C A K, et al. Design of Recombinant Antibody Microarrays for Serum Protein Profiling: Targeting of Complement Proteins. Journal of Proteome Research. 2007 2007/09/01; 6(9):3527-36.
30. Kristensson M, Olsson K, Carlson J, Wullt B, Sturfelt G, Borrebaeck C A K, et al. Design of recombinant antibody microarrays for urinary proteomics. PROTEOMICS—Clinical Applications. 2012; 6 (5-6):291-6.
31. Wingren C, Ingvarsson J, Dexlin L, Szul D, Borrebaeck C A K. Design of recombinant antibody microarrays for complex proteome analysis: Choice of sample labeling-tag and solid support. PROTEOMICS. 2007; 7(17):3055-65.
32. Persson J, Backström M, Johansson H, Jirström K, Hansson G C, Ohlin M. Molecular Evolution of Specific Human Antibody against MUC1 Mucin Results in Improved Recognition of the Antigen on Tumor Cells. Tumor Biology. 2009; 30(4):221-31.
33. Gustaysson E, Ek S, Steen J, Kristensson M, Algenas C, Uhlen M, et al. Surrogate antigens as targets for proteome-wide binder selection. New Biotechnology. 2011; 28(4):302-11.
34. Carlsson A, Wuttge D M, Ingvarsson J, Bengtsson A A, Sturfelt G, Borrebaeck C A K, et al. Serum Protein Profiling of Systemic Lupus Erythematosus and Systemic Sclerosis Using Recombinant Antibody Microarrays. Molecular & Cellular Proteomics. 2011 May 1, 2011; 10 (5).
35. Dexlin-Mellby L, Sandstrom A, Centlow M, Nygren S, Hansson S R, Borrebaeck C A K, et al. Tissue proteome profiling of preeclamptic placenta using recombinant antibody microarrays. PROTEOMICS—Clinical Applications. 2010; 4 (10-11):794-807.
36. Ingvarsson J, Wingren C, Carlsson A, Ellmark P, Wahren B, Engstrom G, et al. Detection of pancreatic cancer using antibody microarray-based serum protein profiling. PROTEOMICS. 2008; 8(11):2211-9.
37. Pauly F, Dexlin-Mellby L, Ek S, Ohlin M, Olsson N, Jirstrom K, et al. Protein Expression Profiling of Formalin-Fixed Paraffin-Embedded Tissue Using Recombinant Antibody Microarrays. J Proteome Res. 2013 Oct. 9. PubMed PMID: 24063262.
38. Carlsson A, Wuttge D M, Ingvarsson J, Bengtsson A A, Sturfelt G, Borrebaeck C A, et al. Serum protein profiling of systemic lupus erythematosus and systemic sclerosis using recombinant antibody microarrays. Molecular & cellular proteomics: MCP. 2011 May; 10 (5):M110 005033. PubMed PMID: 21350050. Pubmed Central PMCID: 3098590.
39. Wu Y W, Wooldridge P J. The impact of centering first-level predictors on individual and contextual effects in multilevel data analysis. Nursing research. 2005 May-June; 54(3):212-6. PubMed PMID: 15897797. Epub 2005/05/18. eng.
40. Chih-chung C, Chih-Jen L. LIBSVM: a library for support vector machines. http/::wwwcsientuedutw/cjlin/libsvm. 2007.
41. Cristianini N, Shawe-Taylor J. An introduction to support vector machines and other kernel-based learning methods. Cambridge Univeristy Press. 2000.
42. D'Cruz D P, Khamashta M A, Hughes G R. Systemic lupus erythematosus. Lancet. 2007 Feb. 17; 369(9561):587-96. PubMed PMID: 17307106. eng.
43. Benjamini Y, Hochberg Y. Controlling the False Discovery Rate: A Practical and Powerful Approach to Multiple Testing. Journal of the Royal Statistical Society Series B (Methodological). 1995; 57(1):289-300.
44. Chung L, Baxter R C. Breast cancer biomarkers: proteomic discovery and translation to clinically relevant assays. Expert review of proteomics. 2012 December; 9(6):599-614. PubMed PMID: 23256671.
45. Ross J S, Symmans W F, Pusztai L, Hortobagyi G N. Breast cancer biomarkers. Advances in clinical chemistry. 2005; 40:99-125. PubMed PMID: 16355921.
46. Opstal-van Winden A W, Rodenburg W, Pennings J L, van Oostrom C T, Beijnen J H, Peeters P H, et al. A bead-based multiplexed immunoassay to evaluate breast cancer biomarkers for early detection in pre-diagnostic serum. International journal of molecular sciences. 2012; 13(10):13587-604. PubMed PMID: 23202969. Pubmed Central PMCID: 3497343.
47. Nicolini A, Carpi A, Rossi G. Cytokines in breast cancer. Cytokine & growth factor reviews. 2006 October; 17(5):325-37. PubMed PMID: 16931107.

### TABLES

TABLE A

| | |
|---|---|
| # | Biomarker |

Table A(i) - Core biomarkers

| | |
|---|---|
| 1. | CIMS - SGSG-EDFR |
| 2. | CIMS - SGSG-TEEQLK |
| 3. | CIMS - SGSG-LSADHR |

Table A(ii) - Preferred biomarkers

| | |
|---|---|
| 4. | AKT3 |
| 5. | Angiomotin |
| 6. | Apo-A1 |
| 7. | C1s |
| 8. | C1q |
| 9. | CDK2 |
| 10. | CIMS - SGSG-DFAEDK |
| 11. | CIMS - SGSG-EPFR |
| 12. | CIMS - |
| | SGSG-FLLMQYGGMDEHAR |
| 13. | CIMS - SGSG-GIVKYLYEDEG |
| 14. | CIMS - SGSG-LNVWGK |
| 15. | CIMS - SGSG-LTEFAK |
| 16. | CIMS - |
| | SGSG-LWETVQKWREYRRQ |
| 17. | CIMS - SGSG-LYEIAR |
| 18. | CIMS - SGSG-QEASFK |
| 19. | CIMS - SGSG-SEAHLR |
| 20. | CIMS - SGSG-SSAYSR |
| 21. | CIMS - SGSG-SYVSLK |
| 22. | CIMS - SGSG-TLYVGK |
| 23. | CIMS - SGSG-WDSR |
| 24. | CIMS - |
| | SGSG-WTRNSNMNYWLIIRL |
| 25. | CSF2 |
| 26. | CT17 |
| 27. | Cystatine C |
| 28. | Digoxin |
| 29. | EGFR |
| 30. | FASN |
| 31. | GAK |
| 32. | GM-CSF |
| 33. | HADH2 |
| 34. | Her2/ErbB-2 |
| 35. | HLA-DR |
| 36. | ICAM-1 |
| 37. | IgM |
| 38. | IL-11 |
| 39. | IL-2 |
| 40. | Integrin alpha-11 |
| 41. | JAK3 |
| 42. | Keratin19 |
| 43. | KSYK |
| 44. | Leptin |
| 45. | Lewis y |
| 46. | LUM |
| 47. | MATK |
| 48. | MK01 |
| 49. | MK08 |
| 50. | Mucin-1 |
| 51. | ORP-3 |
| 52. | Osteopontin |
| 53. | P85A |
| 54. | Procathepsin W |
| 55. | Properdine |
| 56. | PSA |
| 57. | PTK6 |
| 58. | PTPN1 |
| 59. | RPS6KA2 |
| 60. | STAP2 |
| 61. | Surface antigen X |
| 62. | TENS4 |
| 63. | TNF-a |
| 64. | TNFRSF14 |
| 65. | TNFRSF3 |
| 66. | UBC9 |
| 67. | UBE2C |
| 68. | UCHL5 |

Table A(iii) - Optional biomarkers

| | |
|---|---|
| 69. | Apo-A4 |
| 70. | ATP5B |
| 71. | BTK |
| 72. | C1 esterase inhibitor |
| 73. | C3 |
| 74. | [symbol] |
| 75. | [symbol] |
| 76. | CD40 |
| 77. | CD40L |
| 78. | CHX10 |
| 79. | Eotaxin |
| 80. | Factor B |
| 81. | GLP-1 |
| 82. | IFN-γ |
| 83. | IL-10 |
| 84. | IL-12 |
| 85. | IL-13 |
| 86. | IL-16 |
| 87. | IL-18 |
| 88. | IL-1a |
| 89. | IL-1b |
| 90. | IL-1-ra |
| 91. | [symbol] |
| 92. | IL-4 |
| 93. | [symbol] |
| 94. | IL-6 |
| 95. | [symbol] |
| 96. | IL-8 |
| 97. | IL-9 |
| 98. | Integrin alpha-10 |
| 99. | LDL |
| 100. | Lewis x |
| 101. | MCP-1 |
| 102. | MCP-3 |
| 103. | MCP-4 |
| 104. | MYOM2 |
| 105. | OSBPL3 |
| 106. | RANTES |
| 107. | Sialle Lewis x |
| 108. | TBC1D9 |
| 109. | TGF-β1 |
| 110. | TM peptide |
| 111. | TNF-b |
| 112. | UPF3B |
| 113. | VEGF |
| 114. | β-galactosidase |

TABLE B

| | | Sub-table (Related (figure(s)) | | | | | | | | | | | |
|---|---|---|---|---|---|---|---|---|---|---|---|---|---|
| | | i | ii | iii | iv | v | vi | vii | viii | | | | |
| | | (1B, | (2A, | (2B, | (3A, | (3B, | (3C, | (3D, | (S3A, | ix | x | xi | xii |
| # | Biomarker | S5A) | S5B) | S5C) | S5B) | S5D) | S5E) | S5F) | S5G) | (S3B) | (S4A) | (S4B) | (S4C) |
| 1. | AKT3 | | | | | d/u | | | | | | | |
| 2. | Angiomotin | d/u | u | | | u | d/u | | | | d | u | d |
| 3. | Apo-A1 | | | | | d | | | | | d/u | u | |
| 4. | Apo-A4 | d/u | d/u | | | u | | u | | | | d/u | |
| 5. | ATP5B | d/u | d/u | | | d | | d/u | | d | u | d | |
| 6. | BTK | d/u | | | | d/u | | | | u | | u | d |
| 7. | C1 esterase inhibitor | u | | u | u | | u | | u | u | d/u | u | d |
| 8. | C1q | | | | | | d/u | | | | | | |
| 9. | C1s | d/u | | | d/u | | d/u | d/u | | | d | | |
| 10. | C3 | d | d/u | d | | d | d/u | d | | d | | d | u |
| 11. | C4 | d/u | d/u | d/u | | | d | d | | | d | | |
| 12. | C5 | d/u | d/u | | | d/u | | | | d | | | |
| 13. | CD40 | d/u | | d/u | | | | | | | | u | d |
| 14. | CD40L | | u | | | u | | d/u | | | | | |
| 15. | CDK2 | d/u | | d/u | d/u | u | d/u | d | | d/u | | u | |
| 16. | CHX10 | | | | | | d | | | | | u | d |
| 17. | CIMS - | d/u | | | | | | | | | d | | |
| | SGSG-FLLMQYGGMDEHAR | | | | | | | | | | | | |
| 18. | CIMS - | d/u | u | | | u | | u | | u | | u | d |
| | SGSG-GIVKYLYEDEG | | | | | | | | | | | | |
| 19. | CIMS - | d/u | | | | u | | | | u | d | u | |
| | SGSG- | | | | | | | | | | | | |
| | LWETVQKWREYRRQ | | | | | | | | | | | | |
| 20. | CIMS - | d/u | u | d/u | | u | | | | u | d | u | |
| | SGSG-WTRNSNMNYWLIIRL | | | | | | | | | | | | |
| 21. | CIMS - SGSG-DFAEDK | d/u | u | d d/u | u/d | u | | d | | d/u | d/u | u | d |
| 22. | CIMS - SGSG-EDFR | d/u | | | | u | | | | | d | u | |
| 23. | CIMS - SGSG-EPFR | | | | d | u | | | | | d | u | |
| 24. | CIMS - SGSG-LNVWGK | | u | | | u | | | | | d | u | |
| 25. | CIMS - SGSG-LSADHR | d/u | u | | | u | | | | | d | u | d |
| 26. | CIMS - SGSG-LTEFAK | d/u | | | | u | | | | | d | u | d |
| 27. | CIMS - SGSG-LYEIAR | | | | | u | | | | | | u | |
| 28. | CIMS - SGSG-QEASFK | u | u | | | u | | | | | d | u | |
| 29. | CIMS - SGSG-SEAHLR | d/u | u | | | u | | | | u | d | u | d |
| 30. | CIMS - SGSG-SSAYSR | | | | | u | | | | | d | u | |
| 31. | CIMS - SGSG-SYVSLK | | | | u | | | d | | d | u | | |
| 32. | CIMS - SGSG-TEEQLK | d/u | | | | u | | | | | | u | d |
| 33. | CIMS - SGSG-TLYVGK | | | | | u | | | | | d | u | |
| 34. | CIMS - SGSG-WDSR | d/u | | d | | | | | | d | | | |
| 35. | CSF2 | | u | | | u | | | | | | u | |
| 36. | CT17 | | d/u | d/u | | | d/u | | | | | | |
| 37. | Cystatine C | d/u | | d/u | | | | | | | | | d |
| 38. | Digoxin | | d/u | | | d/u | | | | | | u | d |
| 39. | EGFR | d/u | | d/u | | | d/u | | | | | u | |
| 40. | Eotaxin | u | u | d/u | | u | d/u | u | | u | d | u | d |
| 41. | Factor B | | d/u | | | | d | | | | | d | u |
| 42. | FASN | | d/u | d/u | | d/u | | | | | u | | |
| 43. | GAK | d/u | | d/u | | u | | | | | | u | |
| 44. | GLP-1 | | | d/u | | d | d/u | d/u | | | | d | |
| 45. | GM-CSF | u | u | | | u | | | | | | u | d |
| 46. | HADH2 | | d/u | | | d/u | d/u | | | | u | d | |
| 47. | Her2/ErbB-2 | d/u | u | d/u | | u | | d/u | | u | | u | |
| 48. | HLA-DR | | | | | u | | | | | d | u | |
| 49. | ICAM-1 | | | d/u | | | d | d/u | | | d | u | d |
| 50. | IFN-γ | | u | | | u | | | | u | | u | |
| 51. | IgM | u | u | d/u | d/u | u | d | d/u | | u | d | u | d |
| 52. | IL-10 | u | u | d/u | | u | d | u | | d/u | d | u | d |
| 53. | IL-11 | u | u | u | | u | | u | | | | u | |
| 54. | IL-12 | u | u | d/u | d/u | u | d | d/u | | | d | u | d |
| 55. | IL-13 | d/u | | d | d/u | | d | d/u | | | | u | |
| 56. | IL-16 | u | u | | | u | d | | | u | d | u | d |
| 57. | IL-18 | u | u | | d/u | u | | | | u | d | u | d |
| 58. | IL-1a | d/u | | | | | d | | | | d | u | d |
| 59. | IL-1b | d/u | u | d/u | | u | | | | | d | u | |
| 60. | IL-1-ra | | u | | | u | | | | | d | u | |
| 61. | IL-2 | | | | | | d/u | | | | | u | |
| 62. | IL-3 | d/u | | | | d/u | | u | | | | | d |
| 63. | IL-4 | d/u | u | d/u | | u | d | | | u | d | u | d |
| 64. | IL-5 | d/u | d/u | d/u | | u | d/u | | | | d | | |
| 65. | IL-6 | d/u | d/u | d/u | | d/u | d/u | d/u | | | d | u | d |
| 66. | IL-7 | u | u | | | u | | | | u | d | u | |
| 67. | IL-8 | u | u | d/u | | u | d | u | | u | d | u | d |
| 68. | IL-9 | u | u | d/u | d/u | u | | u | | u | d | u | |
| 69. | Integrin alpha-10 | | u | | | u | | | | | | u | |
| 70. | Integrin alpha-11 | | | | | u | | | | | d | u | |
| 71. | JAK3 | | u | | | u | | | | | d | u | |
| 72. | Keratin 19 | d/u | | | u | d/u | | | | | | | |
| 73. | KSYK | | | | | d/u | d/u | u | | u | | | d |
| 74. | LDL | | d/u | | | | d/u | | | d | | | |
| 75. | Leptin | d/u | | | | u | | | | | d | u | |
| 76. | Lewis x | | u | | | u | | | | u | u | u | |
| 77. | Lewis y | d/u | u | | d/u | u | | | | | | | |
| 78. | LUM | | | | d/u | | | | | d | | | |
| 79. | MATK | | | | d/u | | | | | | | | d |
| 80. | MCP-1 | u | u | d/u | d/u | u | d | u | | u | d | u | d |
| 81. | MCP-3 | d/u | d/u | d/u | | | u | d/u | | | | u | u |
| 82. | MCP-4 | | | | d/u | u | | | | u | d | | |
| 83. | MK01 | d/u | d/u | d/u | d | d/u | d | | | u | d | d/u | d |
| 84. | MK08 | d/u | u | d/u | u | d | | d/u | | | u | d | d |
| 85. | Mucin-1 | d/u | d/u | | | d/u | | | | | | u | d |
| 86. | MYOM2 | | | d/u | | | | | | | u | d | d |
| 87. | ORP-3 | | | | | u | | u | | | d | u | d |
| 88. | OSBPL3 | | | d/u | | | | | | | | | |
| 89. | OSTP | d/u | u | d/u | | u | d/u | | | | | u | |
| 90. | P85A | d/u | | | | d | | | | d | u | d | |
| 91. | Procathepsin W | d/u | | | | d | | d/u | | d | d | | |
| 92. | Properdine | | | | | | | | | u | d | | |
| 93. | PSA | | d/u | | | u | d/u | | | | d | u | |
| 94. | PTK6 | d/u | d/u | | | | | | | | u | | |
| 95. | PTPN1 | | | d/u | | | | | | | | | |
| 96. | RANTES | d/u | | d/u | | u | d/u | d/u | | | d | u | |
| 97. | RPS6KA2 | | | | | | | | | | | | d |
| 98. | Sialle Lewis x | | u | | | | | | | u | | u | |
| 99. | STAP2 | d/u | | | | | | | | | u | | |
| 100. | Surface antigen X | | | | | u | d | | | | d | u | d |
| 101. | TBC1D9 | u | u | | | u | | | | | d | u | |
| 102. | TENS4 | d/u | | | | | | | | | | | |
| 103. | TGF-β1 | d/u | | d/u | d/u | u | d | d/u | | | d | u | |
| 104. | TM peptide | u | u | | | u | | u | | | | u | |
| 105. | TNF-a | d/u | d/u | d/u | | | | d/u | | | u | u | d |
| 106. | TNF-b | u | u | d/u | d/u | u | d | u | | u | d | u | d |
| 107. | TNFRSF14 | | d/u | | | | | u | | | | | d |
| 108. | TNFRSF3 | d/u | | d/u | | | d | | | | u | u | d |
| 109. | UBC9 | d/u | u | d/u | | u | d/u | | | | | u | |
| 110. | UBE2C | | | | | | | | | | | u | d |
| 111. | UCHL5 | | | | | | | d/u | | | | | d |
| 112. | UPF3B | d/u | | | | | | | | | | | d |
| 113. | VEGF | | d/u | d/u | | u | d | u | | u | d | u | d |
| 114. | β-galactosidase | | u | | | u | | | | u | | u | |
| Total | 114 | 68 | 60 | 43 | 21 | 78 | 40 | 37 | 1 | 38 | 63 | 82 | 47 |

TABLE 1
Demographic data of the patients included in the study.

| Parameter | Breast Cancer | Controls |
|---|---|---|
| No. of samples | 85 | 170 |
| Age, mean (range) | 56.8 (45-71.3) | 56.4 (45.8-71.8) |
| Collection time before diagnosis | 59.8 (1-103) | — |
| (weeks), mean (range) | | |
| Tumor size (mm), mean (range) | 19.9 (2-120) | — |
| Oestrogen receptor (+/−/n.d.) | 61/6/18 | — |
| Progesterone receptor (+/−/n.d.) | 45/22/18 | — |
| Grade (1/2/3/n.d.) | 13/35/19/18 | — |
| Pre/Post menopausal | 26/59 | 48/122 |
| BMI, mean (range) | 25.4 (18.0-40.2) | 25.4 (16.7-47.1) |

n.d. = not determined

TABLE 2
Antigens targeted on the antibody microarray

| | | No of | |
|---|---|---|---|
| | | antibody | |
| Protein | Full name | clones | Uniprot Entry |
| Angiomotin | Angiomotin | 2 | Q4VCS5 |
| Apo-A1 | Apolipoprotein A1 | 3 | P02647 |
| Apo-A4 | Apolipoprotein A4 | 3 | P06727 |
| ATP-5B | ATP synthase subunit beta, | 3 | P06576 |
| | mitochondrial | | |
| b- | Beta-galactosidase | 1 | P16278 |
| galactosidase | | | |
| BTK | Tyrosine-protein kinase BTK | 4 | Q06187 |
| C1 inhibitor | Plasma protease C1 inhibitor | 4 | P05155 |
| C1q* | Complement C1q | 1 | P02745/6/7 |
| C1s | Complement C1s | 1 | P09871 |
| C3* | Complement C3 | 6 | P01024 |
| C4* | Complement C4 | 4 | P0COL4/5 |
| C5* | Complement C5 | 3 | P01031 |
| CD40 | CD40 protein | 4 | Q6P2H9 |
| CD40L | CD40 ligand | 1 | P29965 |
| CDK-2 | Cyclin-dependent kinase 2 | 2 | P24941 |
| CHX10 | Visual system homeobox 2 | 3 | P58304 |
| CIMS** | Context indepndent peptide | 31 | Peptide motifs - not applicable |
| | motifs (4 tp 6 amino acid | | |
| | residues long) | | |
| CT | Cholera toxin subunit B | 1 | P01556 |
| | (control) | | |
| Cystatin C | Cystatin C | 4 | P01034 |
| Digoxin | Digoxin (control) | 1 | no protein, i.e. not applicable |
| DUSP9 | Dual specificity protein | 1 | Q99956 |
| | phosphatase 9 | | |
| EGFR | Epidermal growth factor | 1 | P00533 |
| | receptor | | |
| Eotaxin | Eotaxin | 3 | P51671 |
| Factor B* | Complement factor B | 4 | P00751 |
| FASN | Fatty acid synthase | 4 | Q6PJJ3 |
| GAK | Cyclin G-associated kinase | 3 | Q5U4P5 |
| GLP-1 | Glucagon-like peptide-1 | 1 | P01275 |
| GLP-1R | Glucagon-like peptide 1 | 1 | P43220 |
| | receptor | | |
| GM-CSF | Granulocyte-macrophage | 6 | P04141 |
| | colony-stimulating factor | | |
| HADH2 | 3-hydroxyacyl-CoA | 4 | Q6IBS9 |
| | dehydrogenase type-2 | | |
| Her2/ErbB-2 | Receptor tyrosine-protein | 4 | P04626 |
| | kinase erbB-2 | | |
| HLA-DR/DP | HLA-DR/DP | 1 | P01903/P01911/P13762/Q30154/P20036/P0440 |
| ICAM-1 | Intercellular adhesion | 1 | P05362 |
| | molecule 1 | | |
| IFN-g | Interferon gamma | 3 | P01579 |
| IgM | Immunoglobulin M | 5 | e.g. P01871 (not complete protein); isotype- |
| | | | specific for IgM on Ramos B cells¹⁾ |
| IL-10* | Interleukin-10 | 3 | P22301 |
| IL-11 | Interleukin-11 | 3 | P20809 |
| IL-12* | Interleukin-12 | 4 | P29459/60 |
| IL-13* | Interleukin-13 | 3 | P35225 |
| IL-16 | Interleukin-16 | 3 | Q14005 |
| IL-18 | Interleukin-18 | 3 | Q14116 |
| IL-1a* | Interleukin-1 alpha | 3 | P01583 |
| IL-1b | Interleukin-1 beta | 3 | P01584 |
| IL-1ra | Interleukin-1 receptor | 3 | P18510 |
| | antagonist protein | | |
| IL-2 | Interleukin-2 | 3 | P60568 |
| IL-3 | Interleukin-3 | 3 | P08700 |
| IL-4* | Interleukin-4 | 4 | P05112 |
| IL-5* | Interleukin-5 | 3 | P05113 |
| IL-6* | Interleukin-6 | 8 | P05231 |
| IL-7 | Interleukin-7 | 2 | P13232 |
| IL-8* | Interleukin-8 | 3 | P10145 |
| IL-9 | Interleukin-9 | 3 | P15248 |
| Integrin a- | Integrin alpha-10 | 1 | O75578 |
| 10 | | | |
| Integrin a- | Integrin alpha-11 | 1 | Q9UKX5 |
| 11 | | | |
| JAK3 | Tyrosine-protein kinase JAK3 | 1 | P52333 |
| Keratin19 | Keratin, type I cytoskeletal 19 | 3 | P08727 |
| KSYK | Tyrosine-protein kinase SYK | 2 | P43405 |
| LDL | Apolipoprotein B-100 | 2 | P04114 |
| Leptin | Leptin | 1 | P41159 |
| Lewis x | Lewis x | 2 | carbohydrate, i.e. not applicable |
| Lewis y | Lewis y | 1 | carbohydrate, i.e. not appliable |
| Lumican | Lumican | 1 | P51884 |
| MAPK1 | Mitogen-activated protein | 4 | P28482 |
| | kinase 1 | | |
| MAPK8 | Mitogen-activated protein | 3 | P45983 |
| | kinase 8 | | |
| MATK | Megakaryocyte-associated | 3 | P42679 |
| | tyrosine-protein kinase | | |
| MCP-1* | C-C motif chemokine 2 | 9 | P13500 |
| MCP-3 | C-C motif chemokine 7 | 3 | P80098 |
| MCP-4 | C-C motif chemokine 13 | 3 | Q99616 |
| MUC-1 | Mucin-1 | 6 | P15941 |
| Myomesin-2 | Myomesin-2 | 2 | P54296 |
| ORP-3 | Oxysterol-binding protein- | 2 | Q9H4L5 |
| | related protein 3 | | |
| Osteopontin | Osteopontin | 3 | P10451 |
| P85A | Phosphatidylinositol 3-kinase | 3 | P27986 |
| | regulatory subunit alpha | | |
| PKB | RAC-gamma serine/threonine- | 2 | Q9Y243 |
| gamma | protein kinase | | |
| Procathepsin W | Procathepsin W | 1 | P56202 |
| Properdin* | Properdin | 1 | P27918 |
| PSA | Prostate-specific antigen | 1 | P07288 |
| PTK-6 | Protein-tyrosine kinase 6 | 1 | Q13882 |
| PTP-1B | Tyrosine-protein phosphatase | 3 | P18031 |
| | non-receptor type 1 | | |
| RANTES | C-C motif chemokine 5 | 3 | P13501 |
| RPS6KA2 | Ribosomal protein S6 kinase | 3 | Q15349 |
| | alpha-2 | | |
| Sialyl Lewis x | Sialyl Lewis x | 1 | carbohydrate, i.e. not applicable |
| Sox11A | Transcription factor SOX-11 | 1 | P35716 |
| STAP2 | Signal-transducing adaptor | 4 | Q9UGK3 |
| | protein 2 | | |
| STAT1 | Signal transducer and activator | 2 | P42224 |
| | of transcription 1-alpha/beta | | |
| Surface Ag X | Surface Ag X | 1 | not applicable |
| TBC1D9 | TBC1 domain family member 9 | 3 | Q6ZT07 |
| TENS4 | Tensin-4 | 1 | Q8IZW8 |
| TGF-b1 | Transforming growth factor | 3 | P01137 |
| | beta-1 | | |
| TM peptide | Transmembrane peptide | 1 | peptide antigen, notapplicable |
| TNF-a | Tumor necrosis factor | 3 | P01375 |
| TNF-b* | Lymphotoxin-alpha | 4 | P01374 |
| TNFRSF14 | Tumor necrosis factor receptor | 2 | Q92956 |
| | superfamily member 14 | | |
| TNFRSF3 | Tumor necrosis factor receptor | 3 | P36941 |
| | superfamily member 3 | | |
| UBC9 | SUMO-conjugating enzyme | 3 | P63279 |
| | UBC9 | | |
| UBE2C | Ubiquitin-conjugating enzyme | 2 | O00762 |
| | E2 C | | |
| UCHL5 | Ubiquitin carboxyl-terminal | 1 | Q9Y5K5 |
| | hydrolase isozyme L5 | | |
| UPF3B | Regulator of nonsense | 2 | Q9BZI7 |
| | transcripts 3B | | |
| VEGF* | Vascular endothelial growth | 4 | P15692 |
| | factor | | |

*Antibody specificity determined by protein arrays, MSD, ELISA, blocking/spiking experiments, and/or mass spectrometry.
**31 CIMS clones selected against 18 motifs. Specification of the clones (clone name/linker sequence/selection motif/no. of clones against the motif); CIMS 1-SGSG-FLLMQYGGMDEHAR (1); CIMS 2-SGSG-LWETVQKWREYRRQ (1); CIMS 3-SGSG-GIVKYLYEDEG (2); CIMS 4-SGSG-WTRNSNMNYWLIIRL (2); CIMS 5-SGSG-EDFR (2); CIMS 6-SGSG-LYEIAR (1); CIMS 7-SGSG-DFAEDK (1); CIMS 8-SGSG-LTEFAK (1); CIMS 9-SGSG-TEEQLK (3); CIMS 10-SGSG-SSAYSR (2); CIMS 11-SGSG-SYVSLK (1); CIMS 12-SGSG-TLYVGK (1); CIMS 13-SGSG-EPFR (2); CIMS 14-SGSG-LNVWGK (1); CIMS 15-SGSG-QEASFK (2); CIMS 16-SGSG-LSADHR (2); CIMS 17-SGSG-SEAHLR (4); CIMS 18-SGSG-WDSR (2).

TABLE 3
Classification of early BC patients vs. N after dividing all BC patients into
two or four cohorts based on time of sample collection prior to clinical
diagnosis. The BC patients were also filtered for tumor size (≦20 mm)
and divided into three cohorts, and re-compared to the controls.

| | | |
|---|---|---|
| BC cohort, weeks | | No. of differentially |
| prior to diagnosis | ROC AUC | expressed analytes |

All BC samples

| | | |
|---|---|---|
| 0-52 | 0.59 | 33 |
| 52-104 | 0.67 | 5 |
| 0-26 | 0.50 | 5 |
| 26-52 | 0.51 | 52 |
| 52-78 | 0.57 | 21 |
| 78-104 | 0.72 | 18 |

BC samples with tumor ≦20 mm

| | | |
|---|---|---|
| 0-35 | 0.44 | 1 |
| 35-69 | 0.38 | 0 |
| 70-104 | 0.66 | 33 |

1. A method for diagnosing breast cancer comprising or consisting of the steps of:
a) providing a sample to be tested; and
b) determining a biomarker signature of the test sample by measuring the presence and/or amount in the test sample of one or more biomarker selected from the group defined in Table A(i) and/or Table A(ii);
wherein the presence and/or amount in the test sample of the one or more biomarker selected from the group defined in Table A(i) and/or Table A(ii) is indicative of the presence of breast cancer cells in the individual.

2. The method according to claim 1 wherein the breast cancer is early breast cancer.

3. The method according to claim 1 or 2 wherein step (b) comprises or consists of measuring the presence and/or amount of 1 or more biomarker listed in Table A, for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113 or 114 of the biomarkers listed in Table A.

4. The method according to any one of the preceding claims wherein step (b) comprises or consists of measuring the presence and/or amount of 1 or more biomarker listed in Table A(i), for example at least 2 or 3 of the biomarkers listed in Table A(i).

5. The method according to any one of the preceding claims wherein step (b) comprises or consists of measuring the presence and/or amount of 1 or more biomarker listed in Table A(ii), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65 of the biomarkers listed in Table A(ii).

6. The method according to any one of the preceding claims wherein step (b) comprises or consists of measuring the presence and/or amount of 1 or more biomarker listed in Table A(iii), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, or 46 of the biomarkers listed in Table A(iii).

7. The method according to any one of the preceding claims wherein step (b) comprises or consists of measuring the presence and/or amount of 1 or more biomarker listed in Table B(i), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68 of the biomarkers listed in Table B(i).

8. The method according to any one of the preceding claims wherein step (b) comprises or consists of measuring the presence and/or amount of 1 or more biomarker listed in Table B(ii), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 of the biomarkers listed in Table B(ii).

9. The method according to claim 8 wherein the method is indicative of whether or not the test sample is characteristic of a sample taken from an individual with breast cancer 0-52 weeks prior to diagnosis by conventional clinical methods.

10. The method according to any one of the preceding claims wherein step (b) comprises or consists of measuring the presence and/or amount of 1 or more biomarker listed in Table B(iii), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 or 43 of the biomarkers listed in Table B(iii).

11. The method according to claim 10 wherein the method is indicative of whether or not the test sample is characteristic of a sample taken from an individual with breast cancer 52-104 weeks prior to diagnosis by conventional clinical methods.

12. The method according to any one of the preceding claims wherein step (b) comprises or consists of measuring the presence and/or amount of 1 or more biomarker listed in Table B(iv), for example at least 2, 3, 4, 5, 6, 7, 8, 9, 10, 11, 12, 13, 14, 15, 16, 17, 18, 19, 20 or 21 of the biomarkers listed in Table B(iv).

13. The method according to claim 12 wherein the method is indicative of whether or not the test sample is characteristic of a sample taken from an individual with breast cancer 0-26 weeks prior to diagnosis by conventional clinical methods.

14. The method according to any one of the preceding claims wherein step (b) comprises or consists of measuring the presence and/or amount of 1 or more biomarker listed in Table B(v), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77 or 78 of the biomarkers listed in Table B(v).

15. The method according to claim 14 wherein the method is indicative of whether or not the test sample is characteristic of a sample taken from an individual with breast cancer 26-52 weeks prior to diagnosis by conventional clinical methods.

16. The method according to any one of the preceding claims wherein step (b) comprises or consists of measuring the presence and/or amount of 1 or more biomarker listed in Table B(vi), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 of the biomarkers listed in Table B(vi).

17. The method according to claim 16 wherein the method is indicative of whether or not the test sample is characteristic of a sample taken from an individual with breast cancer 52-78 weeks prior to diagnosis by conventional clinical methods.

18. The method according to any one of the preceding claims wherein step (b) comprises or consists of measuring the presence and/or amount of 1 or more biomarker listed in Table B(vii), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 of the biomarkers listed in Table B(vii).

19. The method according to claim 18 wherein the method is indicative of whether or not the test sample is characteristic of a sample taken from an individual with breast cancer 78-104 weeks prior to diagnosis by conventional clinical methods.

20. The method according to any one of the preceding claims wherein step (b) comprises or consists of measuring the presence and/or amount of 1 or more biomarker listed in Table B(viii).

21. The method according to claim 20 wherein the method is indicative of whether or not the test sample is characteristic of a sample taken from an individual with breast cancer 0-35 weeks prior to diagnosis by conventional clinical methods of breast cancer consisting of tumours of less than or equal to 20 mm in any dimension.

22. The method according to any one of the preceding claims wherein step (b) comprises or consists of measuring the presence and/or amount of 1 or more biomarker listed in Table B(ix), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37 or 38 of the biomarkers listed in Table B(ix).

23. The method according to claim 22 wherein the method is indicative of whether or not the test sample is characteristic of a sample taken from an individual with breast cancer 70-104 weeks prior to diagnosis by conventional clinical methods of breast cancer consisting of tumours of less than or equal to 20 mm in any dimension.

24. The method according to any one of the preceding claims wherein step (b) comprises or consists of measuring the presence and/or amount of 1 or more biomarker listed in Table B(x), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62 or 63 of the biomarkers listed in Table B(x).

25. The method according to claim 24 wherein the method is indicative of whether or not the test sample is characteristic of a sample taken from an individual with breast cancer 20-26 or 26-52 weeks prior to diagnosis by conventional clinical methods.

26. The method according to any one of the preceding claims wherein step (b) comprises or consists of measuring the presence and/or amount of 1 or more biomarker listed in Table B(xi), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81 or 82 of the biomarkers listed in Table B(xi).

27. The method according to claim 26 wherein the method is indicative of whether or not the test sample is characteristic of a sample taken from an individual with breast cancer 26-52 or 52-78 weeks prior to diagnosis by conventional clinical methods.

28. The method according to any one of the preceding claims wherein step (b) comprises or consists of measuring the presence and/or amount of 1 or more biomarker listed in Table B(xii), for example at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46 or 47 of the biomarkers listed in Table B(xii).

29. The method according to claim 28 wherein the method is indicative of whether or not the test sample is characteristic of a sample taken from an individual with breast cancer 52-78 or 78-104 weeks prior to diagnosis by conventional clinical methods.

30. The method according to any one of the preceding claims wherein step (b) comprises or consists of measuring the presence and/or amount of 1 or more biomarker listed in FIG. 4(C), for example at least 2, 3, 4, 5, 6, 7, 8, 9, 10, 11, 12, 13, 14, 15, 16, 17 or 18 of the biomarkers listed in FIG. 4(C).

31. The method according to claim 30 wherein the method is indicative of whether or not the test sample is characteristic of a sample taken from an individual with breast cancer 0-26 or 26-52 weeks prior to diagnosis by conventional clinical methods.

32. The method according to any one of the preceding claims wherein step (b) comprises or consists of measuring the presence and/or amount of 1 or more biomarker listed in FIG. 4(D), for example at least 2, 3, 4, 5, 6, 7, 8, 9, 10, 11, 12, 13, 14, 15, 16, 17 or 18 of the biomarkers listed in FIG. 4(D).

33. The method according to claim 32 wherein the method is indicative of whether or not the test sample is characteristic of a sample taken from an individual with breast cancer 26-52 or 52-78 weeks prior to diagnosis by conventional clinical methods.

34. The method according to any one of the preceding claims wherein step (b) comprises or consists of measuring the presence and/or amount of 1 or more biomarker listed in FIG. 4(D), for example at least 2, 3, 4, 5, 6, 7, 8, 9, 10, 11, 12, 13, 14, 15, 16, 17 or 18 of the biomarkers listed in FIG. 4(D).

35. The method according to claim 34 wherein the method is indicative of whether or not the test sample is characteristic of a sample taken from an individual with breast cancer 52-78 or 78-104 weeks prior to diagnosis by conventional clinical methods.

36. The method according to any one of the preceding claims wherein step (b) comprises measuring the presence and/or amount of all of the biomarkers listed in Table A.

37. The method according to any one of the preceding claims further comprising or consisting of the steps of:
e) providing one or more control sample from:
i. an individual not afflicted with breast cancer; and/or
ii. an individual afflicted with breast cancer, wherein the sample was taken at a time period defined in claims 7-37 that differs from the time period that the test sample is characteristic of;
f) determining a biomarker signature of the one or more control sample by measuring the presence and/or amount in the control sample of the one or more biomarkers measured in step (b);
wherein the presence of breast cancer is identified in the event that the presence and/or amount in the test sample of the one or more biomarkers measured in step (b) is different from the presence and/or amount in the control sample of the one or more biomarkers measured in step (d).
By “the time period that the test sample is characteristic of” we include the time period prior to breast cancer diagnosis by conventional clinical methods.

38. The method according to any one of the preceding claims further comprising or consisting of the steps of:
g) providing one or more control sample from;
i. an individual afflicted with breast cancer (i.e., a positive control); and/or
ii. an individual afflicted with breast cancer, wherein the sample was taken at a time period defined in claims 7-35 that corresponds to the time period that the test sample is characteristic of;
h) determining a biomarker signature of the control sample by measuring the presence and/or amount in the control sample of the one or more biomarkers measured in step (b);
wherein the presence of breast cancer is identified in the event that the presence and/or amount in the test sample of the one or more biomarkers measured in step (b) corresponds to the presence and/or amount in the control sample of the one or more biomarkers measured in step (f).

39. The method according to claim 37 and/or 38 wherein the control samples comprise one or more sample taken from each of the time periods defined in claims 7-35 to be tested.

40. The method according to claim 37 and/or 38 wherein the control samples comprise one or more sample taken from each of the time periods defined in claims 7-35.

41. The method according to any of claims 37-40, wherein the individual from which the one or more control sample was obtained was not, at the time the sample was obtained, afflicted with breast abscess, breast fibroadenoma, fibroadenoma, fibrocystic breast disease, fibrocystic breasts, gynecomastia, mastalgia and/or mastitis.

42. The method according to any of claims 37-41, wherein the individual from which the one or more control sample was obtained was not, at the time the sample was obtained, afflicted with any disease or condition of the breast.

43. The method according to claim 37, wherein the individual not afflicted with breast cancer was not, at the time the sample was obtained, afflicted with any disease or condition.

44. The method according to claim 37 wherein the individual not afflicted with breast cancer is a healthy individual.

45. The method according to any one of claims 37-41 wherein the one or more individual afflicted with breast cancer is afflicted with a breast cancer selected from the group consisting of ductal carcinoma in situ (DCIS), lobular carcinoma in situ (LCIS), invasive ductal breast cancer, invasive lobular breast cancer, Inflammatory breast cancer, medullary breast cancer, mucinous (mucoid or colloid) breast cancer, tubular breast cancer, adenoid cystic carcinoma of the breast (cribriform breast cancer), metaplastic breast cancer, angiosarcoma of the breast, lymphoma of the breast, basal type breast cancer, malignant phyllodes or cystosarcoma phyllodes and papillary breast cancer.

46. The method according to any one of the preceding claims wherein the breast cancer is invasive ductal breast cancer.

47. The method according to any one of the preceding claims wherein the method is repeated.

48. The method according to any one of the preceding claims wherein the method is repeated and wherein, in step (a), the sample to be tested is taken at different time to the previous method repetition.

49. The method according to claim 47 or 48 wherein the method is repeated using a test sample taken at a different time period to the previous test sample(s) used.

50. The method according to claim 48 or 49 wherein the method is repeated using a test sample taken between 1 day to 104 weeks to the previous test sample(s) used, for example, between 1 week to 100 weeks, 1 week to 90 weeks, 1 week to 80 weeks, 1 week to 70 weeks, 1 week to 60 weeks, 1 week to 50 weeks, 1 week to 40 weeks, 1 week to 30 weeks, 1 week to 20 weeks, 1 week to 10 weeks, 1 week to 9 weeks, 1 week to 8 weeks, 1 week to 7 weeks, 1 week to 6 weeks, 1 week to 5 weeks, 1 week to 4 weeks, 1 week to 3 weeks, or 1 week to 2 weeks.

51. The method according to claim 48 or 49 wherein the method is repeated using a test sample taken every period from the group consisting of: 1 day, 2 days, 3 day, 4 days, 5 days, 6 days, 7 days, 10 days, 2 weeks, 3 weeks, 4 weeks, 5 weeks, 6 weeks, 7 weeks, 8 weeks, 9 weeks, 10 weeks, 15 weeks, 20 weeks, 25 weeks, 30 weeks, 35 weeks, 40 weeks, 45 weeks, 50 weeks, 55 weeks, 60 weeks, 65 weeks, 70 weeks, 75 weeks, 80 weeks, 85 weeks, 90 weeks, 95 weeks, 100 weeks, 104, weeks, 105 weeks, 110 weeks, 115 weeks, 120 weeks, 125 weeks and 130 weeks.

52. The method according to any one of claims 47-51 wherein the method is repeated at least once, for example, 2 times, 3 times, 4 times, 5 times, 6 times, 7 times, 8 times, 9 times, 10 times, 11 times, 12 times, 13 times, 14 times, 15 times, 16 times, 17 times, 18 times, 19 times, 20 times, 21 times, 22 times, 23, 24 times or 25 times.

53. The method according to any one of claims 47-51 wherein the method is repeated continuously.

54. The method according to any one of claims 47-51 wherein the method is repeated until diagnosis of breast cancer in the individual using conventional clinical methods.

55. The method according to any one of claims 47-54 wherein each repetition uses test sample taken from the same individual.

56. The method according to any one of claims 1 to 55 wherein step (b) comprises measuring the expression of the protein or polypeptide of the one or more biomarker(s)

57. The method according to any one of the preceding claims wherein step (b), (d) and/or step (f) is performed using one or more first binding agent capable of binding to a biomarker listed in Table A or Table B.

58. The method according to claim 57 wherein the first binding agent comprises or consists of an antibody or an antigen-binding fragment thereof.

59. The method according to claim 58 wherein the antibody or antigen-binding fragment thereof is a recombinant antibody or antigen-binding fragment thereof.

60. The method according to claim 58 or 59 wherein the antibody or antigen-binding fragment thereof is selected from the group consisting of: scFv; Fab; a binding domain of an immunoglobulin molecule.

61. The method according to any one of claims 58 to 60 wherein the first binding agent is immobilised on a surface.

62. The method according to any one of claims 57 to 61 wherein the one or more biomarkers in the test sample are labelled with a detectable moiety.

63. The method according to any one of claims 57 to 62 wherein the one or more biomarkers in the control sample(s) are labelled with a detectable moiety.

64. The method according to claim 62 or 63 wherein the detectable moiety is selected from the group consisting of: a fluorescent moiety; a luminescent moiety; a chemiluminescent moiety; a radioactive moiety; an enzymatic moiety.

65. The method according to claim 62 or 63 wherein the detectable moiety is biotin.

66. The method according to any one of claims 57 to 63 wherein step (b), (d) and/or step (f) is performed using an assay comprising a second binding agent capable of binding to the one or more biomarkers, the second binding agent comprising a detectable moiety.

67. The method according to any one of claim 66 wherein the second binding agent comprises or consists of an antibody or an antigen-binding fragment thereof.

68. The method according to claim 67 wherein the antibody or antigen-binding fragment thereof is a recombinant antibody or antigen-binding fragment thereof.

69. The method according to claim 67 or 68 wherein the antibody or antigen-binding fragment thereof is selected from the group consisting of: scFv; Fab; a binding domain of an immunoglobulin molecule.

70. The method according to any one of claims 68 to 71 wherein the detectable moiety is selected from the group consisting of: a fluorescent moiety; a luminescent moiety; a chemiluminescent moiety; a radioactive moiety; an enzymatic moiety.

71. The method according to claim 69 wherein the detectable moiety is fluorescent moiety (for example an Alexa Fluor dye, e.g. Alexa647).

72. The method according to any one of the preceding claims wherein the method comprises or consists of an ELISA (Enzyme Linked Immunosorbent Assay).

73. The method according to any one of the preceding claims wherein step (b), (d) and/or step (f) is performed using an array.

74. The method according to claim 73 wherein the array is a bead-based array.

75. The method according to claim 73 wherein the array is a surface-based array.

76. The method according to any one of claims 73 to 75 wherein the array is selected from the group consisting of: macroarray; microarray; nanoarray.

77. The method according to any one of the preceding claims wherein the method comprises:
(v) labelling biomarkers present in the sample with biotin;
(vi) contacting the biotin-labelled proteins with an array comprising a plurality of scFv immobilised at discrete locations on its surface, the scFv having specificity for one or more of the proteins in Table A or B;
(vii) contacting the immobilised scFv with a streptavidin conjugate comprising a fluorescent dye; and
(viii) detecting the presence of the dye at discrete locations on the array surface
wherein the expression of the dye on the array surface is indicative of the expression of a biomarker from Table III in the sample.

78. The method according to any one of claims 1 to 55 wherein, step (b), (d) and/or (f) comprises measuring the expression of a nucleic acid molecule encoding the one or more biomarkers.

79. The method according to claim 78, wherein the nucleic acid molecule is a cDNA molecule or an mRNA molecule.

80. The method according to claim 78, wherein the nucleic acid molecule is an mRNA molecule.

81. The method according to claim 78, 79 or 80, wherein measuring the expression of the one or more biomarker(s) in step (b), (d) and/or (f) is performed using a method selected from the group consisting of Southern hybridisation, Northern hybridisation, polymerase chain reaction (PCR), reverse transcriptase PCR (RT-PCR), quantitative real-time PCR (qRT-PCR), nanoarray, microarray, macroarray, autoradiography and in situ hybridisation.

82. The method according to any one of claims 78-81, wherein measuring the expression of the one or more biomarker(s) in step (b) is determined using a DNA microarray.

83. The method according to any one of claims 78-82, wherein measuring the expression of the one or more biomarker(s) in step (b), (d) and/or (f) is performed using one or more binding moieties, each individually capable of binding selectively to a nucleic acid molecule encoding one of the biomarkers identified in Table A or Table B.

84. The method according to claim 83, wherein the one or more binding moieties each comprise or consist of a nucleic acid molecule.

85. The method according to claim 84 wherein, the one or more binding moieties each comprise or consist of DNA, RNA, PNA, LNA, GNA, TNA or PMO.

86. The method according to claim 84 or 85, wherein the one or more binding moieties each comprise or consist of DNA.

87. The method according to any one of claims 84-86 wherein the one or more binding moieties are 5 to 100 nucleotides in length.

88. The method according to any one of claims 84-86 wherein the one or more nucleic acid molecules are 15 to 35 nucleotides in length.

89. The method according to any one of claims 84-88 wherein the binding moiety comprises a detectable moiety.

90. The method according to claim 89 wherein the detectable moiety is selected from the group consisting of: a fluorescent moiety; a luminescent moiety; a chemiluminescent moiety; a radioactive moiety (for example, a radioactive atom); or an enzymatic moiety.

91. The method according to claim 90 wherein the detectable moiety comprises or consists of a radioactive atom.

92. The method according to claim 91 wherein the radioactive atom is selected from the group consisting of technetium-99m, iodine-123, iodine-125, iodine-131, indium-111, fluorine-19, carbon-13, nitrogen-15, oxygen-17, phosphorus-32, sulphur-35, deuterium, tritium, rhenium-186, rhenium-188 and yttrium-90.

93. The method according to claim 90 wherein the detectable moiety of the binding moiety is a fluorescent moiety.

94. The method according to any one of the preceding claims wherein, the sample provided in step (a), (c) and/or (e) is selected from the group consisting of unfractionated blood, plasma, serum, tissue fluid, breast tissue, milk, bile and urine.

95. The method according to claim 94, wherein the sample provided in step (a), (c) and/or (e) is selected from the group consisting of unfractionated blood, plasma and serum.

96. The method according to claim 94 or 95, wherein the sample provided in step (a), (c) and/or (e) is serum.

97. The method according to any one of the preceding claims wherein the predicative accuracy of the method, as determined by an ROC AUC value, is at least 0.50, for example at least 0.55, 0.60, 0.65, 0.70, 0.75, 0.80, 0.85, 0.90, 0.95, 0.96, 0.97, 0.98 or at least 0.99.

98. The method according to claim 97 wherein the predicative accuracy of the method, as determined by an ROC AUC value, is at least 0.70.

99. The method according to any one of the preceding claims wherein, in the event that the individual is diagnosed with breast cancer, the method comprises the step of:
(g) providing the individual with breast cancer therapy.

100. The method according to claim 99 wherein the breast cancer therapy is selected from the group consisting of surgery, chemotherapy, immunotherapy, chemoimmunotherapy and thermochemotherapy.

101. An array for determining the presence of breast cancer in an individual comprising one or more binding agent as defined in any one of claims 57-72 and 79-93.

102. The array according to claim 101 wherein the one or more binding agents is capable of binding to all of the proteins defined in Table A or Table B.

103. Use of one or more biomarkers selected from the group defined in Table A or Table B as a biomarker for determining the presence of breast cancer in an individual.

104. The use according to claim 103 wherein all of the proteins defined in Table A or Table B are used as a diagnostic marker for determining the presence of breast cancer in an individual.

105. The use of one or more binding moiety as defined in any one of claims 57-72 and 79-93 for determining the presence of breast cancer in an individual.

106. The use according to claim 105 wherein biomarkers for all of the proteins defined in Table A or Table B are used.

107. A kit for determining the presence of breast cancer comprising:
C) one or more binding agent as defined in any one of claims 57-72 and 79-93 or an array according to claims 73-77 or claim 101-102;
D) instructions for performing the method as defined in any one of claims 1-100.

108. A method of treating breast cancer in an individual comprising the steps of:
(a) diagnosing breast cancer according to the method defined in any one of claims 1-100; and
(b) providing the individual with breast cancer therapy.

109. The method according to claim 108 wherein the breast cancer therapy is selected from the group consisting of surgery, chemotherapy, immunotherapy, chemoimmunotherapy and thermochemotherapy.

110. A method or use for determining the presence of breast cancer in an individual substantially as described herein.

111. An array or kit for determining the presence of breast cancer in an individual substantially as described herein.


SEQUENCE LISTING (20 sequences)

SEQ ID NO: 1 - protein, 242 aa, Homo sapiens
MAEVQLLESGGGLVQPGGSLRLSCAASGFTFSSYGFHWVRQAPGKGLEWVSLISWDGGSTYYADSVKGRFTISRDNSKNTLYLQMNSLRAEDTAVYYCARGTWFDPWGQGTLVTVSSGGGGSGGGGSGGGGSQSVLTQPPSASGTPGQRVTISCSGSSSNIGNNAVNWYQQLPGTAPKLLIYRNNQRPSGVPDRFSGSKSGTSASLAISGLRSEDEADYYCAAWDDSLSWVFGGGTKLTVLG

SEQ ID NO: 2 - protein, 31 aa, Artificial Sequence
  6 x histidine affinity tag
DYKDHDGDYKDHDIDYKDDDDKAAAHHHHHH

SEQ ID NO: 3 - protein, 10 aa, Artificial Sequence
  CIMS
SGSGTEEQLK

SEQ ID NO: 4 - protein, 10 aa, Artificial Sequence
  CIMS
SGSGLSADHR

SEQ ID NO: 5 - protein, 10 aa, Artificial Sequence
  CIMS
SGSGDFAEDK

SEQ ID NO: 6 - protein, 18 aa, Artificial Sequence
  CIMS
SGSGFLLMQYGGMDEHAR

SEQ ID NO: 7 - protein, 15 aa, Artificial Sequence
  CIMS
SGSGGIVKYLYEDEG

SEQ ID NO: 8 - protein, 8 aa, Artificial Sequence
  CIMS
SGSGEDFR

SEQ ID NO: 9 - protein, 8 aa, Artificial Sequence
  CIMS
SGSGEPFR

SEQ ID NO: 10 - protein, 10 aa, Artificial Sequence
  CIMS
SGSGLNVWGK

SEQ ID NO: 11 - protein, 10 aa, Artificial Sequence
  CIMS
SGSGLTEFAK

SEQ ID NO: 12 - protein, 18 aa, Artificial Sequence
  CIMS
SGSGLWETVQKWREYRRQ

SEQ ID NO: 13 - protein, 10 aa, Artificial Sequence
  CIMS
SGSGLYEIAR

SEQ ID NO: 14 - protein, 10 aa, Artificial Sequence
  CIMS
SGSGQEASFK

SEQ ID NO: 15 - protein, 10 aa, Artificial Sequence
  CIMS
SGSGSEAHLR

SEQ ID NO: 16 - protein, 10 aa, Artificial Sequence
  CIMS
SGSGSSAYSR

SEQ ID NO: 17 - protein, 10 aa, Artificial Sequence
  CIMS
SGSGSYVSLK

SEQ ID NO: 18 - protein, 10 aa, Artificial Sequence
  CIMS
SGSGTLYVGK

SEQ ID NO: 19 - protein, 19 aa, Artificial Sequence
  CIMS
SGSGWTRNSNMNYWLIIRL

SEQ ID NO: 20 - protein, 8 aa, Artificial Sequence
  CIMS
SGSGWDSR